(19) United States
(12) Patent Application Publication
(10) Pub. No.: US 2022/0389310 A1
(43) Pub. Date: Dec. 8, 2022

(54) LUMINESCENT MATERIALS AND METHODS THEREOF

(71) Applicant: University of Washington, Seattle, WA (US)

(72) Inventors: Yunping Huang, Seattle, WA (US); Christine Keiko Luscombe, III, Seattle, WA (US)

(73) Assignee: University of Washington, Seattle, WA (US)

(21) Appl. No.: 17/762,599

(22) PCT Filed: Sep. 24, 2020
(86) PCT No.: PCT/US2020/052553

§ 371 Date: Mar. 22, 2022

Related U.S. Application Data
(60) Provisional application No. 62/907,050, filed on Sep. 27, 2019.

(51) Int. Cl. C09K 11/06; C07D 519/00; C07D 473/12; H01L 51/00

(52) U.S. Cl. CPC C09K 11/06; C07D 519/00; C07D 473/12; H01L 51/0054; H01L 51/0072; C09K 2211/1018; C09K 2211/1007; C09K 2211/1014; C09K 2211/1011; H01L 2251/552; H01L 51/5012

(57) ABSTRACT

The present disclosure features a luminescent molecule, including a luminophore (e.g., a fluorescent dye); and a moiety including a heteroaryl core covalently and directly bonded to the luminophore. The luminescent molecule has an increased photoluminescence quantum yield relative to an analogous luminophore without a covalently bonded moiety including the heteroaryl core.

### CROSS-REFERENCE(S) TO RELATED APPLICATION(S)

This application claims the benefit of U.S. Patent Application No. 62/907,050, filed Sep. 27, 2019, the disclosure of which is incorporated herein by reference in its entirety.

### STATEMENT OF GOVERNMENT LICENSE RIGHTS

This invention was made with government support under Grant No. CHE-1700982, awarded by the National Science Foundation. The government has certain rights in the invention.

### BACKGROUND

Cost, performance, and stability are three key aspects for assessing commercial viability of new technologies. In the past decades, the emergence of organic semiconductors has significantly altered previous understanding of electronic devices. Organic semiconductors are π-conjugated molecules that are lightweight and pliable, and are the driving force behind flexible electronics. By attaching side chains to these π-conjugated molecules, organic semiconductors can be provided with solution processability, which can enable low-cost and efficient device manufacturing, such as inkjet printing and roll-to-roll printing. This can reduce the time and capital commitments in device fabrication. Solution-processed devices, such as organic light emitting diodes (OLEDs), organic solar cells (OSCs), and organic field-effect transistors (OFETs), can achieve improved efficiency due to materials design and device fabrication advances.

However, aggregation caused quenching (ACQ) of fluorescence is commonly observed in organic luminophores. While there are a number of reasons why ACQ can take place, for organic luminophores with extended π-conjugation, such as pyrene and perylene diimide, the organic luminophores can interact with each other via the 11 orbital overlap to form non-fluorescent excimers. Aggregation causes non-radiative thermal decay, which dissipates energy in the form of heat rather than light emission, further resulting in low photoluminescence quantum yields (PLQYs). Thus, ACQ limits the efficiency of organic luminophores in luminescence applications, such as organic light emitting diodes (OLEDs), hybrid light emitting diodes (hybrid LEDs), organic laser, luminescent solar concentrators (LSCs), bio-sensing, bio-imaging, and down-converting devices.

To date, numerous efforts have been devoted to suppressing ACQ in organic luminophores including blending, co-crystallization, and covalently attaching functional moieties. Existing methods for suppressing ACQ can provide luminescent materials that are suitable for fluorescent devices, but carry environmental concerns. In particular, existing methods heavily rely on Suzuki coupling, which uses air-sensitive and explosive organolithium reagents to prepare boronic ester precursors. If luminescent materials are to be prepared at a commercial scale, a method that avoids organolithium reagents would substantially diminish workplace safety risks. Moreover, organolithium reagents decrease functional group tolerance in the synthesis, because they are strongly nucleophilic.

Furthermore, current organic semiconductors are generally unstable, thus limiting the lifespans of commercial technologies such as OLEDs and preventing the wide-ranged commercial adoptions of new technologies. The instability of organic semiconductors is strongly associated with the reactivity towards ground-state oxygen (³O₂) in the atmosphere. In addition, damaging radicals are generated with the presence of side chains that are incorporated in organic semiconductors to increase their processability. Side chains thus present advantages and disadvantages for organic semiconductors: they enables low-cost device fabrication via solution processing but also reduce stability of the organic semiconductors.

There is a need for stable luminescent materials that have little ACQ and that can be synthesized using environmentally-friendly and scalable methods. The present application fulfills these needs and provides further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a luminescent molecule, including:

a luminophore; and

a moiety including a purine core covalently and directly bonded to the luminophore, the purine core optionally substituted with 1, 2, 3, or 4 substituents independently selected from C═O, amino, C₁₋₂₄ alkyl, C₂₋₂₄ alkenyl, C₂₋₂₄ alkynyl, poly(alkylene oxide), aryl, heteroaryl, a polysiloxane, and any combination thereof, wherein each substituent is optionally substituted with 1, 2, 3, or 4 substituents independently selected from C₁₋₂₄ alkyl, C₂₋₂₄ alkenyl, C₂₋₂₄ alkynyl, halo, SH, cyclic ether (e.g., epoxide), OH, CN, N₃, NCO, C(O)H, COOH, C(O)OR^{a}, C(O)NR^{a}, N═NR^{a}, aryl, heteroaryl, SO₃⁻, C—PO(OR^{a})(OR^{b}), NH₂, NHR^{a}, NR^{a}R^{b}, and (NR^{a}R^{b}R^{c})⁺, wherein each of R^{a}, R^{b}, and R^{c} is independently H, alkyl, aryl, arylalkyl, or a heterocycle,

wherein the luminescent molecule has an increased photoluminescence quantum yield relative to an analogous luminophore dye without a covalently bonded moiety including the purine core.

In another aspect, the present disclosure features a method of making a luminescent molecule, including:

providing a reaction mixture including a luminophore including (i.e., having) a leaving group covalently bound thereto, a compound including a purine core and an activatable C—H bond, and a palladium or copper catalyst;

activating the activatable C—H bond of the compound including the purine core with the palladium or copper catalyst to provide an activated compound including the purine core, and

reacting the luminophore including a leaving group covalently bound thereto and the activated compound including the purine core to provide a luminescent molecule described herein.

In yet another aspect, the present disclosure features a device including a luminescent molecule described herein.

In yet a further aspect, the present disclosure features a fluorescent biomolecule label, including a luminescent molecule described herein.

### DESCRIPTION OF THE DRAWINGS

The foregoing aspects and many of the attendant advantages of the present disclosure will become more readily appreciated as the same become better understood by reference to the following detailed description, when taken in conjunction with the accompanying drawings.

FIG. 1 is a schematic representation of embodiments of luminophores for making the luminescent molecules of the present disclosure.

FIG. 2 is a schematic representation of embodiments of the electron density comparison between theobromine and representative luminophores.

FIG. 3A is a schematic representation of an embodiment of a synthesis method of embodiments of luminescent molecules of the present disclosure.

FIG. 3B is a schematic representation of embodiments of synthesis methods of embodiments of luminescent molecules of the present disclosure.

FIG. 3C is a schematic representation of embodiments of synthesis methods of embodiments of luminescent molecules of the present disclosure.

FIG. 3D is a schematic representation of an embodiment of a method of synthesis of embodiments of luminescent molecules of the present disclosure.

FIG. 4A is a plot of the absorption spectra and photoluminescence spectra of an embodiment of a luminescent molecule of the present disclosure (PT1), in chloroform at a concentration of 10⁻⁵M, and as a thin film, at room temperature.

FIG. 4B is a plot of the absorption spectra and photoluminescence spectra of an embodiment of a luminescent molecule of the present disclosure (PT2), in chloroform at a concentration of 10⁻⁵M, and as a thin film, at room temperature.

FIG. 4C is a plot of the absorption spectra and photoluminescence spectra of an embodiment of a luminescent molecule of the present disclosure (PT4), in chloroform at a concentration of 10⁻⁵M, and as a thin film, at room temperature.

FIG. 5 is a cyclic voltammogram of embodiments of luminescent molecules of the present disclosure, with ferrocene as a reference; and the accompanying highest occupied molecular orbitals (HOMOs) and lowest unoccupied molecular orbitals (LUMOs) of the luminescent molecules.

FIG. 6A is a plot of a transient absorption of an embodiment of luminescent molecule of the present disclosure (PT1), as a thin film on glass substrate. Thin film samples were prepared by spin-coating 10 mL chloroform solutions of the embodiment of luminescent molecule onto glass substrates in rate of 1000 rpm.

FIG. 6B is a plot of a transient absorption of an embodiment of luminescent molecule of the present disclosure (PT2), as a thin film on glass substrate. Thin film samples were prepared by spin-coating 10 mL chloroform solutions of the embodiment of luminescent molecule onto glass substrates in rate of 1000 rpm.

FIG. 6C is a plot of a transient absorption of an embodiment of luminescent molecule of the present disclosure (PT4), as a thin film on glass substrate. Thin film samples were prepared by spin-coating 10 mL chloroform solutions of the embodiment of luminescent molecule onto glass substrates in rate of 1000 rpm.

FIG. 7A is a plot of a photoluminescence lifetime of an embodiment of luminescent molecule of the present disclosure (PT1).

FIG. 7B is a plot of a photoluminescence lifetime of an embodiment of luminescent molecule of the present disclosure (PT2).

FIG. 7C is a plot of a photoluminescence lifetime of an embodiment of luminescent molecule of the present disclosure (PT4).

FIG. 8A is a graphical representation of a crystal structure of an embodiment of luminescent molecule of the present disclosure (PT1). H atoms are not shown for clarity.

FIG. 8B is a graphical representation of a crystal structure of an embodiment of luminescent molecule of the present disclosure (PT2). H atoms are not shown for clarity.

FIG. 8C is a graphical representation of a crystal structure of an embodiment of luminescent molecule of the present disclosure (PT4). H atoms are not shown for clarity.

FIG. 9 is an illustration of a density function theory model of HOMOs and LUMOs of embodiments of luminescent molecules of the present disclosure.

FIG. 10A is a plot of the photoluminescence spectra of an embodiment of a luminescent molecule of the present disclosure (PT1) in a variety of solvents, at a concentration of 10⁻⁵M at room temperature.

FIG. 10B is a plot of the photoluminescence spectra of an embodiment of a luminescent molecule of the present disclosure (PT2) in a variety of solvents, at a concentration of 10⁻⁵M at room temperature.

FIG. 10C is a plot of the photoluminescence spectra of an embodiment of a luminescent molecule of the present disclosure (PT4) in a variety of solvents, at a concentration of 10⁻⁵M at room temperature.

FIG. 11A is a plot of the absorption spectra of an embodiment of a luminescent molecule of the present disclosure (PT1) in a variety of solvents, at a concentration of 10⁻⁵M at room temperature.

FIG. 11B is a plot of the absorption spectra of an embodiment of a luminescent molecule of the present disclosure (PT2) in a variety of solvents, at a concentration of 10⁻⁵M at room temperature.

FIG. 11C is a plot of the absorption spectra of an embodiment of a luminescent molecule of the present disclosure (PT4) in a variety of solvents, at a concentration of 10⁻⁵M at room temperature.

FIG. 12A is a plot of the photoluminescence spectra of an embodiment of a luminescent molecule of the present disclosure (PT1) at different temperatures, at a concentration of 10⁻⁵M and at an excitation wavelength of 322 nm.

FIG. 12B is a plot of the photoluminescence spectra of an embodiment of a luminescent molecule of the present disclosure (PT2) at different temperatures, at a concentration of 10⁻⁵M and at an excitation wavelength of 322 nm.

FIG. 12C is a plot of the photoluminescence spectra of an embodiment of a luminescent molecule of the present disclosure (PT4) at different temperatures, at a concentration of 10⁻⁵M and at an excitation wavelength of 322 nm.

FIG. 13 is an energy level plot of 4-dimethylaminobenzonitrile as a function of the dihedral angle between the dimethylamino group and benzene ring.

FIG. 14A is a plot of the emission spectra of a film of an embodiment of a luminescent molecule of the present disclosure (PT1), collected from an edge of the film with increasing excitation fluence. A plot of output intensity from the film edge as a function of the excitation fluence is shown under the emission spectra.

FIG. 14B is a plot of the emission spectra of a film of an embodiment of a luminescent molecule of the present disclosure (PT2), collected from an edge of the film with increasing excitation fluence. A plot of output intensity from the film edge as a function of the excitation fluence is shown under the emission spectra.

FIG. 14C is a plot of the emission spectra of a film of an embodiment of a luminescent molecule of the present disclosure (PT4), collected from an edge of the film with increasing excitation fluence. A plot of output intensity from the film edge as a function of the excitation fluence is shown under the emission spectra.

FIG. 15 is a schematic representation of an experimental setup for a stability measurement of embodiments of luminescent molecules of the present disclosure.

FIG. 16 is a schematic representation of an embodiment of a synthesis method of embodiments of luminescent molecules (BT2 and NT2) of the present disclosure. The reaction conditions are a) K₂CO₃, DMF at 120° C. overnight; b) Pd₂(dba)₃, P(o-methoxyphenyl)₃, Cs₂CO₃, pivalic acid, xylene at 100° C. overnight.

FIG. 17A is a plot of the absorption (ABS) and photoluminescence (PL) spectra of an embodiment of a luminescent molecule (BT2) of the present disclosure in 10⁻⁵ M chloroform solution and as thin films.

FIG. 17B is a plot of the absorption (ABS) and photoluminescence (PL) spectra of an embodiment of a luminescent molecule (NT2) of the present disclosure in 10⁻⁵ M chloroform solution and as thin films.

FIG. 18 is a series of photographs comparing the light converting efficacy of inorganic phosphors and luminophores/luminescent molecules. Dye:SBS weight ratio are shown in the photographs.

FIG. 19 is a schematic representation of structures of embodiments of luminophores/luminescent molecules and poly (styrene-butadiene-styrene) (SBS) used in the fabrication of light-converting films.

FIG. 20A is a plot of UPS results of embodiments of luminophores or embodiments of luminescent molecules of the present disclosure.

FIG. 20B is a plot of UPS results of embodiments of luminophores or embodiments of luminescent molecules of the present disclosure.

FIG. 20C is a plot of UPS results of embodiments of luminophores or embodiments of luminescent molecules of the present disclosure.

FIG. 21 is a plot of thin films absorption spectra of embodiments of luminophores or embodiments of luminescent molecules of the present disclosure.

FIG. 22 is a graph showing frontal band structures of luminophores or embodiments of luminescent molecules of the present disclosure.

FIG. 23 is a plot of the photoluminescence decays of embodiments of luminophores under 450 nm radiation in air. The photoluminescence of DCJTB was measured ex situ with an integrating sphere because of ACQ and thus low fluorescence as thin film.

FIG. 24A is a plot of comparing the stabilities of an embodiments of a luminophore as a thin film and a SBS complex.

FIG. 24B is a plot of comparing the stabilities of an embodiments of a luminophore as a thin film and a SBS complex.

FIG. 24C is a plot of comparing the stabilities of an embodiments of a luminescent molecule as a thin film and a SBS complex.

FIG. 24D is a plot of comparing the stabilities of an embodiments of a luminophore as a thin film and a SBS complex.

FIG. 24E is a plot of comparing the stabilities of an embodiments of a luminescent molecule as a thin film and a SBS complex.

FIG. 24F is a plot of comparing the stabilities of an embodiments of a luminescent molecule as a thin film and a SBS complex.

FIG. 25A is plot showing singlet oxygen generation from an embodiment of a luminescent molecule.

FIG. 25B is plot showing singlet oxygen generation from an embodiment of a luminophore.

FIG. 25C is plot showing singlet oxygen generation from an embodiment of a luminescent molecule.

FIG. 25D is plot showing singlet oxygen generation from an embodiment of a luminescent molecule.

FIG. 25E is plot showing singlet oxygen generation from an embodiment of a luminescent molecule.

FIG. 25F is plot showing singlet oxygen generation from an embodiment of a luminescent molecule.

### DETAILED DESCRIPTION

The present disclosure describes luminescent molecules and methods of synthesizing and using the luminescent molecules. The synthesis methods include direct arylation of a luminophore with a moiety including a heterocyclic core, which offers a green and atom-efficient method to form C—C bonds between aromatic building blocks. The direct formation of C—C bond avoids the use of dangerous and costly organolithium reagents, which is necessary for synthesis of precursors (e.g., organotin, boronic acid, and/or boronic ester precursors), in a conventional Suzuki coupling reaction. Therefore, the risks of fire and explosion are reduced in the synthesis methods of the present disclosure, and cost and productivity are optimized. Moreover, direct arylation is compatible with large or commercial-scale production; and flow chemistry can be successfully implemented to further enhance productivity. The synthesis methods do not include highly reactive intermediates, such as organolithium and Grignard reagents, and the reactants can have improved compatibility with one another, compared to reactions involving organolithiums and Grignard reagents.

The luminescent molecules of the present application are thus made by direct arylation in environmentally-friendly (e.g., green) methods to provide luminescent molecules having suppressed ACQ. The materials can be solution-processed and can be scaled up for use in low-cost device fabrication.

In some embodiments, by covalently attaching functionalized purine moieties, such as xanthine derivatives, to organic chromophores via direct arylation, luminescent molecules (e.g., organic semiconductors, “OSs”) that are highly emissive in solid state can be synthesized. The luminescent molecules can possess photoluminescence quantum yields (PLQYs) approaching 100% both in solution and in solid form. In some embodiments, the luminescent materials are made in 2 steps from commercially available starting materials.

In stark contrast to the use of organolithium in a reaction mixture, the key reactants (e.g., xanthine derivatives) in the methods of the present disclosure, are inexpensive, non-hazardous, and readily available natural products, which simultaneously lowers safety concerns and cost in manufacture. Direct arylation allows direct cross-coupling between aromatic moieties, bypassing extra steps required for precursors synthesis, which further enhances manufacture efficiency. Plus, aromatic derivatives (e.g., xanthine derivatives) can be easily functionalized via N-substitution. Thus, the processability and functionalities of final products can be tuned with different functional groups, optimizing their performance and expanding their applications. In addition, the final luminescent molecules have enhanced photostability and processability, compared to luminophores that have not been arylated with a moiety including a heterocyclic core.

### Definitions

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds of the disclosure are stable. As used herein “stable” refers to a compound that is sufficiently robust to survive isolation to a useful degree of purity from a reaction mixture.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl. The term “substituted” in reference to alkyl, alkylene, aryl, arylalkyl, alkoxy, heterocyclyl, heteroaryl, carbocyclyl, siloxanyl, etc., for example, “substituted alkyl”, “substituted alkylene”, “substituted aryl”, “substituted arylalkyl”, “substituted heterocyclyl”, “substituted carbocyclyl”, and “substituted siloxanyl” means alkyl, alkylene, aryl, arylalkyl, heterocyclyl, carbocyclyl, siloxanyl, respectively, in which one or more hydrogen atoms are each independently replaced with a non-hydrogen substituent. Typical substituents include, but are not limited to, alkyl, alkenyl, alkynyl, —X, —R′, —O⁻, ═O, —OR′, —SR′, —S⁻, —NR′₂, —N⁺R′₃, ═NR′, —CX₃, —CN, —OCN, —SCN, —N═C═O, —NCS, —NO, —NO₂, ═N₂, —N₃, —NHC(═O)R′, —OC(═O)R′, —NHC(═O)NR′₂, —S(═O)₂—, —S(═O)₂OH, —S(═O)₂R′, —OS(═O)₂OR′, —S(═O)₂NR′₂, —S(═O)R′, —OP(═O)(OR′)₂, —P(═O)(OR′)₂, —P(═O)(O⁻)₂, —P(═O)(OH)₂, —P(O)(OR′)(O⁻), —C(═O)R′, —C(═O)X, —C(S)R′, —C(O)OR′, —C(O)O⁻, —C(S)OR′, —C(O)SR′, —C(S)SR′, —C(O)NR′₂, —C(S)NR′₂, —C(═NR′)NR′₂, where each X is independently a halogen: F, Cl, Br, or I; and each R′ is independently H, alkyl, aryl, arylalkyl, a heterocycle, or a protecting group. Alkylene, alkenylene, and alkynylene groups may also be similarly substituted. Unless otherwise indicated, when the term “substituted” is used in conjunction with groups such as arylalkyl, which have two or more moieties capable of substitution, the substituents can be attached to the aryl moiety, the alkyl moiety, or both.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(O)C₁-C₆ alkyl indicate the same functionality; similarly arylalkyl and -alkylaryl indicate the same functionality.

As used herein, the term “alkyl” refers to a straight or branched chain saturated hydrocarbon containing from 1 to 24 carbon atoms, unless otherwise specified.

Representative examples of alkyl include, but are not limited to, methyl (Me), ethyl (Et), propyl (e.g., n-propyl, iso-propyl), butyl (e.g., n-butyl, sec-butyl, iso-butyl, tert-butyl), pentyl (n-pentyl, isopentyl, neopentyl), n-hexyl, 3-methylhexyl, 2,2-dimethylpentyl, 2,3-dimethylpentyl, n-heptyl, n-octyl, n-nonyl, and n-decyl. In some embodiments, alkyl contains 1 to 20 carbon atoms, 1 to 16 carbon atoms, 1 to 12 carbon atoms, 1 to 8 carbon atoms, or 1 to 6 carbon atoms.

As used herein, the term “alkylene” refers to a linking alkyl group, i.e., a saturated, branched or straight chain or cyclic hydrocarbon radical having two monovalent radical centers derived by the removal of two hydrogen atoms from the same or two different carbon atoms of a parent alkane. For example, an alkylene group can have 1 to 20 carbon atoms, 1 to 16 carbon atoms, 1 to 12 carbon atoms, 1 to 10 carbon atoms, 1 to 8 carbon atoms, or 1 to 6 carbon atoms. Typical alkylene radicals include, but are not limited to, methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), 1,4-butyl (—CH₂CH₂CH₂CH₂—), and the like.

As used herein, the term “alkenyl” refers to a straight or branched chain hydrocarbon containing from 2 to 24 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl. In some embodiments, alkenyl contains 2 to 20 carbon atoms, 2 to 16 carbon atoms, 2 to 12 carbon atoms, 2 to 8 carbon atoms, or 2 to 6 carbon atoms.

As used herein, the term “alkenylene” refers to a linking alkenyl group, i.e., to an unsaturated, branched or straight chain or cyclic hydrocarbon radical having two monovalent radical centers derived by the removal of two hydrogen atoms from the same or two different carbon atoms of a parent alkene. For example, and alkenylene group can have 2 to 20 carbon atoms, 2 to 16 carbon atoms, 2 to 12 carbon atoms, 2 to 8 carbon atoms, or 2 to 6 carbon atoms. Typical alkenylene radicals include, but are not limited to, 1,2-ethylene (—CH═CH—).

As used herein, the term “alkynyl” refers to a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl. In some embodiments, alkynyl contains 2 to 20 carbon atoms, 2 to 16 carbon atoms, 2 to 12 carbon atoms, 2 to 8 carbon atoms, or 2 to 6 carbon atoms.

As used herein, the term “alkynylene” refers to a linking alkynyl group, i.e., an unsaturated, branched or straight chain or cyclic hydrocarbon radical having two monovalent radical centers derived by the removal of two hydrogen atoms from the same or two different carbon atoms of a parent alkyne. For example, an alkynylene group can have 2 to 20 carbon atoms, 2 to 16 carbon atoms, 2 to 12 carbon atoms, 2 to 8 carbon atoms, or 2 to 6 carbon atoms. Typical alkynylene radicals include, but are not limited to, acetylene (—C≡C—), propargyl (—CH₂C≡C—), and 4-pentynyl (—CH₂CH₂CH₂C≡C—).

As used herein, the term “amino” refers generally to a nitrogen radical which can be considered a derivative of ammonia, having the formula —N(X)₂, where each “X” is independently H, substituted or unsubstituted alkyl, substituted or unsubstituted carbocyclyl, substituted or unsubstituted heterocyclyl, etc. The hybridization of the nitrogen is approximately sp³. Nonlimiting types of amino include —NH₂, —N(alkyl)₂, —NH(alkyl), —N(carbocyclyl)₂, —NH(carbocyclyl), —N(heterocyclyl)₂, —NH(heterocyclyl), —N(aryl)₂, —NH(aryl), —N(alkyl)(aryl), —N(alkyl)(heterocyclyl), —N(carbocyclyl)(heterocyclyl), —N(aryl)(heteroaryl), —N(alkyl)(heteroaryl), etc. The term “alkylamino” refers to an amino group substituted with at least one alkyl group. Nonlimiting examples of amino groups include —NH₂, —NH(CH₃), —N(CH₃)₂, —NH(CH₂CH₃), —N(CH₂CH₃)₂, —NH(phenyl), —N(phenyl)₂, —NH(benzyl), —N(benzyl)₂, etc. Substituted alkylamino refers generally to alkylamino groups, as defined above, in which at least one substituted alkyl, as defined herein, is attached to the amino nitrogen atom. Non-limiting examples of substituted alkylamino includes —NH(alkylene-C(O)—OH), —NH(alkylene-C(O)—O-alkyl), —N(alkylene-C(O)—OH)₂, —N(alkylene-C(O)—O-alkyl)₂, etc.

As used herein, the term “aryl” refers to monocyclic or polycyclic (e.g., having 2, 3 or 4 fused rings) aromatic hydrocarbon radical derived by the removal of one hydrogen atom from a single carbon atom of a parent aromatic ring system. For example, an aryl group can include phenyl, naphthyl, anthracenyl, phenanthrenyl, indanyl, indenyl, and the like. In some embodiments, aryl groups have from 6 to about 20 carbon atoms, 6 to 14 carbon atoms, or 6 to 10 carbon atoms.

As used herein, the term “arylene” refers to a linking aryl group.

As used herein, the term “arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. The arylalkyl group can include 7 to 20 carbon atoms, e.g., the alkyl moiety is 1 to 6 carbon atoms and the aryl moiety is 6 to 14 carbon atoms.

As used herein, the term “arylalkenyl” refers to an acyclic alkenyl radical in which one of the hydrogen atoms bonded to a carbon atom, typically a terminal or sp³ carbon atom, but also an sp² carbon atom, is replaced with an aryl radical. The aryl portion of the arylalkenyl can include, for example, any of the aryl groups disclosed herein, and the alkenyl portion of the arylalkenyl can include, for example, any of the alkenyl groups disclosed herein. The arylalkenyl group can include 8 to 20 carbon atoms, e.g., the alkenyl moiety is 2 to 6 carbon atoms and the aryl moiety is 6 to 14 carbon atoms.

As used herein, the term “arylalkynyl” refers to an acyclic alkynyl radical in which one of the hydrogen atoms bonded to a carbon atom, typically a terminal or sp³ carbon atom, but also an sp carbon atom, is replaced with an aryl radical. The aryl portion of the arylalkynyl can include, for example, any of the aryl groups disclosed herein, and the alkynyl portion of the arylalkynyl can include, for example, any of the alkynyl groups disclosed herein. The arylalkynyl group can include 8 to 20 carbon atoms, e.g., the alkynyl moiety is 2 to 6 carbon atoms and the aryl moiety is 6 to 14 carbon atoms.

As used herein, the term “cycloalkyl” refers to non-aromatic cyclic hydrocarbons including cyclized alkyl, alkenyl, and alkynyl groups. Cycloalkyl groups can include mono- or polycyclic (e.g., having 2, 3 or 4 fused rings) ring systems as well as spiro ring systems. Ring-forming carbon atoms of a cycloalkyl group can be optionally substituted by oxo or sulfido. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcamyl, adamantyl, and the like. Also included in the definition of cycloalkyl are moieties that have one or more aromatic rings fused (i.e., having a bond in common with) to the cycloalkyl ring, for example, benzo or thienyl derivatives of pentane, pentene, hexane, and the like.

As used herein, the term “cycloalkylene” refers to a linking cycloalkyl group.

As used herein, the term “heteroalkyl” refers to an alkyl group having at least one heteroatom such as sulfur, oxygen, or nitrogen. For example, if the carbon atom of the alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, ahydroxyalkyl group (e.g., —CH₂CH₂—OH), an aminoalkyl group (e.g., —CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁-C₆ heteroalkyl group means a heteroalkyl group having 1 to 6 carbon atoms.

As used herein, the term “heteroalkylene” refers to a linking heteroalkyl group.

As used herein, the term “heteroaryl” refer to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, the term “heteroarylene” refers to a linking heteroaryl group.

As used herein, the term “alkoxy” refers to an —O-alkyl group. Example alkoxy groups include methoxy, ethoxy, propoxy (e.g., n-propoxy and isopropoxy), t-butoxy, and the like.

As used herein, the term “cycloalkoxy” refers to an —O-cycloalkyl group.

As used herein, the term “heterocycloalkoxy” refers to an —O-heterocycloalkyl group.

As used herein, the term “aryloxy” refers to an —O-aryl group. Example aryloxy groups include phenyl-O—, substituted phenyl-O—, and the like.

As used herein, the term “heteroaryloxy” refers to an —O-heteroaryl group.

As used herein, the term “arylalkyl” refers to alkyl substituted by aryl and “cycloalkylalkyl” refers to alkyl substituted by cycloalkyl. An example arylalkyl group is benzyl.

As used herein, the term “heteroarylalkyl” refers to alkyl substituted by heteroaryl and “heterocycloalkylalkyl” refers to alkyl substituted by heterocycloalkyl.

As used herein, the term “halo” or “halogen” includes fluoro, chloro, bromo, and iodo.

As used herein, the term “haloalkyl” refers to an alkyl group having one or more halogen substituents. Example haloalkyl groups include CF₃, C₂F₅, CHF₂, CCl₃, CHCl₂, C₂Cl₅, and the like.

As used herein, the term “haloalkenyl” refers to an alkenyl group having one or more halogen substituents.

As used herein, the term “haloalkynyl” refers to an alkynyl group having one or more halogen substituents.

As used herein, the term “haloalkoxy” refers to an —O-(haloalkyl) group.

As used herein, the term “ether” refers to a group including an oxygen atom connected to two alkyl or aryl groups. As used herein, a “vinyl ether” refers to an ether including a carbon-carbon double bond bound to the oxygen atom.

As used herein, the term “polyalkylene oxide” refers to non-ionic polymers including alkylene oxide monomers. Examples of polyalkylene oxides include polyethylene oxide (PEO), polypropylene oxide (PPO) and polyethylene glycol (PEG), or block copolymers including PO and/or PPO.

As used herein, the term “polysiloxane” refers to polymers having a backbone consisting essentially of alternate atoms of silicon and oxygen. The structures resulting from the silicon-oxygen-silicon linkages can be cyclic or straight-chain or branched-chain type.

As used herein, an “electron donating substituent” refers to a substituent that adds electron density to an adjacent pi (π)-system, making the π-system more nucleophilic. In some embodiments, an electron donating substituent has lone pair electrons on the atom adjacent to π-system. In some embodiments, electron donating substituents have π-electrons, which can donate electron density to the adjacent pi-system via hyperconjugation. Examples of electron donating substituents include O—, NR₂, NH₂, OH, OR, NHC(O)R, OC(O)R, aryl, and vinyl substituents.

As used herein, the term “unsaturated bond” refers to a carbon-carbon double bond or a carbon-carbon triple bond.

As used herein, the term “protecting group” refers to a moiety of a compound that masks or alters the properties of a functional group or the properties of the compound as a whole. The chemical substructure of a protecting group varies widely. One function of a protecting group is to serve as an intermediate in the synthesis of the parental drug substance. Chemical protecting groups and strategies for protection/deprotection are described, for example, in “Protective Groups in Organic Chemistry,” Theodora W. Greene (John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. “Hydroxy protecting groups” refers to those protecting groups useful for protecting hydroxy groups (—OH).

As used herein, “forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

As used herein, a “non-nucleophilic base” refers to an electron donor, a Lewis base, such as nitrogen bases including triethylamine, diisopropylethyl amine, N,N-diethylaniline, pyridine, 2,6-lutidine, 2,4,6-collidine, 4-dimethylaminopyridine, and quinuclidine.

As used herein, a “leaving group” refers to groups that maintain the bonding electron pair during heterolytic bond cleavage. For example, a leaving group is readily displaced during a displacement reaction. Suitable leaving groups include, but are not limited to, chloride, bromide, mesylate, tosylate, triflate, 4-nitrobenzenesulfonate, 4-chlorobenzenesulfonate, 4-nitrophenoxy, pentafluorophenoxy, etc. One of skill in the art will recognize other leaving groups useful in the present invention.

As used herein, a “deprotection agent” refers to any agent capable of removing a protecting group. The deprotection agent will depend on the type of protecting group used. Representative deprotection agents are known in the art and can be found in Protective Groups in Organic Chemistry, Peter G. M. Wuts and Theodora W. Greene, 4th Ed., 2006.

As used herein, a “luminophore” refers to a molecule having luminescent properties. Luminophores can be classified as fluorophores or phosphors, depending on the nature of the excited state responsible for the emission of photons, although certain luminophores (e.g., quantum dots, transition-metal complexes such as tris(bipyridine)ruthenium(II) chloride, whose luminescence comes from an excited (nominally triplet) metal-to-ligand charge-transfer (MLCT) state) cannot be classified as being exclusively fluorophores or phosphors. In some embodiments, luminophores include conjugated 11 systems. Luminophores can be organic or inorganic. In some embodiments, the luminophores of the present disclosure are organic molecules.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means +/−5%. As used herein, a recited ranges includes the end points, such that from 0.5 mole percent to 99.5 mole percent includes both 0.5 mole percent and 99.5 mole percent.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Luminescent Molecules

The present disclosure features a luminescent molecule, including a luminophore (e.g., a fluorescent dye); and a moiety including a heteroaryl core covalently and directly bonded to the luminophore, the heteroaryl core is optionally substituted with 1, 2, 3, or 4 substituents independently selected from C═O, amino, C₁₋₂₄ alkyl, C₂₋₂₄ alkenyl, C₂₋₂₄ alkynyl, poly(alkylene oxide), aryl, heteroaryl, a polysiloxane, and any combination thereof, wherein each substituent is optionally substituted with 1, 2, 3, or 4 substituents independently selected from C₁₋₂₄ alkyl, C₂₋₂₄ alkenyl, C₂₋₂₄ alkynyl, halo, SH, cyclic ether (e.g., epoxide), OH, CN, N₃, NCO, C(O)H, COOH, C(O)OR^{a}, C(O)NR^{a}, N═NR^{a}, aryl, heteroaryl, SO₃⁻, C—PO(OR^{a})(OR^{b}), NH₂, NHR^{a}, NR^{a}R^{b}, (NR^{a}R^{b}R^{c})⁺, wherein each of R^{a}, R^{b}, and R^{c} is independently H, alkyl, aryl, arylalkyl, or a heterocycle; wherein the luminescent molecule has an increased photoluminescence quantum yield relative to an analogous luminophore without a covalently bonded moiety including the heteroaryl core. The heteroaryl core can have at least one heteroatom ring member that is a N atom in the aromatic heterocycle.

In some embodiments, the luminescent molecule includes a luminophore; and a moiety including a purine core covalently and directly bonded to the luminophore, the purine core optionally substituted with 1, 2, 3, or 4 substituents independently selected from C═O, amino, C₁₋₂₄ alkyl, C₂₋₂₄ alkenyl, C₂₋₂₄ alkynyl, poly(alkylene oxide), aryl, heteroaryl, a polysiloxane, and any combination thereof, wherein each substituent is optionally substituted with 1, 2, 3, or 4 substituents independently selected from C₁₋₂₄ alkyl C₂₋₂₄ alkenyl, C₂₋₂₄ alkynyl, halo, SH, cyclic ether (e.g., epoxide), OH, CN, N₃, NCO, C(O)H, COOH, C(O)OR^{a}, C(O)NR^{a}, N═NR^{a}, aryl, heteroaryl, SO₃⁻, C—PO(OR^{a})(OR^{b}), NH₂, NHR^{a}, NR^{a}R^{b}, (NR^{a}R^{b}R^{c})⁺, wherein each of R^{a}, R^{b}, and R^{c} is independently H, alkyl, aryl, arylalkyl, or a heterocycle; wherein the luminescent molecule has an increased photoluminescence quantum yield relative to an analogous luminophore without a covalently bonded moiety including the purine core.

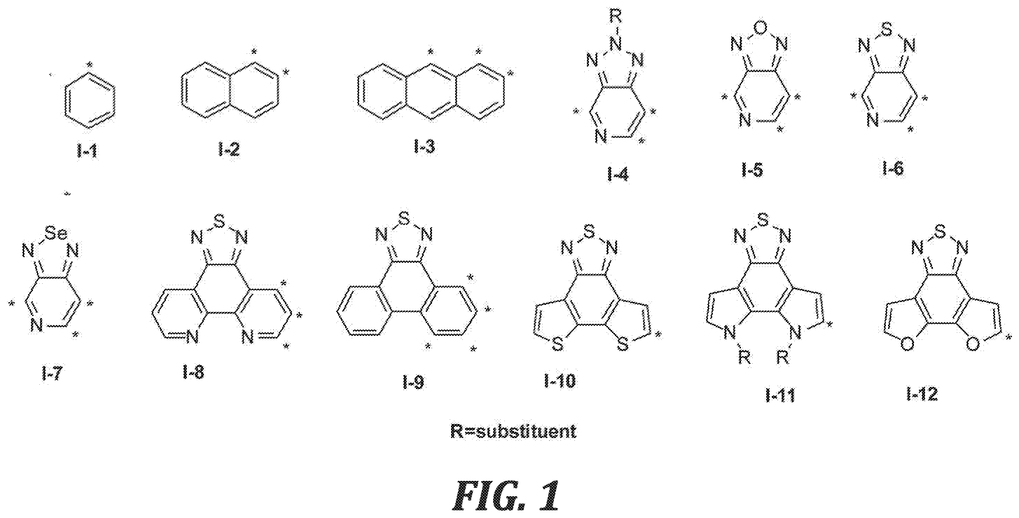

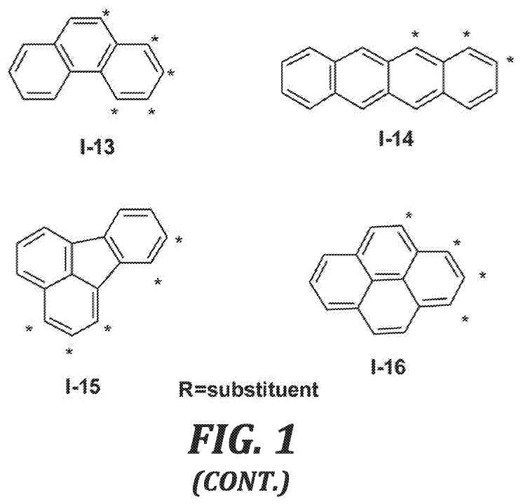

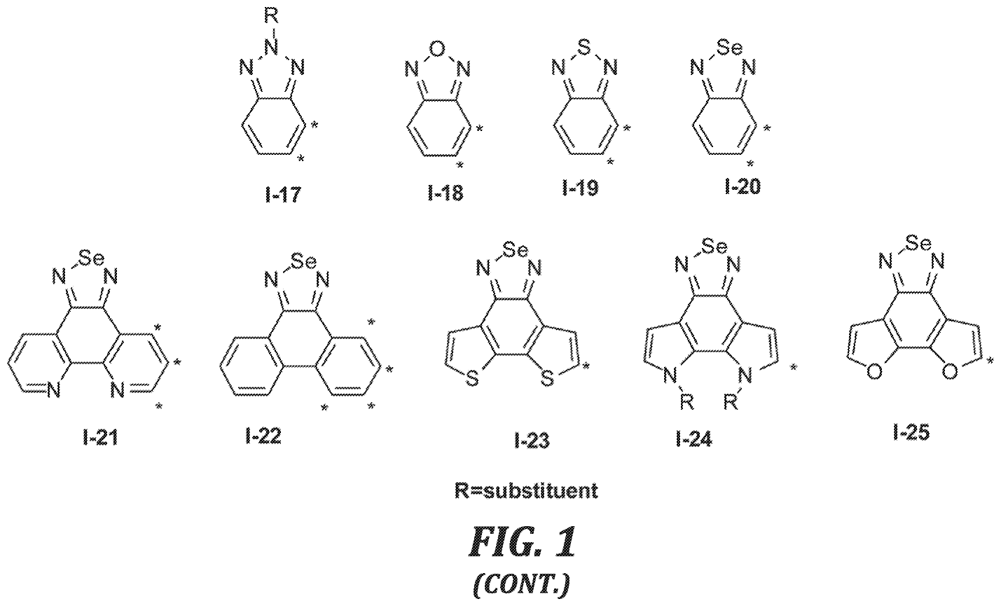

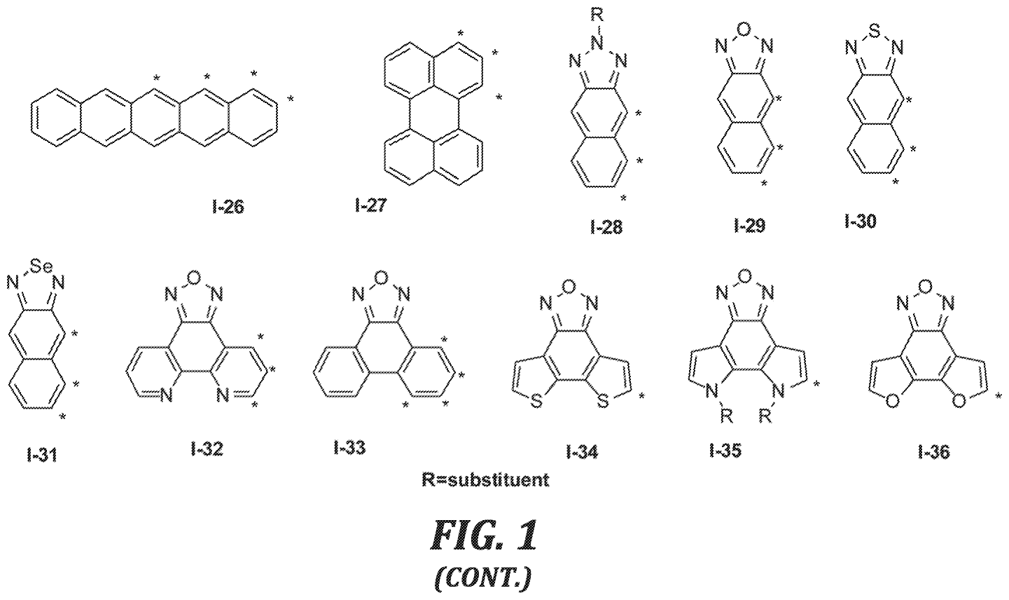

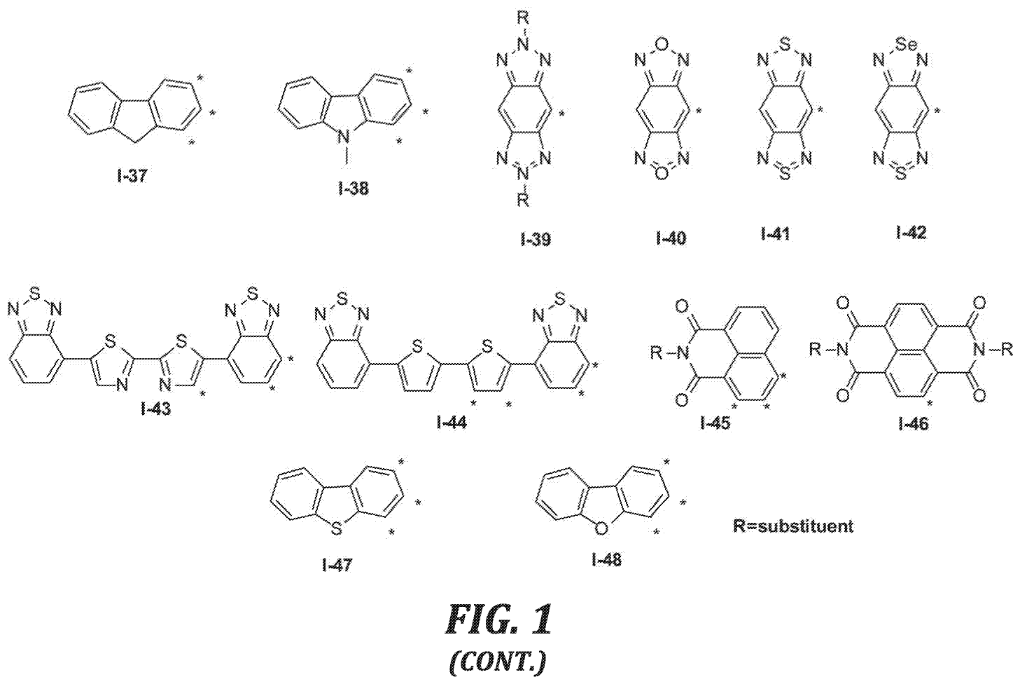

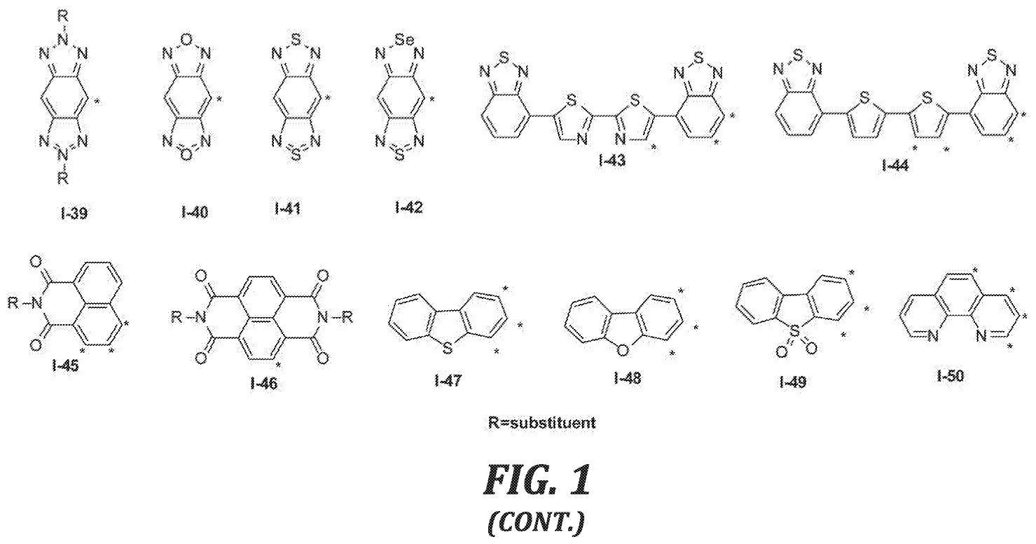

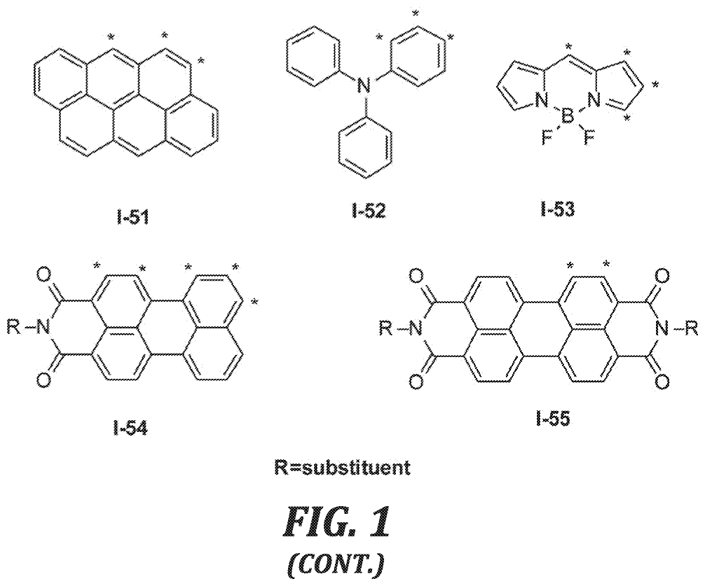

In some embodiments, the luminophore to which the moiety including the heteroaryl (e.g., purine) core can be attached is benzene, naphthalene, anthracene, phenanthrene, tetracene, fluoranthene, pyrene, pentacene, perylene, fluorene, carbazole, dibenzo[b,d]thiophene, dibenzo[b,d]furan, 1,10-phenanthroline, dibenzo[b,d]thiophene5,5-dioxide, [1,2,5]oxadiazolo[3,4-c]pyridine, [1,2,3]triazolo[4,5-c]pyridine, [1,2,5]selenadiazolo[3,4-c]pyridine, [1,2,5]thiadiazolo[3,4-c]pyridine, benzo[c][1,2,5]thiadiazole, benzo[c][1,2,5]oxadiazole, benzo[c][1,2,5]selenadiazole, naphtho[2,3-c][1,2,5]thiadiazole, naphtho[2,3-c][1,2,5]oxadiazole, naphtho[2,3-c][1,2,5]selenadiazole, benzo[d][1,2,3]triazole, naphtho[2,3-d][1,2,3]triazole, benzo[1,2-c:4,5-c′]bis[1,2,5]thiadiazole, benzo[1,2-c:4,5-c′]bis[1,2,5]oxadiazole, benzo[1,2-c:4,5-c′]bis[1,2,5]selenadiazole, benzo[1,2-c:4,5-c′] bis[1,2,5]triadiazole, triphenylamine, 5,5-difluoro-5H-414,514-dipyrrolo[1,2-c:2′,1′-f][1,3,2]diazaborinine, [1,2,5]thiadiazolo[3,4-f][1,10]phenanthroline, phenanthro[9,10-c][1,2,5]thiadiazole, dithieno[3′,2′:3,4;2″,3″:5,6]benzo[1,2-c][1,2,5]thiadiazole, 6,7-dihydropyrrolo[3,2-g][1,2,5]thiadiazolo[3,4-e]indole, furo[3′,2′:6,7]benzofuro[4,5-c][1,2,5]thiadiazole, [1,2,5]oxadiazolo[3,4-f][1,10]phenanthroline, phenanthro[9,10-c][1,2,5]oxadiazole, dithieno[3′,2′:3,4;2″,3″:5,6]benzo[1,2-c][1,2,5]oxadiazole, 6,7-dihydropyrrolo[3,2-g][1,2,5]oxadiazolo[3,4-e]indole, furo[3′,2′:6,7]benzofuro[4,5-c][1,2,5]oxadiazole, [1,2,5]selenadiazolo[3,4-f][1,10]phenanthroline, phenanthro[9,10-c][1,2,5]selenadiazole, dithieno[3′,2′:3,4;2″,3″:5,6]benzo[1,2-c][1,2,5]selenadiazole, 6,7-dihydropyrrolo[3,2-g][1,2,5]selenadiazolo[3,4-e]indole, furo[3′,2′:6,7]benzofuro[4,5-c][1,2,5]selenadiazole, 5,5′-bis(benzo[c][1,2,5]thiadiazol-4-yl)-2,2′-bithiazole, 5,5′-bis(benzo[c][1,2,5]thiadiazol-4-yl)-2,2′-bithiophene, benzo[de]isoquinoline-1,3(2H)-dione, benzo[lmn][3,8]phenanthroline-1,3,6,8(2H,7H)-tetraone, 2-methyl-1H-benzo[10,5]anthra[2,1,9-def]isoquinoline-1,3(2H)-dione, 2,9-dimethylanthra[2,1,9-def:6,5,10-d′e′f]diisoquinoline-1,3,8,10(2H,9H)-tetraone, naphtho[7,8,1,2,3-nopqr]tetraphene, or any combination thereof. Referring to FIG. 1, examples of luminophores are shown, with positions suitable for covalent bonding of the heteroaryl (e.g., purine) core indicated with *.

In some embodiments, the moiety including the heteroaryl (e.g., purine) core is a natural product or a derivative thereof. For example, the moiety including the purine core can include a purine moiety, a theobromine moiety, a caffeine moiety, a xanthine moiety, a guanine moiety, an isoguanine moiety, a paraxanthine moiety, an adenine moiety, a theophylline moiety, a hypoxanthine moiety, or any combination thereof.

The heteroaryl moiety can be a purine core of Formula (II):

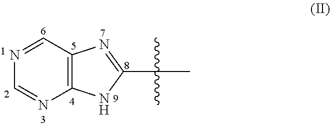 [SMILES: *c1nc2cncnc2[nH]1]

where positions 1, 2, 3, 6, 7, and 9 are each optionally substituted with a substituent independently selected from C═O, amino, C₁₋₂₄ alkyl, C₂₋₂₄ alkenyl, C₂₋₂₄ alkynyl, poly(alkylene oxide), aryl, heteroaryl, a polysiloxane, and any combination thereof, wherein each of amino, C₁₋₂₄ alkyl, C₂₋₂₄ alkenyl, C₂₋₂₄ alkynyl, poly(alkylene oxide), aryl, heteroaryl, or polysiloxane is optionally substituted with 1, 2, 3, or 4 substituents independently selected from C₁₋₂₄ alkyl, C₂₋₂₄ alkenyl, C₂₋₂₄ alkynyl, halo, SH, cyclic ether (e.g., epoxide), OH, CN, N₃, NCO, C(O)H, COOH, C(O)OR^{a}, C(O)NR^{a}, N═NR^{a}, aryl, heteroaryl, SO₃⁻, C—PO(OR^{a})(OR^{b}), NH₂, NHR^{a}, NR^{a}R^{b}, and (NR^{a}R^{b}R^{c})⁺, wherein each of R^{a}, R^{b}, and R^{c} is independently H, alkyl, aryl, arylalkyl, or a heterocycle, and wherein position 8 is covalently bound to the luminophore.

In some embodiments, the heteroaryl moiety is a purine core of Formula (II):

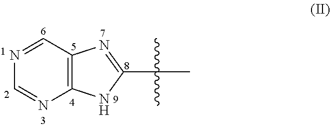 [SMILES: *c1nc2cncnc2[nH]1]

where positions 1, 2, 3, 6, 7, and 9 are each optionally substituted with a substituent independently selected from C═O, amino, C₁₋₂₄ alkyl, poly(alkylene oxide), aryl, or heteroaryl, wherein each of amino, C₁₋₂₄ alkyl, poly(alkylene oxide), aryl, or heteroaryl is optionally substituted with 1, 2, 3, or 4 substituents independently selected from C₁₋₂₄ alkyl, C₂₋₂₄ alkenyl, C₂₋₂₄ alkynyl, halo, OH, CN, N₃, NCO, C(O)H, COOH, C(O)OR^{a}, C(O)NR^{a}, aryl, heteroaryl, NH₂, NHR^{a}, and NR^{a}R^{b}, wherein each of R^{a}, R^{b}, and R^{c} is independently H or alkyl, and wherein position 8 is covalently bound to the luminophore.

In some embodiments, the heteroaryl moiety is a purine core of Formula (II):

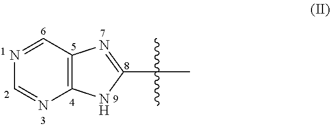 [SMILES: *c1nc2cncnc2[nH]1]

where positions 1, 2, 3, 6, 7, and 9 are each optionally substituted with a substituent independently selected from C═O, amino, C₁₋₂₄ alkyl, and poly(alkylene oxide).

In some embodiments, the heteroaryl moiety is a purine core of Formula (II):

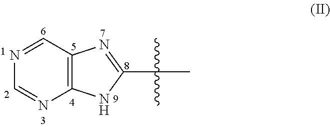 [SMILES: *c1nc2cncnc2[nH]1]

where positions 1, 2, 3, 6, 7, and 9 are each optionally substituted with a substituent independently selected from C═O, amino, and C₁₋₂₄ alkyl.

In some embodiments, the luminescent molecule's optical properties can be tuned by changing the N-substitution on the heteroaryl moiety (e.g., positions 1, 3, 7, and/or 9 on the purine core of Formula (II)). In some embodiments, the luminescent molecule's optical properties can be tuned by changing the N-substitution on the heteroaryl moiety (e.g., positions 1, 3, 7, and/or 9 on the purine core of Formula (II).

In some embodiments the luminescent molecule is

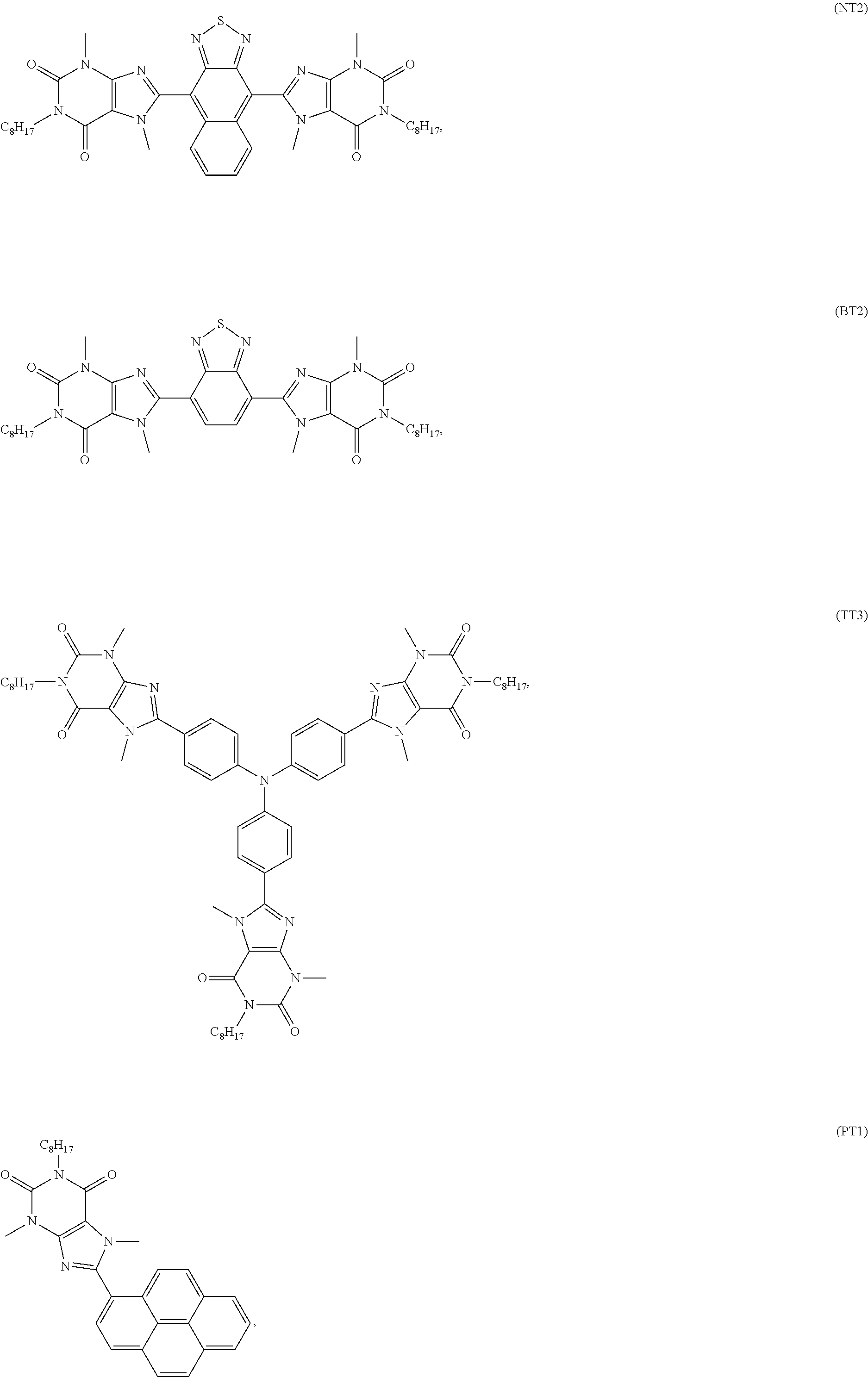

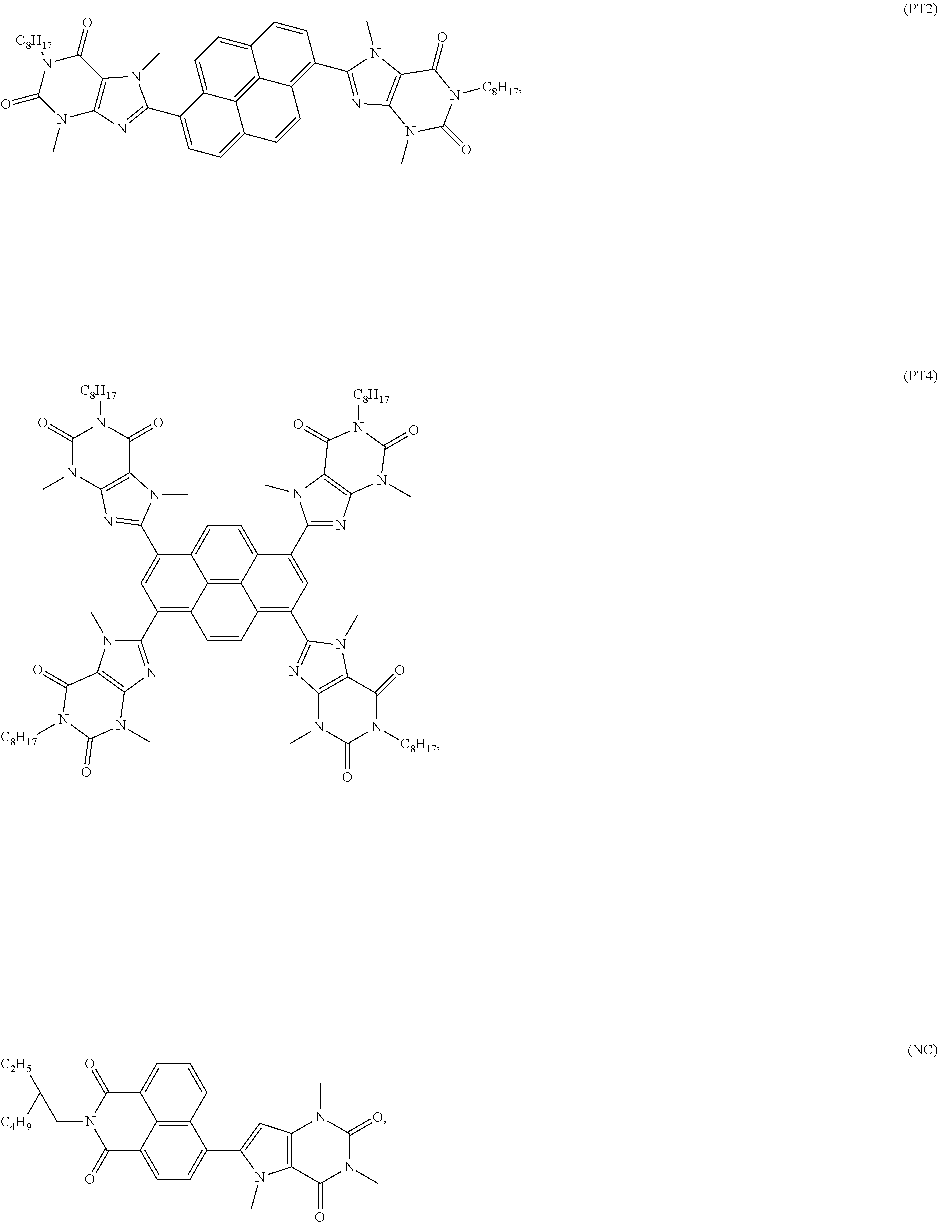

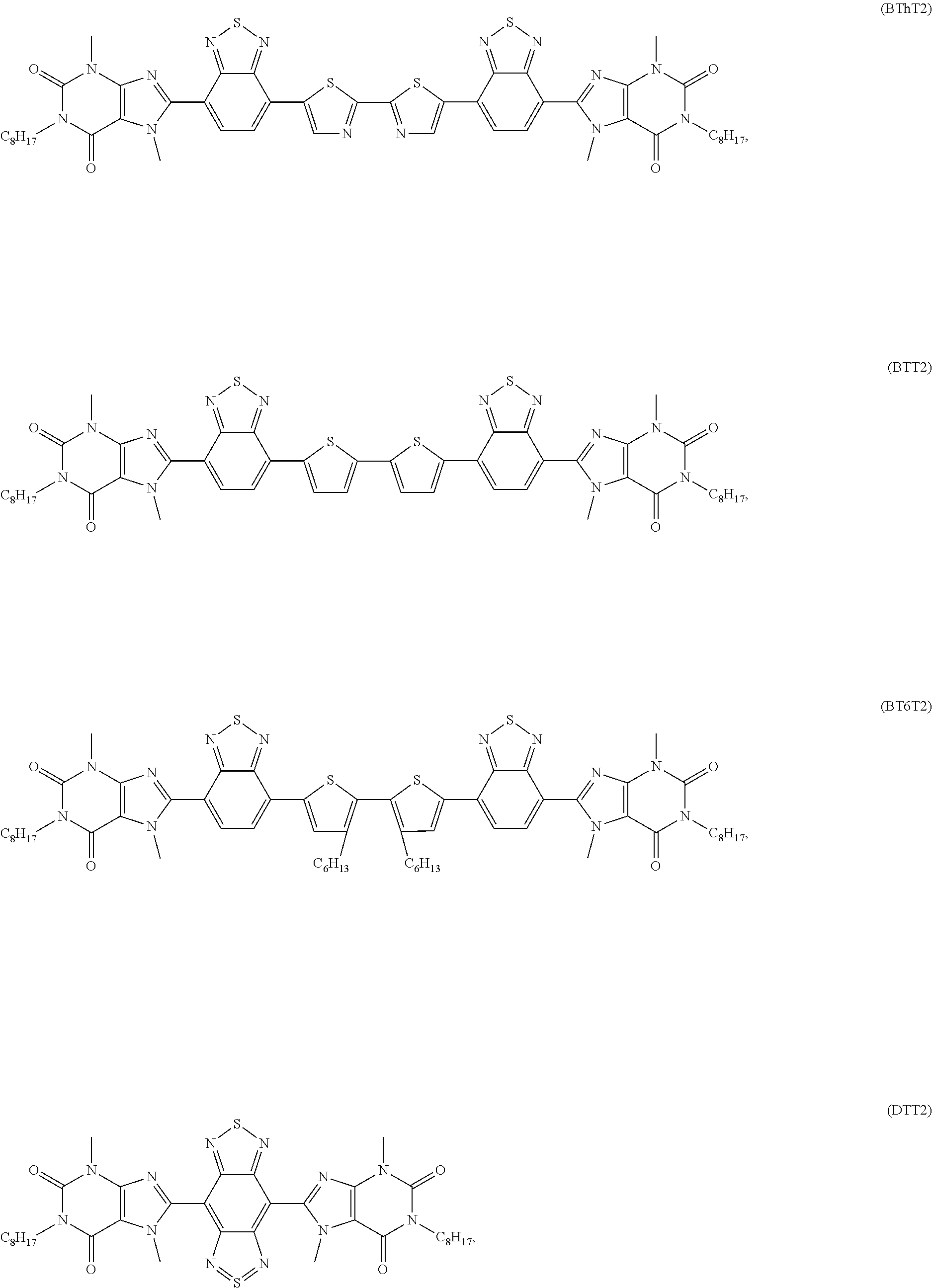

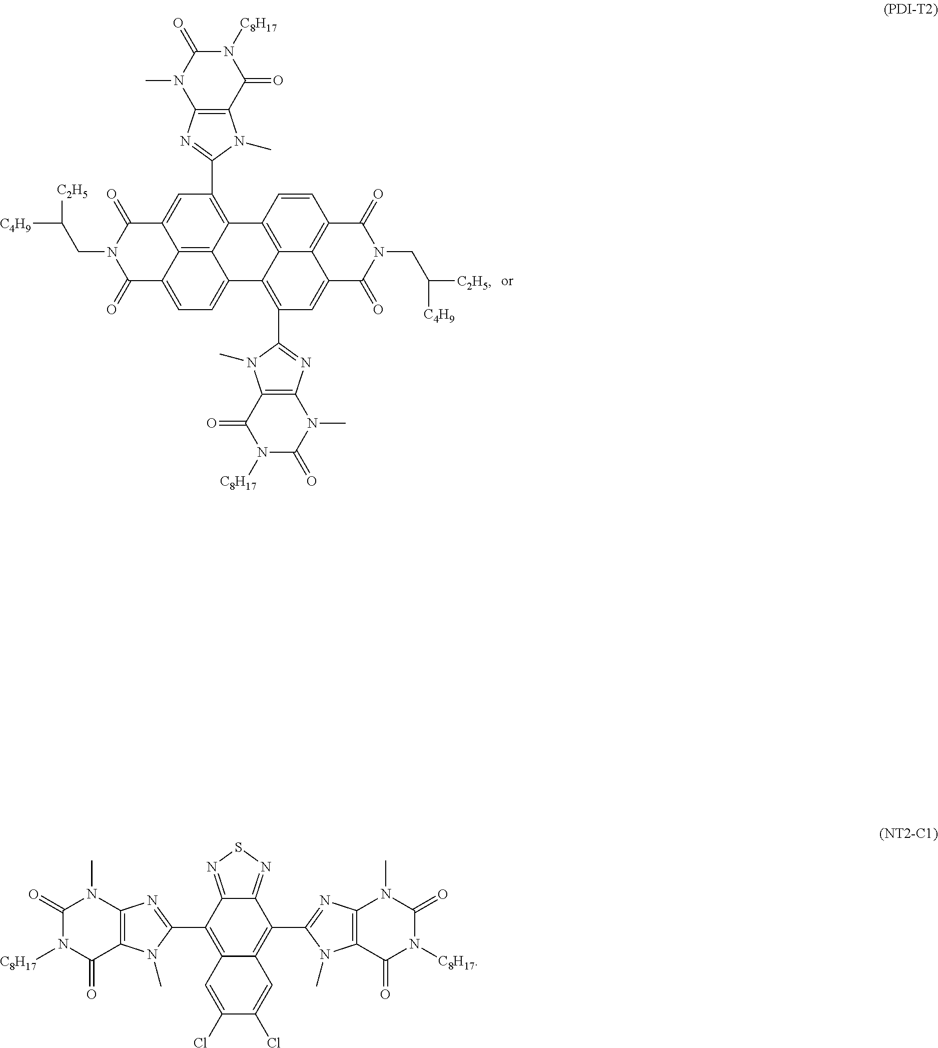

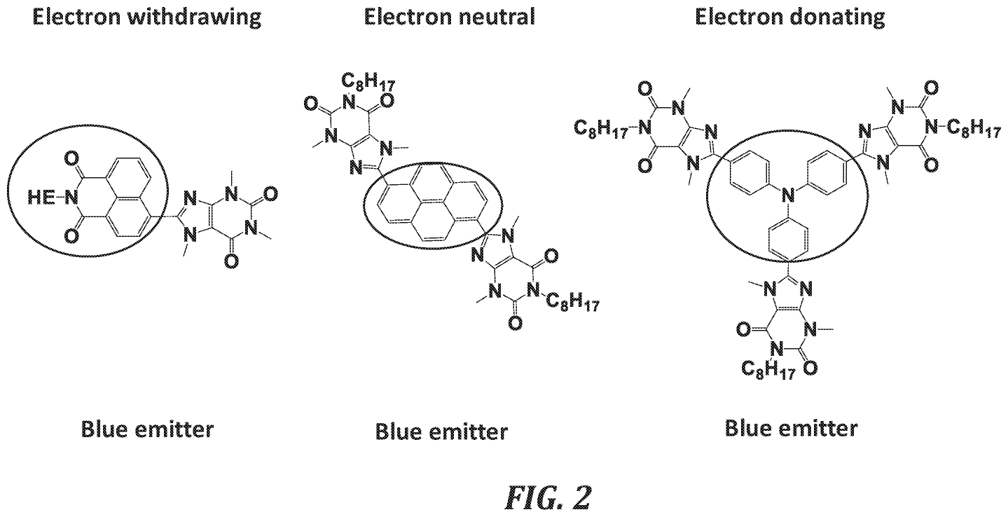

The luminescent molecules of the present disclosure can span a range of optioelectronic properties. For example, referring to FIG. 2, an exemplary moiety having a purine core can be covalently linked to various organic luminophores via position 8 of the purine core, with different electronic (electron-donating, electron-withdrawing and electron-neutral) and structural features ((hetero)aryl moiety and substituted (hetero)aryl moiety) on the luminophore, to provide luminescent molecules with tunable optoelectronic properties. Without wishing to be bound by theory, it is believed that certain moieties having a purine core, such as theobromine, are neither electron donors nor acceptors.

In some embodiments, the luminescent molecule has an increased photoluminescence quantum yield of at least 20% (e.g., at least 30%, at least 40%, at least 50%, at least 60%, at least 70%, at least 80%, at least 90%, or at least 95%, or at least 99%) relative to an analogous luminophore without a covalently bonded moiety including a heteroaryl (e.g., purine) core in a solid state. For example, the luminophore without a covalently bonded moiety including a heteroaryl core in a solid state can have a photoluminescence quantum yield of 50%, and the analogous luminescent molecule with a covalently bonded moiety including a heteroaryl core in a solid state can have a photoluminescence quantum yield of at least 70% (i.e., at least a 20% increase in photoluminescence quantum yield).

In some embodiments, the luminescent molecule has an increased photoluminescence quantum yield of at least 20% (e.g., at least 30%, at least 40%, at least 50%, at least 60%, at least 70%, at least 80%, at least 90%, or at least 95%, or at least 99%) relative to an analogous luminophore without a covalently bonded moiety including a heteroaryl (e.g., purine) core, when the luminescent molecule is in a concentration of at least 10⁻³ M (e.g., 10⁻² M, 10⁻¹ M, or 1M) in solution. In some embodiments, the luminescent molecule has an increased photoluminescence quantum yield of at least 20% (e.g., at least 30%, at least 40%, at least 50%, at least 60%, at least 70%, at least 80%, at least 90%, or at least 95%, or at least 99%) relative to an analogous luminophore without a covalently bonded purine moiety when the luminescent molecule is in a concentration of at least 10⁻³ M (e.g., 10⁻² M, 10⁻¹ M, or 1M) in a gel. In some embodiments, the luminescent molecule has decreased aggregation quenching relative to an analogous luminophore without a covalently bonded moiety including a heteroaryl (e.g., purine) core.

Synthesis Methods

The compounds of the present disclosure can be prepared in a variety of ways known to one skilled in the art of organic synthesis. The compounds of the present disclosure can be synthesized using the methods as hereinafter described below, together with synthetic methods known in the art of synthetic organic chemistry or variations thereon as appreciated by those skilled in the art.

The compounds of this disclosure can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like) HPLC, or preparative thin layer chromatography; distillation; sublimation, trituration, or recrystallization.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene's Protective Groups in Organic Synthesis, 4^{th} Ed., John Wiley & Sons: New York, 2006, which is incorporated herein by reference in its entirety.

The reactions of the processes described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the reaction step, suitable solvent(s) for that particular reaction step can be selected. Appropriate solvents include water, alkanes (such as pentanes, hexanes, heptanes, cyclohexane, etc., or a mixture thereof), aromatic solvents (such as benzene, toluene, xylene, etc.), alcohols (such as methanol, ethanol, isopropanol, etc.), ethers (such as dialkylethers, methyl tert-butyl ether (MTBE), tetrahydrofuran (THF), dioxane, etc.), esters (such as ethyl acetate, butyl acetate, etc.), halogenated solvents (such as dichloromethane (DCM), chloroform, dichloroethane, tetrachloroethane), dimethylformamide (DMF), dimethylsulfoxide (DMSO), acetone, acetonitrile (ACN), hexamethylphosphoramide (HMPA) and N-methylpyrrolidone (NMP). Such solvents can be used in either their wet or anhydrous forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

The luminescent molecules of the disclosure can be prepared, for example, using the reaction pathways and techniques as described below.

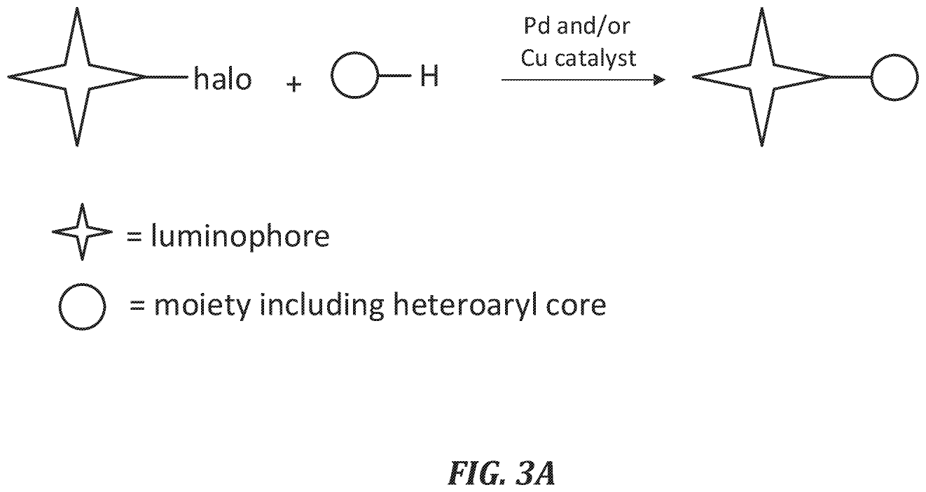

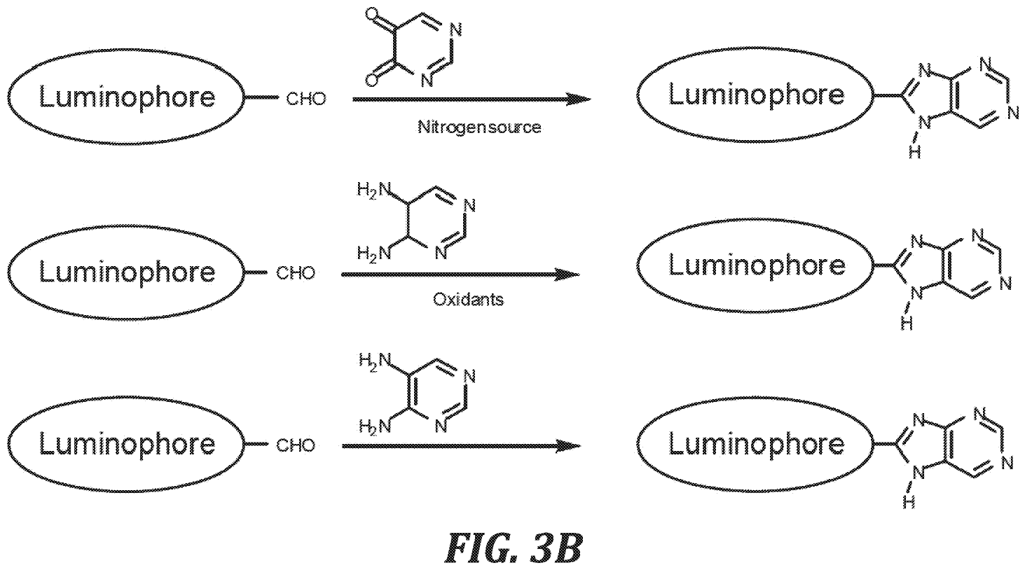

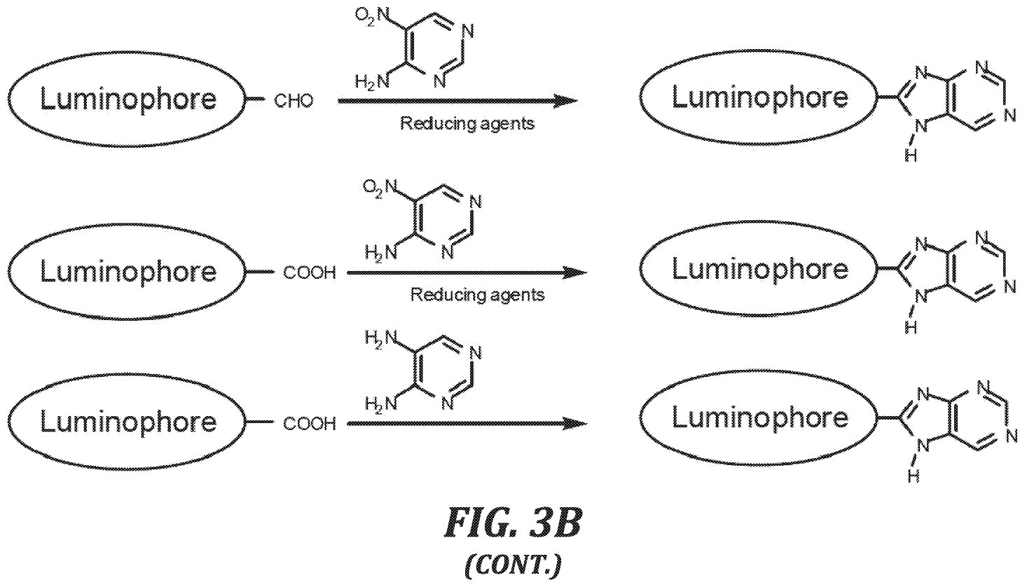

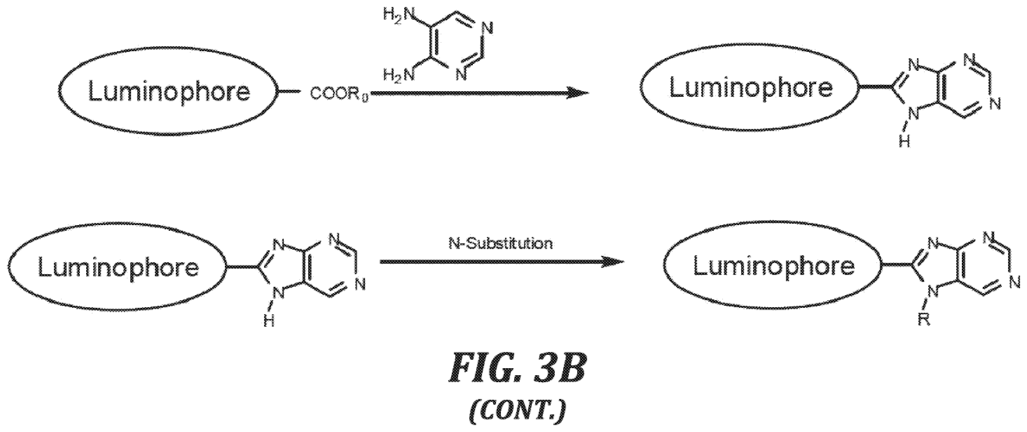

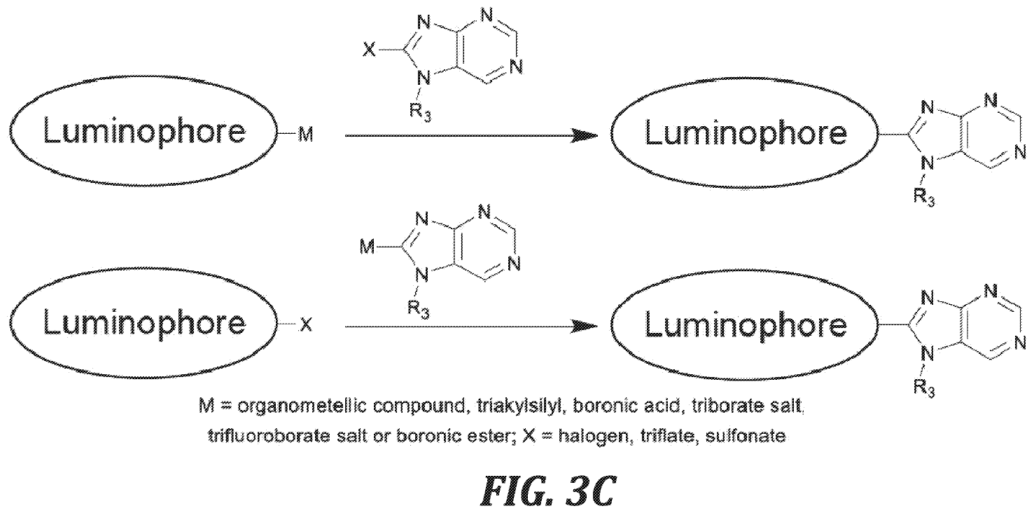

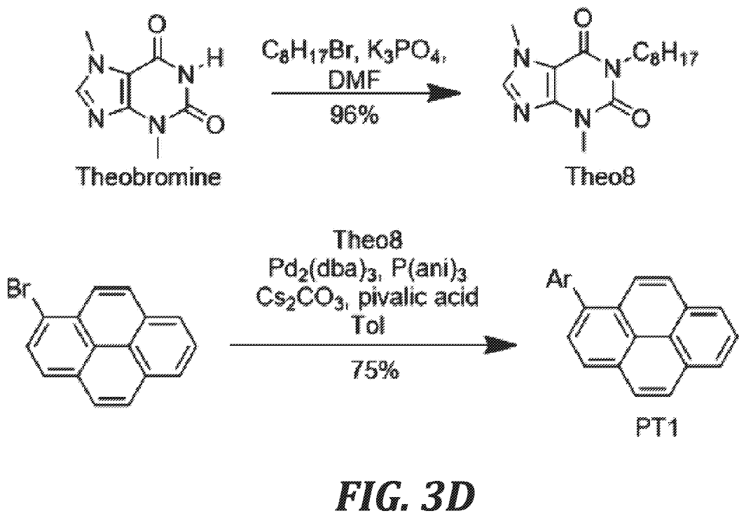

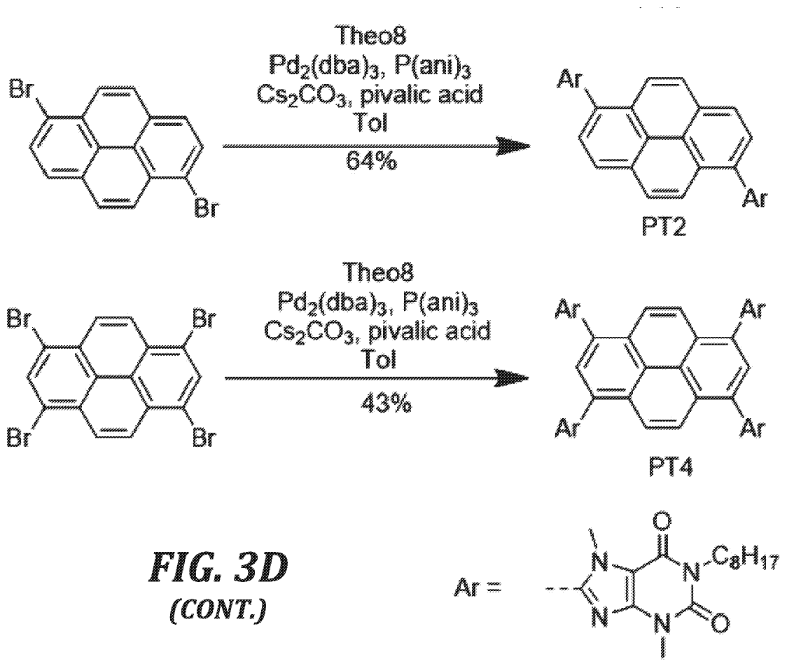

Referring to FIG. 3A, the present disclosure features methods of making the above-described luminescent molecules, including: providing a reaction mixture including a luminophore including a leaving group covalently bound thereto, a compound including a heteroaryl (e.g., purine) core and an activatable C—H bond, and a palladium or copper catalyst; activating the activatable C—H bond of the compound including the heteroaryl (e.g., purine) core with the palladium or copper catalyst to provide an activated compound including the heteroaryl (e.g., purine) core, and reacting the luminophore including a leaving group covalently bound thereto with the activated compound including the heteroaryl (e.g., purine) core to provide the above-mentioned luminescent molecules. In some embodiments, the heteroaryl substituent of the luminescent molecules can be N-substituted post-synthesis. In certain embodiments, the heteroaryl substituent is N-substituted, then reacted with the luminophore to provide a luminescent molecule.

In some embodiments, the leaving group covalently bound to the luminophore is halo (e.g., iodo, bromo, chloro), tosylate, mesylate, triflate, 4-nitrobenzenesulfonate, 4-chlorobenzenesulfonate, 4-nitrophenoxy, pentafluorophenoxy, and the like. In some embodiments, the leaving group covalently bound to the luminophore is halo, triflate, tosylate, or mesylate. In certain embodiments, the leaving group covalently bound to the luminophore is halo. The luminophore including a leaving group covalently bound thereto can include 1, 2, 3, or 4 leaving groups (e.g., 1, 2, or 3 leaving groups; 1 or 2 leaving groups; or 1 leaving group).

In some embodiments, the methods of making the luminescent molecule are environmentally-friendly and scalable. The methods include covalently attaching an moiety including a heteroaryl (e.g., purine) core onto an organic luminophore via direct arylation and allowing direct cross-coupling between the moiety including the heteroaryl core and the organic luminophore to provide a luminescent molecule. The luminescent molecule has decreased aggregation caused quenching compared to a luminescent molecule without the moiety including the heteroaryl core (e.g., a xanthine derivative moiety, a functionalized xanthine derivative moiety, a C₃₋₂₀ alkyl-functionalized xanthine derivative moiety). In some embodiments, the heteroaryl substituent of the luminescent molecules is be N-substituted post-synthesis.

In certain embodiments, the methods include alkylating a moiety having a heteroaryl core (e.g., purine core of structure (II), such as a xanthine or a theobromine), covalently attaching the alkylated moiety having the heteroaryl core, activating a C—H bond on the alkylated moiety having the heteroaryl core, covalently bonding the alkylated moiety having the heteroaryl core onto a luminophore (e.g., an organic luminophore) including a leaving group covalently bound thereto via a direct arylation reaction to provide the luminescent molecule of the present disclosure. For example, a synthesis of a pyrene having a directly covalently bonded alkylated theobromine moiety (or multiple alkylated theobromine moieties) is shown in FIG. 3D.

In some embodiments, referring to FIGS. 3B and 3C, alternative synthetic routes are used to form the luminescent molecules of the present disclosure and the heteroaryl substituent on the luminescent molecules can be N-substituted post-synthesis. While one moiety including a heteroaryl core is depicted in the final product, it is understood that the luminophore can be covalently bound to multiple moieties including a heteroaryl core. As shown in FIG. 3B, the heteroaryl moiety can be generated during the synthesis of luminescent molecule, by a ring-forming reaction with a luminophore having an aldehyde, carboxylic acid, or ester group. Post-synthesis N-substitution of the heteroaryl substituent can be carried out by, for example, by reacting the luminescent molecule in the presence of base (e.g., NaH, K₃PO₄, Cs₂CO₃, K₂CO₃, Na₂CO₃, sodium acetate, potassium acetate, and/or cesium acetate, NaOH, KOH, sodium methoxide, potassium methoxide, sodium ethoxide, potassium ethoxide, sodium isopropoxide, potassium isopropoxide sodium tert-butoxide, and potassium tert-butoxide, etc.) to deprotonate an —NH group on the purine moiety, followed by reacting the deprotonated luminescent molecule with a reactant having a leaving group to provide a N-substituted luminescent molecule. As shown in FIG. 3C, alternative synthetic routes to the luminescent molecules of the present disclosure can include cross-coupling reactions with pre-functionalized purine moieties (halo/metallic/boronic precursors), and the heteroaryl substituent on the luminescent molecules can be N-substituted before cross-coupling and/or post-synthesis of the luminescent molecule.

The luminophore including a leaving group covalently bound thereto to which the moiety including the heteroaryl (e.g., purine) core can be attached can include a benzene, naphthalene, anthracene, phenanthrene, tetracene, fluoranthene, pyrene, pentacene, perylene, fluorene, carbazole, dibenzo[b,d]thiophene, dibenzo[b,d]furan, 1,10-phenanthroline, dibenzo[b,d]thiophene5,5-dioxide, [1,2,5]oxadiazolo[3,4-c]pyridine, [1,2,3]triazolo[4,5-c]pyridine, [1,2,5]selenadiazolo[3,4-c]pyridine, [1,2,5]thiadiazolo[3,4-c]pyridine, benzo[c][1,2,5]thiadiazole, benzo[c][1,2,5]oxadiazole, benzo[c][1,2,5]selenadiazole, naphtho[2,3-c][1,2,5]thiadiazole, naphtho[2,3-c][1,2,5]oxadiazole, naphtho[2,3-c][1,2,5]selenadiazole, benzo[d][1,2,3]triazole, naphtho[2,3-d][1,2,3]triazole, benzo[1,2-c:4,5-c′]bis[1,2,5]thiadiazole, benzo[1,2-c:4,5-c′]bis[1,2,5]oxadiazole, benzo[1,2-c:4,5-c′]bis[1,2,5]selenadiazole, benzo[1,2-c:4,5-c′]bis[1,2,5]triadiazole, triphenylamine, 5,5-difluoro-5H-414,514-dipyrrolo[1,2-c:2′,1′-f][1,3,2]diazaborinine, [1,2,5]thiadiazolo[3,4-f][1,10]phenanthroline, phenanthro[9,10-c][1,2,5]thiadiazole, dithieno[3′,2′:3,4;2″,3″:5,6]benzo[1,2-c][1,2,5]thiadiazole, 6,7-dihydropyrrolo[3,2-g][1,2,5]thiadiazolo[3,4-e]indole, furo[3′,2′:6,7]benzofuro[4,5-c][1,2,5]thiadiazole, [1,2,5]oxadiazolo[3,4-f][1,10]phenanthroline, phenanthro[9,10-c][1,2,5]oxadiazole, dithieno[3′,2′:3,4;2″,3″:5,6]benzo[1,2-c][1,2,5]oxadiazole, 6,7-dihydropyrrolo[3,2-g][1,2,5]oxadiazolo[3,4-e]indole, furo[3′,2′:6,7]benzofuro[4,5-c][1,2,5]oxadiazole, [1,2,5]selenadiazolo[3,4-f][1,10]phenanthroline, phenanthro[9,10-c][1,2,5] selenadiazole, dithieno[3′,2′:3,4;2″,3″:5,6]benzo[1,2-c][1,2,5]selenadiazole, 6,7-dihydropyrrolo[3,2-g][1,2,5]selenadiazolo[3,4-e]indole, furo[3′,2′:6,7]benzofuro[4,5-c][1,2,5]selenadiazole, 5,5′-bis(benzo[c][1,2,5]thiadiazol-4-yl)-2,2′-bithiazole, 5,5′-bis(benzo[c][1,2,5]thiadiazol-4-yl)-2,2′-bithiophene, benzo[de]isoquinoline-1,3(2H)-dione, benzo[lmn][3,8]phenanthroline-1,3,6,8(2H,7H)-tetraone, 2-methyl-1H-benzo[10,5]anthra[2,1,9-def]isoquinoline-1,3(2H)-dione, 2,9-dimethylanthra[2,1,9-def:6,5,10-d′e′f]diisoquinoline-1,3,8,10(2H,9H)-tetraone, naphtho[7,8,1,2,3-nopqr]tetraphene, or any combination thereof, each including a leaving group covalently bound thereto. Referring to FIG. 1, examples of luminophores are shown, with positions suitable for leaving group substitution of the luminophore indicated with *. In some embodiments, the leaving group on the luminophore is reactive and is selected from chloro, bromo, and iodo. The luminophore including a leaving group covalently bound thereto can include 1, 2, 3, or 4 leaving groups (e.g., 1, 2, or 3 leaving groups; 1 or 2 leaving groups; or 1 leaving group). The luminophore having leaving group covalently bound thereto can react with a moiety including a heteroaryl core having an activated C—H bond, where the leaving group is replaced with a heteroaryl group, such as a structure of Formula (II), via a direct arylation reaction, to provide the luminescent molecule. In some embodiments, the leaving group covalently bound to the luminophore is halo (e.g., iodo, bromo, chloro), tosylate, mesylate, triflate, 4-nitrobenzenesulfonate, 4-chlorobenzenesulfonate, 4-nitrophenoxy, pentafluorophenoxy, or the like. In some embodiments, the leaving group covalently bound to the luminophore is halo, triflate, tosylate, or mesylate. In certain embodiments, the leaving group covalently bound to the luminophore is halo.

In some embodiments, the palladium catalyst is present in the reaction mixture in an amount of 0.01M to 0.2M (e.g., 0.01M to 0.15M, 0.01M to 0.05M, or 0.01M to 0.03M) relative to the luminophore including a leaving group covalently bound thereto.

Examples of palladium catalysts include, without limitation, Pd₂(dba)₃, bis[5-(di(1-adamantyl)phosphino)-1′,3′,5′-triphenyl-1′H-[1,4′]bipyrazole] palladium(II) dichloride, [2-(di-1-adamantylphosphino)-2′,4′,6′-triisopropyl-3,6-dimethoxybiphenyl][2-(2′-amino-1,1′-biphenyl)]palladium(II) methanesulfonate, allyl[1,3-bis(2,6-diisopropylphenyl)-2-imidazolidinylidene]chloropalladium(II), allyl[1,3-bis(2,6-diisopropylphenyl)imidazol-2-ylidene] chloropalladium(II), allyl[1,3-bis(mesityl)imidazol-2-ylidene]chloropalladium(II), allylpalladium(II) chloride dimer; chloro[4-(di-tert-butylphosphino)-N,N-dimethylaniline-2-(2′-aminobiphenyl)]palladium(II), [4-(di-tert-butylphosphino)-N,N-dimethylaniline-2-(2′-aminobiphenyl)]palladium(II) methanesulfonate, APhos Pd G4, trans-benzyl(chloro)bis(triphenylphosphine)palladium(II), (bicyclo[2.2.1]hepta-2,5-diene)dichloropalladium(II), rac-BINAP-Pd-G3, (2,2′-bipyridine)dichloropalladium(II), trans-bis(acetato)bis[o-(di-o-tolylphosphino)benzyl] dipalladium(II), bis(acetonitrile)dichloropalladium(II), bis(benzonitrile)palladium(II) bromide, bis(benzonitrile)palladium(II) chloride, bis[1,2-bis(diphenylphosphino)ethane]palladium(0), bis(dibenzylideneacetone)palladium(0), bis(di-tert-butyl(4-dimethylaminophenyl)phosphine) dichloropalladium(II), [1,1′-bis(di-tertbutylphosphino)ferrocene]dichloropalladium(II), bis[di-(tert-butyl)(4-trifluoromethylphenyl)phosphine] palladium(II) chloride, trans-bis(dicyclohexylamine)palladium(II) acetate, bis[(dicyclohexyl)(4-dimethylaminophenyl)phosphine] palladium(II) chloride, [1,1′-bis(dicyclohexylphosphino)ferrocene]dichloropalladium(II), [1,3-bis(2,6-di-isopropylphenyl)-4,5-dihydroimidazol-2-ylidene]chloro[3-phenylallyl]palladium(II), [1,3-bis(2,6-diisopropylphenyl)imidazol-2-ylidene]chloro[3-phenylallyl]palladium(II), 1,3-bis(2,6-diisopropylphenyl)imidazol-2-ylidene(1,4-naphthoquinone)palladium(0) dimer, 1,1′-bis(di-isopropylphosphino)ferrocene palladium dichloride, bis(3,5,3′,5′-dimethoxydibenzylideneacetone)palladium(0), bis(N,N-dimethyl-)-alaninato)palladium(II), 1,4-bis(diphenylphosphino)butane-palladium(II) chloride, [1,1′-bis(diphenylphosphino)ferrocene]dichloropalladium(II), bis[1,2-bis(diphenylphosphino)ethane]palladium(0), (1,3-bis(diphenylphosphino)propane)palladium(II) chloride, [1,3-bis(diphenylphosphino)propane]palladium(II) triflate, [2,6-bis[(di-1-piperidinylphosphino)amino]phenyl]palladium(II) chloride, 1,2-bis(phenylsulfinyl)ethane palladium(II) acetate, bis(tri-tert-butylphosphine)palladium(0), bis(tricyclohexylphosphine)palladium(0), 1,3-bis(2,4,6-trimethylphenyl)imidazol-2-ylidene (1,4-naphthoquinone)palladium(0) dimer, bis(triphenylphosphine)palladium(II) diacetate, bis(triphenylphosphine)palladium(II) dichloride, bis[tris(2-methylphenyl)phosphine]palladium, bis[tris(3-(1H,1H,2H-perfluorodecyl)phenyl)phosphine]palladium(II) dichloride, BrettPhos Pd G1 methyl t-butyl ether: trans-bromo(N-succinimidyl)bis(triphenylphosphine)palladium(II), tBuDavePhos Pd G3, t-BuDavePhos Pd G4, P(t-Bu)₃ Pd G2, (t-Bu)PhCPhos Pd G4, (2-butenyl)chloropalladium dimer, [(η3-1-tert-butylindenyl)(μ-Cl)Pd]2, tBuXPhos Pd G1, chloro{2,6-bis[(phenylseleno-Se)methyl]phenyl-C}palladium(II), chloro(1-tert-butyl-1H-inden-1-yl)(tri-tert-butylphosphine)palladium, chloro(1,5-cyclooctadiene)methylpalladium(II), chlorophenylallyl[1,3-bis(2,6-diphenylmethyl)-4-methoxyphenylimidazol-2-ylidene]palladium(II), chlorophenylallyl[1,3-bis(2,6-diphenylmethyl)-4-methylphenyl-2-imidazolylidene]palladium(II), chloro(η2-P,C-tris(2,4-di-tert-butylphenyl)phosphite)(tricyclohexylphosphine)palladitum(II), CPhos Pd G2, CPhos Pd G3, CPhos Pd G4, (η5-2,4-cyclopentadien-1-yl)[(1,2,3-η)-1-phenyl-2-propenyl]-palladium, CyJohnPhos Pd G2, CyJohnPhos Pd G3, DavePhos Pd G2, DavePhos-Pd-G3, diacetatobis(tricyclohexylphosphine)palladium(1), dibromo[2,2′-bis(diphenylphosphino)-1,1′-binaphthyl]palladium(II), dibromo[1,1′-bis(diphenylphosphino)ferrocene]palladium(II), trans-dibromobis(triphenylphosphine)palladium(II), trans-dibromo[bis(tri-o-tolylphosphine)]palladium(II), dibromo(1,5-cyclooctadiene)palladium(II), 2-(2′-di-tert-butylphosphine)biphenylpalladium(II) acetate, 8-(di-tert-butylphosphinooxy)quinoline, di-chlorobis[2′-(amino-N)[1,1′-biphenyl]-2-yl-C]dipalladium(1), dichlorobis[(tert-butyl)dicyclohexylphosphine]palladium(II), di-μ-chlorobis[5-chloro-2-[(4-chlorophenyl)(hydroxyimino-κN)methyl]phenyl-κC]palladium dimer, dichlorobis(chlorodi-tert-butylphosphine) palladium (II), dichlorobis[cyclohexyldi(1-piperidinyl)phosphine]palladium(II), dichlorobis(di-tert-butylphenylphosphine)palladium(II), dichlorobis(dicyclohexyl-1-piperidinylphosphine)palladium(II), dichloro[1,3-bis(2,6-diisopropylphenyl)-1,3-dihydro-2H-imidazol-2-ylidene]palladium(II) dimer, dichloro-[1,3-bis(diisopropylphenyl)imidazoliden-2-ylidene]palladium(II) dimer, di-μ-chlorobis[1-[(1R)-1-(dimethylamino)ethyl]-2-naphthyl-C,N]dipalladium(II), di-μ-chlorobis[2-[(dimethylamino)methyl]phenyl-C,N]dipalladium(II), dichloro[2,2′-bis(diphenylphosphino)-1,1′-binaphthyl]palladium(II), dichloro[1,1′-bis(diphenylphosphino)ferrocene]palladium(II)acetone adduct, di-μ-chlorobis[5-hydroxy-2-[1-(hydroxyimino-κN)ethyl]phenyl-κC]palladium(II) dimer, dichlorobis(methyldiphenylphosphine)palladium(II), dichlorobis(tricyclohexylphosphine)palladium(II), dibromo(1,5-cyclooctadiene)palladium(II), 2-(2′-di-tert-butylphosphine)biphenylpalladium(II) acetate, 8-(di-tert-butylphosphinooxy)quinoline, di-μ-chlorobis[2′-(amino-N)(1,1′-biphenyl]-2-yl-C]dipalladium(II), dichlorobis[(tert-butyl)dicyclohexylphosphine]palladium(II), di-μ-chlorobis[5-chloro-2-[(4-chlorophenyl)(hydroxyimino-κN)methyl]phenyl-KC]palladium dimer, dichlorobis(chlorodi-tert-butylphosphine) palladium (ii), dichlorobis[cyclohexyldi(1-piperidinyl)phosphine]palladium(II), dichlorobis(di-tert-butylphenylphosphine)palladium(II), dichlorobis(dicyclohexyl-1-piperidinylphosphine)palladium(II), dichloro[1,3-bis(2,6-diisopropylphenyl)-1,3-dihydro-2H-imidazol-2-ylidene] palladium(II) dimer, dichloro-[1,3-bis(diisopropylphenyl)imidazoliden-2-ylidene]palladium(II) dimer, di-μ-chlorobis[l-[(1R)-1-(dimethylamino)ethyl]-2-naphthyl-C,N]dipalladium(II), di-μ-chlorobis[2-[(dimethylamino)methyl]phenyl-C,N]dipalladium(II), dichloro[2,2′-bis(diphenylphosphino)-1,1′-binaphthyl]palladium(II), dichloro[1,1′-bis(diphenylphosphino)ferrocene]palladium(II)acetone adduct, di-μ-chlorobis[5-hydroxy-2-[1-(hydroxyimino-κN)ethyl]phenyl-KC]palladium(II) dimer, dichlorobis(methyldiphenylphosphine)palladium(II), dichlorobis(tricyclohexylphosphine)palladium(II), dichlorobis(triethylphosphine)palladium(II), dichlorobis[tri(1-piperidinyl)phosphine]palladium(II), dichlorobis(tri-o-tolylphosphine)palladium(II), dichloro(1,5-cyclooctadiene)palladium(II), dichloro[8-(di-tert-butylphosphinooxy)quinoline]palladium(II), dichloro[2-(4,5-dihydro-2-oxazolyl)quinoline]palladium(II), dichloro[9,9-dimethyl-4,5-bis(diphenylphosphino)xanthene]palladium(II), di-μ-chlorodimethylbis(triphenylphosphine)dipalladium, dichloro(1,10-phenanthroline)palladium(II) dichloro(N,N,N′,N′-tetramethylethylenediamine)palladium(II), 2,6-Difluoroanilino(oxo)acetic acid, dihydrogen dichlorobis(di-t-butylphosphinito-kp)palladate(²⁻), 2′-(dimethylamino)-2-biphenylyl-palladium(II) chloride dinorbornylphosphine complex, 2-(dimethylaminomethyl)ferrocen-1-yl-palladium(II) chloride dinorbornylphosphine complex, 2,6-dimethylanilino(oxo)acetic acid, [1-(diphenylphosphino)ethyl]ferrocene, DTBPF-Pd-G3, (ethylenediamine)palladium(II) chloride, JackiePhos Pd G3, Josiphos SL-J009-1 Pd G3, (2-methylallyl)palladium(II) chloride dimer, 2,3,7,8,12,13,17,18-octaethyl-21H,23H-porphine palladium(II), palladium(II) acetate, Palladium(II) acetylacetonate, palladium(II)[1,3-bis(diphenylphosphino)propane]-bis(benzonitrile)-bis-tetrafluoroborate, palladium(II) bromide, palladium(II) chloride ReagentPlus®, palladium(II) chloride, palladium(π-cinnamyl) chloride dimer, palladium(II) cyanide, palladium(II) hexafluoroacetylacetonate, palladium(II) iodide, palladium(II) nitrate dihydrate, palladium(II) oxide, palladium(II) oxide hydrate, palladium pivalate, palladium(II) propionate, palladium(II) sulfate, palladium(II) trifluoroacetate, PCy₃ Pd G2, Pd-PEPPSI™-IPent catalyst, [Pd(terpy)(2-Cl-phen)](BF4)2, [Pd(terpy)(MeCN)][BF₄]₂, PEPPSI™-IPr catalyst, PEPPSI™-SIPr catalyst, PPh₃ Pd G2, QPhos Pd G3, RockPhos Pd G3, RuPhos Pd G1 methyl t-butyl ether adduct, RuPhos Pd G2, RuPhos Pd G3, RuPhos Pd G4, salicylaldehyde thiosemicarbazone palladium(II) chloride, SPhos Pd G1, Methyl t-Butyl Ether Adduct, SPhos Pd G2, SPhos Pd G3, SPhos Pd G4, sSPhos Pd G2, fluBrettPhos Pd G3, tetraamminepalladium(II) acetate, tetraamminepalladium(II) bromide, tetraamminepalladium(II) chloride monohydrate, tetraamminepalladium(II) nitrate, tetrakis(acetonitrile)palladium(II) tetrafluoroborate, tetrakis(triphenylphosphine)palladium(0), [(1,3,5,7-tetramethyl-6-phenyl-2,4,6-trioxa-6-phosphaadamantane)-2-(2′-amino-1,1′-biphenyl)]palladium(II) methanesulfonate, (P)-Tol-BINAP Pd G3, (R)-TolBINAP Pd G4, P(o-tol)3 Pd G2, tris[μ-[(1,2-η:4,5-η)-(1E,4E)-1,5-bis(4-methoxyphenyl)-1,4-pentadien-3-one]]di-palladium, tris(dibenzylideneacetone)dipalladium(0), tris(dibenzylideneacetone)dipalladium(0), tris(dibenzylideneacetone)dipalladium(0):BINAP:Sodium tert-butoxide, tris(dibenzylideneacetone)dipalladium(0)-chloroform adduct, tris(dibenzylideneacetone)dipalladium/tri-tert-butyl phosphonium tetrafluoroborate mixture, tris(3,3′,3″-phosphinidynetris(benzenesulfonato)palladium(0) nonasodium salt nonahydrate, XantPhos Pd G3, XPhos Pd G1, XPhos Pd G2, XPhos Pd G3, and XPhos Pd G4.

In some embodiments, examples of copper catalysts include, without limitation, copper(I) acetate, copper(I) iodide, copper(I) bromide, copper(I) chloride, copper(I) bromide dimethyl sulfide complex, tetrakis(acetonitrile)copper(I) hexafluorophosphate, copper(I) oxide, tetrakis(acetonitrile)copper(I) tetrafluoroborate, copper(I) trifluoromethanesulfonate toluene complex, copper(I) trifluoromethanesulfonate benzene complex, copper(I) thiophene-2-carboxylate, copper(I) chloride-bis(lithium chloride) complex, iodo(triethyl phosphite)copper(I), copper (I) diphenylphosphinate, tetrakisacetonitrile copper(I) triflate, bromotris(triphenylphosphine)copper(I), (1,10-phenanthroline)(trifluoromethyl)copper(I), bis[(tetrabutylammonium iodide)copper(I) iodide], chloro(1,5-cyclooctadiene)copper(I) dimer, chloro[1,3-bis(2,6-diisopropylphenyl)imidazol-2-ylidene]copper(I), chloro[1,3-bis(2,4,6-trimethylphenyl)imidazol-2-ylidene]copper(I), bis(1,3-bis(2,6-diisopropylphenyl)imidazol-2-ylidene)copper(I) tetrafluoroborate, iodo[4,5-bis(diphenylphosphino)-9,9-dimethylxanthene]copper(I), and/or DABCO®-CuCl complex. In some embodiments, the copper catalyst is present in the reaction mixture in an amount of 0.01 to 0.5 M relative to the luminophore including a leaving group covalently bound thereto. Copper-catalyzed direct arylation of heterocycles with aryl bromides is described, for example, in Zhao et al., Angew. Chem. Int. Ed., 2009, 48, 3296-3300, incorporated herein by reference in its entirety.

In some embodiments, the reaction mixture further includes a base in an amount of 1M or more (e.g., 2M or more, 3M or more, 4M or more, 5M or more, 6M or more, 7M or more, 8M or more, or 9M or more) and/or 10M or less (e.g., 9M or less, 8M or less, 7M or less, 6M or less, 5M or less, 4M or less, 3M or less, or 2M or less) relative to the halogenated luminophore, such as K₃PO₄, Cs₂CO₃, K₂CO₃, Na₂CO₃, sodium acetate, potassium acetate, and/or cesium acetate. In some embodiments, the reaction mixture further includes a catalyst ligand in an amount of 0.01M or more (e.g., 0.1M or more, 0.3M or more, 0.5M or more, or 0.7M or more) and/or 1M or less (e.g., 0.7M or less, 0.5M or less, 0.3M or less, or 0.1M or less) relative to the luminophore including a leaving group covalently bound thereto, such as a phosphine ligand, Hacac, L-proline, 1,1′-bi-2-naphthol (BINOL), phenanthroline(Phen), and/or tetramethylethylenediamine (TMEDA). In some embodiments, the reaction mixture further includes an acid in an amount of 0.01M or more (e.g., 0.1M or more, 0.3M or more, 0.5M or more, or 0.7M or more) and/or 1M or less (e.g., 0.7M or less, 0.5M or less, 0.3M or less, or 0.1M or less) relative to the luminophore including a leaving group covalently bound thereto, such as pivalic acid, acetic acid, 1-adamantanecarboxylic acid, neodecanoic acid, benzoic acid, and/or nitro-benzoic acid. In certain embodiments, the reaction mixture further includes an organic solvent, such as N,N′-dimethylformamide (DMF), dimethylacetamide (DMAc), xylene, toluene, tetrahydrofuran, anisole, cyclopentyl methyl ether, 1,4-dioxane, 2-methyltetrahydrofuran, and/or methyl tert-butyl ether.

In certain embodiments, reacting the luminophore including a leaving group covalently bound thereto and the activated compound in a direct arylation reaction of the present disclosure is conducted at a temperature of 25° C. to 200° C. (e.g., 25° C. to 150° C., 50° C. to 200° C., 50° C. to 150° C., or 100° C. to 150° C.), under inert atmosphere (e.g., N₂ or argon), and/or at a pressure of 1 atm to 10 atm (e.g., 1 atm to 8 atm, 1 atm to 5 atm, 1 atm to 3 atm, or 1 atm).

The synthesis of the luminescent molecule, including the synthesis of the precursors (e.g., moiety including a heteroaryl core, and/or luminophore including a leaving group covalently bound thereto) can be free of organolithium compounds. In some embodiments, the reaction mixture does not include an organolithium compound.

Devices

The luminescent molecules of the present disclosure can be incorporated into a device. For example, the device can include a light emitting diode, a laser, a solar concentrator, green house coverage, and/or down-converting materials. In some embodiments, the luminescent molecules of the present disclosure can be used as a luminescent label for a biological sample. When used as a luminescent label for a biological sample, the luminescent molecule can be further functionalized with a reactive group suitable for bioconjugation, such as, for example, a N-hydroxysuccinimidyl (NHS)-ester, an isocyanate, an isothiocyanate, a benzoyl fluoride, a maleimide, an iodoacetamide, a 2-thiopyridine, a 3-arylpropiolonitrile, a carboxylate, a phosphonate, a sulfonate or any combination thereof. Functionalization of luminescent molecules and labeling of biological samples, are described, for example, in Bioconjugate Techniques, 3^{rd} Ed., by G. T. Hermanson, Academic Press; 3rd Edition (Sep. 2, 2013), herein incorporated by reference in its entirety.

The following examples are provided to illustrate, not limit, the disclosure.

Example 1 describes the synthesis and characterizations of xanthine moieties covalently bound to luminophores (e.g., pyrene). While xanthine moieties are described, it is understood that the synthetic methods, characterization methods, and implementation of the luminescent molecules into devices can be applied to other heteroaryl core-containing moieties and luminophores, as will be understood to a person of ordinary skill in the art. Example 2 describes the synthesis and characterizations of a variety of luminescent molecules incorporating xanthines, covering a range of optoelectronic properties.

### EXAMPLES

### Example 1. Synthesis of Pyrene Luminophores Covalently Bonded to Xanthine Derivatives

A green and scalable method to synthesize organic luminophores with minimal aggregation caused quenching is presented herein, where direct arylation is used to attach functionalized xanthine moieties onto luminophores. Using xanthine reduces manufacturing cost and minimizes safety risks associated with pyrophoric reagents otherwise required. Direct arylation provides atom and step economy, bypassing substrate pre-functionalization required for traditional Suzuki coupling. Moreover, xanthine can be easily functionalized via N-substitution, allowing tunable control over the processability and functionalities of the final products, and for optimization of performance for various applications. The efficacy of this method was demonstrated: after introducing xanthine moieties onto pyrene, an aggregation-quenched luminophore, resultant compounds demonstrated high solid-state photoluminescent quantum yields. The minimized aggregation quenching can be ascribed to the large dihedral angles that xanthine moieties introduced into these molecules, decreasing or preventing π-π interaction between luminophores. Furthermore, the large dihedral angles promoted the formation of hybridized local and charge-transfer state in these molecules, which is crucial to highly efficient organic light emitting diodes. Amplified spontaneous emission measurements were performed on these molecules demonstrate their use in organic lasers. Other molecules with minimized aggregation quenching can be synthesized with this method, coupling xanthine moieties with other luminophores via direct arylation.

### Characterization Instrumentation

Absorption spectra of chemicals were obtained using a Perkin Elmer Lambda 950-UV Vis/NIR spectrophotometer while the photoluminescence spectrums were obtained using a homemade instrument. PLQY data were obtained using an integrating sphere (Hamamatsu, C9920-12). Solution samples were prepared by dissolving PT1, PT2 and PT4 in to chloroform with concentration of 10⁻⁵ M. Thin film samples were prepared by spin-coating 10 mg/mL chloroform solutions of PT1, PT2 and PT4 respectively onto glass substrates in rate of 1000 rpm.

Nuclear magnetic resonance (NMR) spectra were obtained with a Bruker AV500 at room temperature. Samples are dissolved in CHCl₃-d in 10 mg/mL.

Single crystal of PT1 and PT4 were prepared by vapor diffusion. PT1: an open vial of 5 mg/mL CF solution was placed into acetone atmosphere and left in the absence of turbulence for 2 days. PT4: an open vial of 5 mg/mL CF solution was placed into EtOH atmosphere and left in the absence of turbulence for 2 days. Single crystal of PT2 was prepared by slow crystallization. PT2 was heating in toluene to 100° C. in a large water bath and toluene was added into the solution till it is fully dissolved. The system was then left undisturbed for one day.

### Material Synthesis

Theobromine, 1-bromooctane, 1-bromopyrene, 1,6-dibromopyrene, 1,3,6,8-tetrabromopyrene and tris(2-methoxyphenyl)phosphine were purchase from TCI. Bis(dibenzylideneacetone)palladium(0) and pivalic acid were purchased from Sigma. Solvents used were purified via a DriSolv® solvent purification system from Inert Inc. K₂CO₃ and Cs₂CO₃ were ground into a powder and dried at 120° C. overnight before reactions. Reactions were run under N₂ atmosphere using standard Schlenk techniques and detailed synthesis procedures are described below.

3,7-dimethyl-1-octyl-3,7-dihydro-1H-purine-2,6-dione (Theo8). Into a 500 mL round bottom flask, theobromine (18.0 g, 100 mmol), 1-bromooctane (23.2 g, 120 mmol), dried K₂CO₃ (20.7 g, 150 mmol) and 200 mL DMF was added. The system was degassed and then heated to 120° C. overnight. When finished, the reaction was cooled to room temperature. ethyl acetate was used to extract the product and brine was used to remove residue DMF. The organic layer was subsequently washed with brine to remove the DMF in the organic phase. The organic layer was then dried over Mg₂SO₄ and concentrated under reduced pressure. The crude product was further purified with column chromatography using dichloromethane/methanol in a 10:1 ratio as an eluent. The collected fraction was then recrystallized from hexane. 26.5 g of collected white solid was collected, with 88% yield. ¹H NMR (500 MHz, CDCl₃) δ 7.51 (s, 1H), 4.02-3.97 (m, 5H), 3.57 (s, 3H), 1.70-1.58 (m, 2H), 1.47-1.20 (m, 10H), 0.92-0.82 (m, 3H). ¹³C NMR (126 MHz, CDCl₃) δ 155.17, 151.41, 148.73, 141.61, 107.59, 41.39, 33.48, 31.84, 29.56, 29.34, 29.24, 28.12, 27.05, 22.64, 14.07.

3,7-dimethyl-1-octyl-8-(pyren-1-yl)-3,7-dihydro-1H-purine-2,6-dione (PT1). Theo8 (552 mg, 2 mmol) and 1-bromopyrene (562 mg, 2 mmol), pivalic acid (40 mg, 0.4 mmol) and dried Cs₂CO₃ (1.30 g, 4 mmol) were added into a 25 mL round bottom flask. 10 ml toluene was then adding into the system followed by degassing with N₂ flow for 10 min. Tris(2-methoxyphenyl)phosphine (56 mg, 0.097 mmol) and bis(dibenzylideneacetone)palladium(0) (40 mg, 0.044 mmol) were added to the solution under N₂ flow, and the solution turned purple. The flask was then sealed with a stopcock and heated to 100° C. After reacting for 1 day, the system was cooled and filtered. The organic phase was then concentrated under reduced pressure. The crude product was further purified with column chromatography using chloroform/methanol in a 400:5 ratio as an eluent (chloroform/methanol=400/5). 715 mg of white solid was obtained in a 75% yield. ¹H NMR (500 MHz, CDCl₃) δ 8.29 (d, J=2.5 Hz, 1H), 8.28 (d, J=2.5 Hz, 1H), 8.26 (d, J=10 Hz, 1H), 8.20 (d, J=9.0 Hz, 1H), 8.15 (q, 2H), 8.08 (t, 2H), 7.92 (d, J 9.2 Hz, 1H), 4.19-4.02 (m, 2H), 3.83 (s, 3H), 3.70 (s, 3H), 1.83-1.69 (m, 2H), 1.50-1.23 (m, 10H), 0.98-0.83 (m, 3H). ¹³C NMR (126 MHz, CDCl₃) δ 155.58, 152.03, 151.65, 150.16, 148.59, 132.90, 131.26, 130.74, 130.40, 129.40, 129.14, 127.80, 127.19, 126.61, 126.30, 126.08, 124.84, 124.60, 124.42, 123.90, 122.40, 108.58, 41.67, 33.49, 31.88, 29.88, 29.29, 28.27, 27.14, 22.67, 14.08.

8,8′-(pyrene-1,6-diyl)bis(3,7-dimethyl-1-octyl-3,7-dihydro-1H-purine-2,6-dione) (PT2). Theo8 (1,932 mg, 7 mmol) and 1,6-dibromopyrene (1,080 mg, 3 mmol), pivalic acid (80 mg, 0.8 mmol) and dried Cs₂CO₃ (3 g, 9 mmol) were added into a 50 mL round bottom flask. 25 mL toluene was then adding into the flask, followed by degassing with N₂ flow for 10 min. Tris(2-methoxyphenyl)phosphine (122 mg, 0.194 mmol) and bis(dibenzylideneacetone)palladium(0) (80 mg, 0.088 mmol) were added to the solution under N₂ flow, and the solution turned purple. The flask was then sealed with a stopcock and heated to 100° C. After reacting for 1 day, the system was cooled and filtered. The organic phase was then concentrated under reduced pressure. The crude product was further purified with column chromatography using chloroform/methanol in a ratio of 400/5 as an eluent. 1,472 mg pale yellow solid was obtained in a 65% yield. ¹H NMR (500 MHz, CDCl₃) δ 8.38 (d, J=7.9 Hz, 2H), 8.23 (d, J=9.2 Hz, 2H), 8.16 (d, J=7.9 Hz, 2H), 8.09 (d, J=9.2 Hz, 2H), 4.16-4.05 (m, 4H), 3.88 (s, 6H), 3.71 (s, 6H), 1.80-1.69 (m, 2H), 1.51-1.22 (m, 20H), 0.94-0.85 (m, 6H). ¹³C NMR (126 MHz, CDCl₃) δ 155.54, 151.55, 151.38, 148.51, 132.31, 130.48, 129.11, 128.51, 125.64, 124.54, 123.65, 108.66, 41.68, 33.59, 31.86, 29.89, 29.39, 29.27, 28.23, 27.11, 22.66, 14.11.

8,8′,8″,8′″-(pyrene-1,3,6,8-tetrayl)tetrakis(3,7-dimethyl-1-octyl-3,7-dihydro-1H-purine-2,6-dione) (PT4). Theo8 (1,932 mg, 76 mmol) and 1,3,6,8-tetrabromopyrene (517 mg, 1 mmol), pivalic acid (80 mg, 0.8 mmol) and dried Cs₂CO₃ (3 g, 9 mmol) were added into a 50 mL round bottom flask. 15 mL toluene was then adding into the system followed by degassing with N₂ flow for 10 min. Tris(2-methoxyphenyl)phosphine (122 mg, 0.194 mmol) and bis(dibenzylideneacetone)palladium(0) (80 mg, 0.088 mmol) were added to the solution under N₂ flow, and the solution turned purple. The flask was then sealed with a stopcock and heated to 100° C. After reacting for 1 day, the system was cooled and filtered. The organic phase was then concentrated under reduced pressure. The crude product was purified with column chromatography using chloroform/methanol in a ratio of 40:1 as an eluent. 585 mg green-yellow solid was obtained in a 43% yield. ¹H NMR (500 MHz, CDCl₃) δ 8.36 (s, 2H), 8.27 (s, 4H), 4.08 (t, 8H), 3.91 (s, 12H), 3.68 (s, 12H), 1.73 (m, 8H), 1.47-1.23 (m, 40H), 0.92-0.87 (m, 12H). ¹³C NMR (126 MHz, CDCl₃) δ 155.44, 151.42, 149.53, 148.51, 131.43, 131.14, 127.32, 124.79, 124.64, 109.01, 41.76, 33.86, 31.84, 29.89, 29.37, 29.26, 28.18, 27.08, 22.66, 14.10.

### Material Synthesis and Thin Film PLQY

The synthesis route of xanthine-pyrene complexes is shown in FIG. 3D. Referring to FIG. 3D, alkylation of theobromine proceeds in a simple and high yield reaction to obtain Theo8. The Theo8 molecule was attached to pyrene via C—H activation, converting mono-, di- and tetra-bromopyrene into PT1, PT2 and PT4 respectively. The structures of all three compounds were confirmed by ¹H-NMR and ¹³C-NMR, as detailed above. These three compounds show good solubility in common organic solvents, facilitated by the alkylation of the xanthine.

### Photophysical Properties

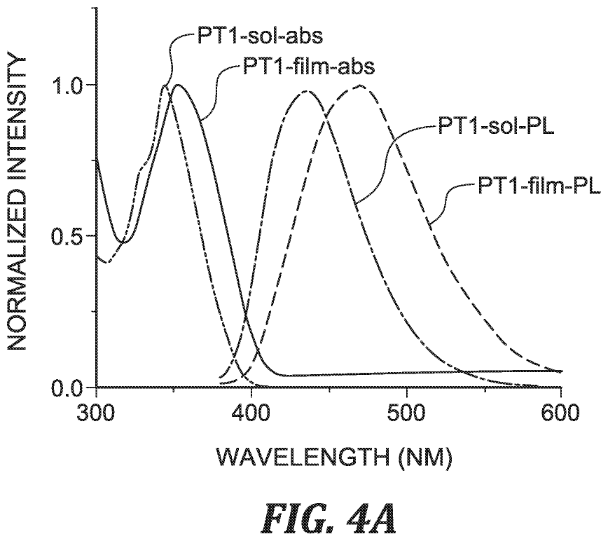

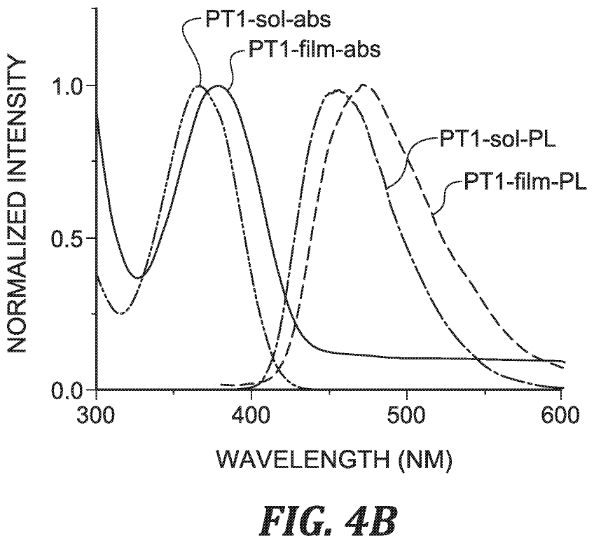

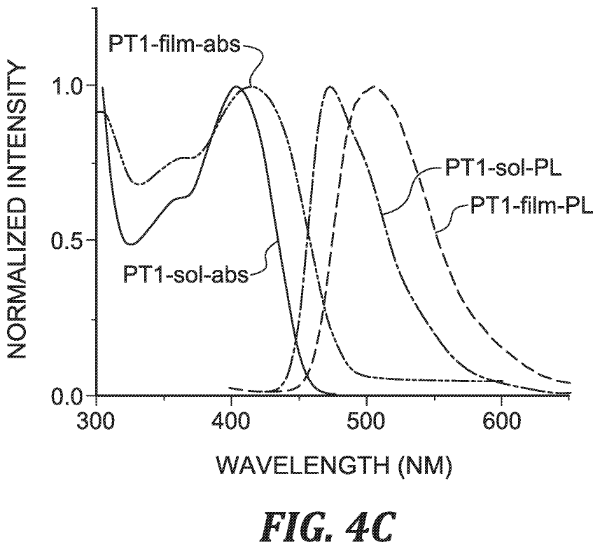

FIGS. 4A-4C show the UV-vis absorption and photoluminescence (PL) spectrum of PT1, PT2 and PT4 in 10⁻⁵ M chloroform solution and as thin film on glass substrate. Spectra details are summarized in Table 1. All three molecules exhibited large Stokes shift as thin film, 115 nm for PT1 105 nm for PT2 and 100 nm for PT4, which rendered them great candidate as laser gain medium with low parasitic loss from ground state re-absorption. FIG. 5 shows the position of their highest occupied molecular orbitals (HOMOs) and lowest unoccupied molecular orbitals (LUMOs), which are suitable for charge injection in OLED devices.

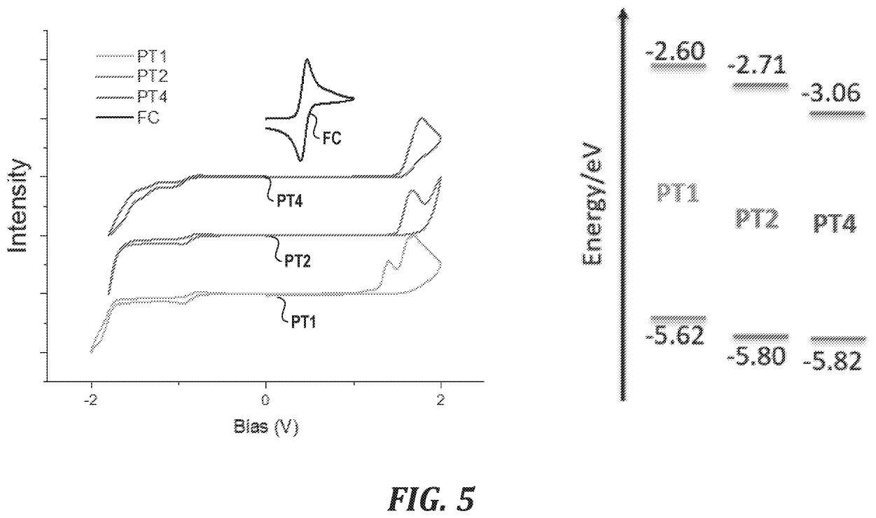

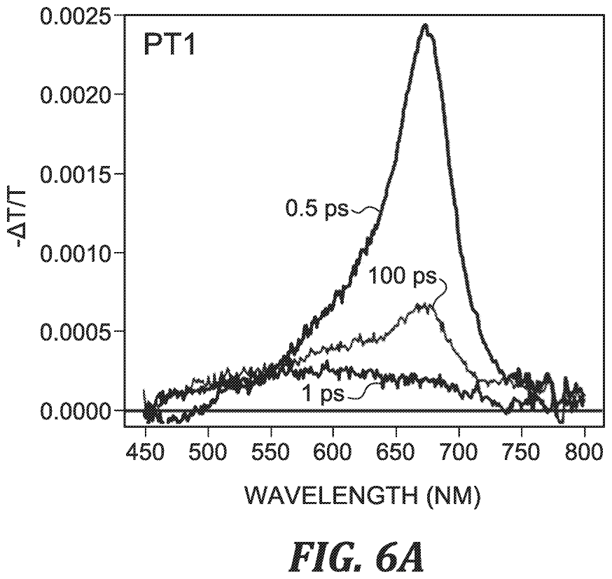

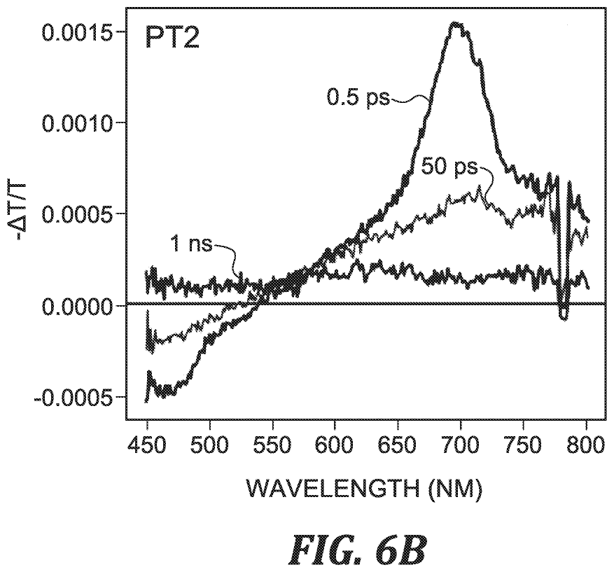

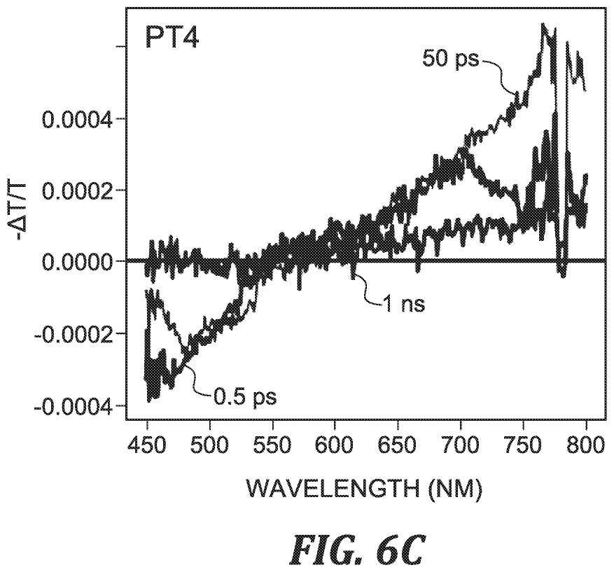

Transient absorption was utilized to analyze the parasitic loss of the gain medium from the excited-state absorption. As can be seen from FIGS. 6A-6C, three molecules had strong excited state absorption above 550 nm, which rapidly decayed after 1 ns. These excited state absorptions had little overlap with their corresponding ground state PL emissions (negative signals under 550 nm). Therefore, these three molecules hold great potential in laser applications as they should have low parasitic loss in the gain medium.

TABLE 1
Summary of UV-vis and PL spectra details of PT1, PT2 and PT4.

| | Solution | | | | | Thin Film | | | | |
|---|---|---|---|---|---|---|---|---|---|---|
| | abs, | PL, | abs, | Energy | Stokes | abs, | PL, | abs, | Eg/ | Stokes |
| | max/nm | max/nm | onset/nm | gap/eV | shift/nm | max/nm | max/nm | onset/nm | eV | shift/nm |
| PT1 | 343 | 432 | 395 | 3.13 | 89 | 360 | 475 | 409 | 3.03 | 115 |
| PT2 | 367 | 455 | 420 | 2.95 | 89 | 380 | 485 | 440 | 2.81 | 105 |
| PT4 | 400 | 474 | 457 | 2.71 | 74 | 430 | 530 | 490 | 2.53 | 100 |

The PLQYs of these thin film samples were high: PT1 thin film had a PLQY as high as 95%, while the PLQY of PT2 was 90%, and that of PT4 was 74%. The PLQY of pyrene in diluted solution was 100% while it is completely quenched in solid. These high solid-state fluorescence implied that the ACQ of chromophore was suppressed effectively. Without wishing to be bound by theory, it is believed that alkylated xanthine acted as a spacer for chromophores to decrease or prevent the parasitic π-stacking interactions between adjacent chromophores, and thus increased the solid state PLQY. With more xanthine content in a molecule (i.e., going from PT1 to PT4), the PLQY decreased moderately.

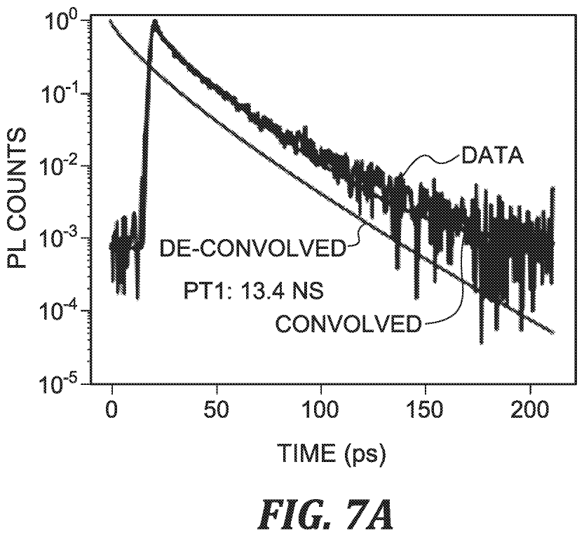

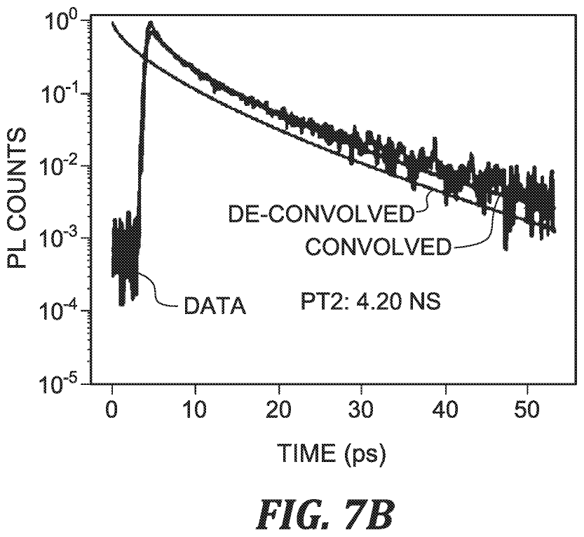

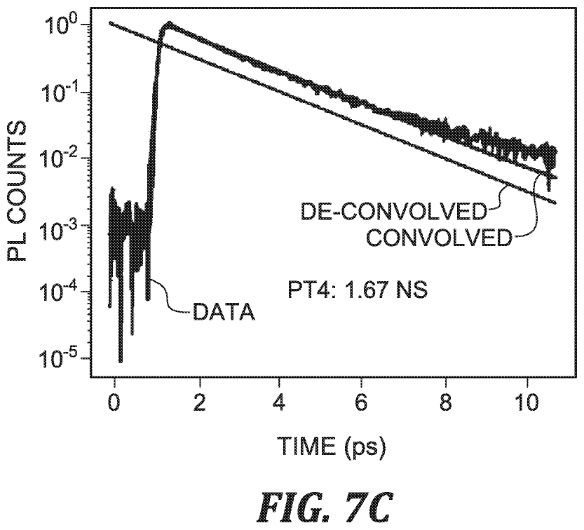

Without wishing to be bound by theory, it is believed that the increased polarity of the molecules facilitates non-radiative decay. The increase polarity of the molecules facilitates the formation of charge transfer state, which is weakly fluorescent. As the xanthine ratio in the complex increases, the content of polar lactam moieties increases as well. It is believed that this leads to a more favored formation of charge transfer states, accelerating the non-radiative decay of excited states, which can explain the strong linear dependence of the nonradiative decay rate on the number of attached caffeine attached (FIGS. 7A-7C).

### Crystal Structures

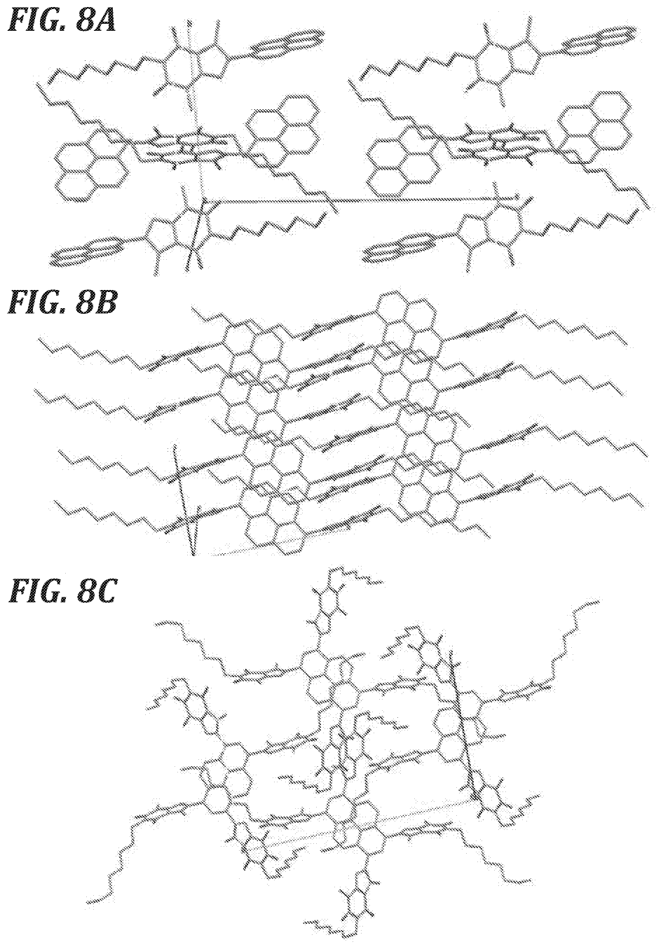

To explain the high solid state PLQY for these molecules, single crystal X-ray diffraction (SCXRD) was used to study the molecular packing of PT1, PT2 and PT4. In these molecules, the dihedral angles between xanthine moieties and pyrene were large, at 63.12°, 80.66° and 47.44° for PT1, PT2 and PT4 respectively. These large dihedral angles originated from the steric repulsion between pyrene and the adjacent methyl group on xanthine moieties, which forced these molecules to adopt a less planar conformation. FIGS. 8A-8C show the molecular arrangement of PT1, PT2 and PT4 in crystals, demonstrating that the chromophores of all three molecules were well isolated and prevented from aggregation. It is believed that these two factors explain the high PLQYs of these molecules in the solid state.

PT1 and PT2 showed similar packing, where the xanthine moieties self-assemble onto one another driven by strong polar interaction between lactam groups. Meanwhile, pyrenes and alkyl chains were arranged accordingly, driven by the positioning of the xanthine moieties, and adopted alternating arrangement alongside the xanthine domains. As a result, adjacent pyrenes were offset from each other and ACQ between pyrenes was suppressed in the solid state. PT4 adopted a different packing pattern. Yet pyrenes were still well separated from one another, and thus PT4 was also fluorescent in solid state. The crystal structures of all three molecules demonstrate that xanthine moieties serve as spacers to isolate chromophores in molecules.

### Density Function Theory (DFT) Simulations

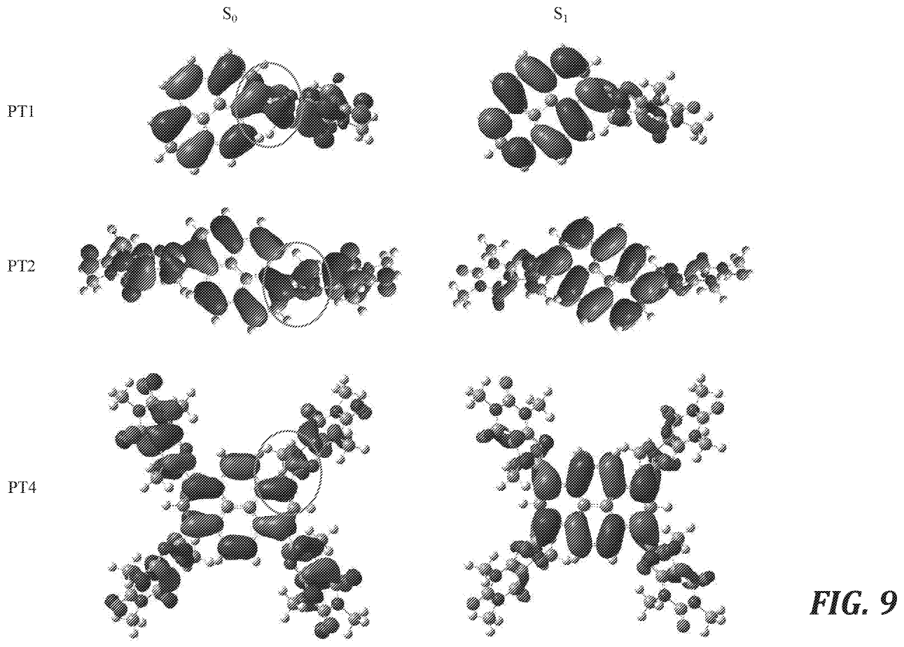

As shown in FIGS. 4A-4C, when the xanthine content was increase from PT1 to PT4, a redshift of both absorption and emission spectra was observed. With more xanthine attached to the pyrene core, the size of the conjugated system increased. This implied that xanthine was incorporated as apart of the conjugated system. Density function theory (DFT) simulations of molecular orbitals (MOs) were performed to look into how conjugations were formed between xanthine and pyrene despite the large dihedral angles. As shown in FIG. 9, there was an unusual distribution HOMOs of both PT1 and PT2. Despite the large dihedral angle offset of the pyrene and xanthine moieties, 63.12° for PT1 and 80.66° for PT2, orbital symmetries allowed for orbital overlap between pyrene and xanthine, intertwining around the single bond between the pyrene and xanthine. For PT4, having a relatively smaller dihedral angle of 47.44°, orbitals were able to extend through the molecule.

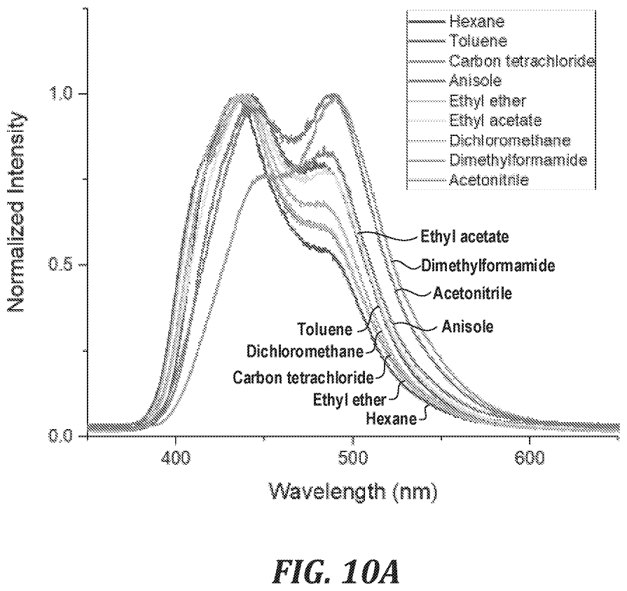

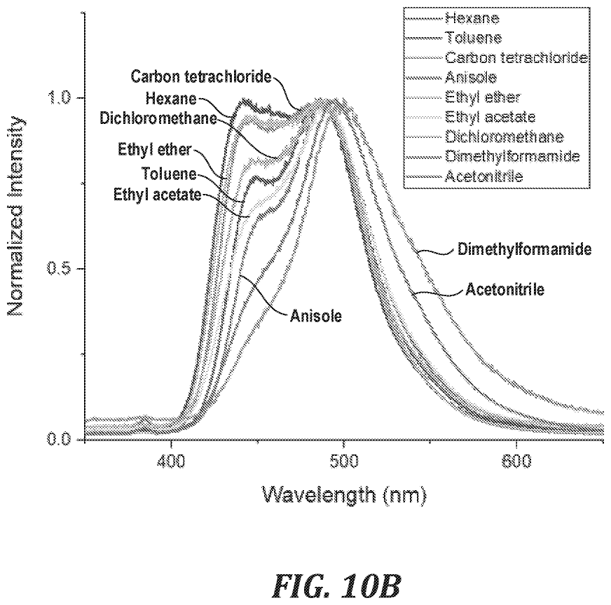

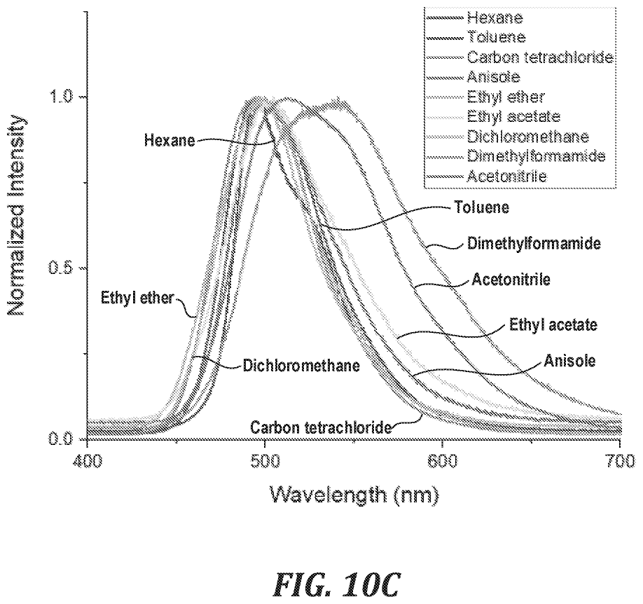

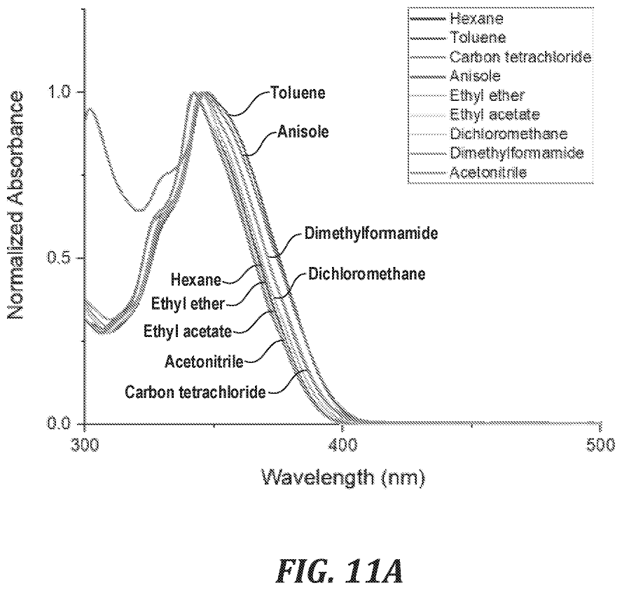

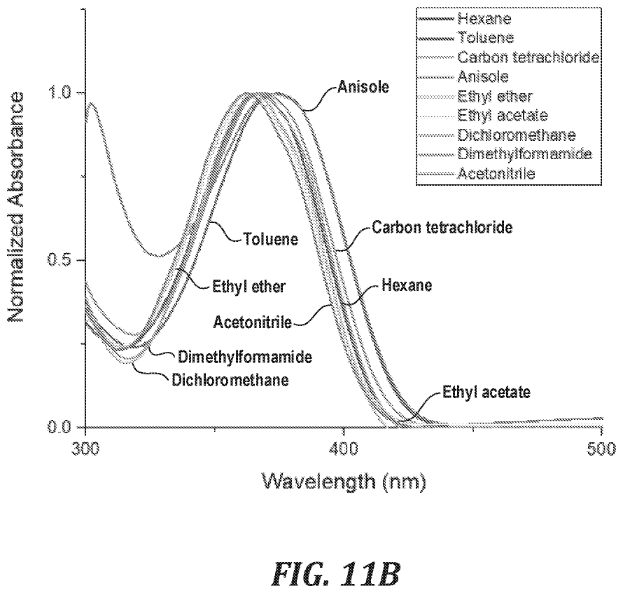

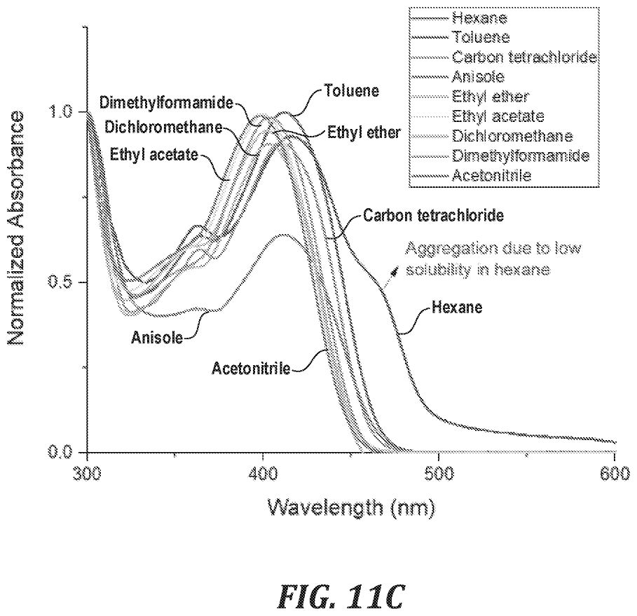

Moreover, in all three molecules MOs were spread out among the whole molecules in HOMOs, while their LUMOs were more localized onto pyrene moieties. Thus the HOMO→LUMO transition contained a major part of locally excited (LE) transition of pyrene and a minor part of intra-molecular charge transfer (ICT) transition from xanthine to pyrene, implying the LUMO was a hybridized local and charge-transfer excited (HLCT) state, as a result of LE state and ICT state inter-crossing. Follow-up fluorescent solvatochromic experiments further confirmed the existence of HLCT states in PT1, PT2 and PT4. As shown in FIGS. 10A-10C, there were two major peaks in the PL spectra of each molecules: the one of higher energy (or shorter wavelength) corresponded to the radiative decay from LE state, while the other one corresponded to ICT state. As the polarity of the solvents was increase, the progression where the PL peak of LE state diminished while the peak of ICT state intensified was observed. Due to the polar nature of ICT states, they were stabilized by the polar environment and thus their energy levels downshift as solvent polarity increased. On the other hand, the energy level of LE state changed little with solvent polarity. Because PT1, PT2 and PT4 possessed LE states and ICT states that were energetically adjacent, the crossover of their contributions to the HLCT state (in this case from LE state dominant to ICT state dominant) could be tuned by increasing the solvent polarity to downshifting the ICT state energy. The absorption and emission solvatochromic experiments of a) PT1, b) PT2 and c) PT4 at a concentration of 10⁻⁵ M at room temperature was shown in FIGS. 10A-10C and 11A-11C.

HLCT states are crucial for the design of next-generation electroluminescence devices, where they convert triplet excitons into singlets excitons for fluorescence, breaking the theoretical limit of fluorescence devices. Large dihedral angles can be responsible for the formation of HLCT states. Using the method of the present Example, attaching xanthine moieties onto luminophores via direct arylation, large dihedral angles could be easily introduced into organic luminophores, inducing HLCT states in resulted materials.

### Temperature Dependent PL Measurement

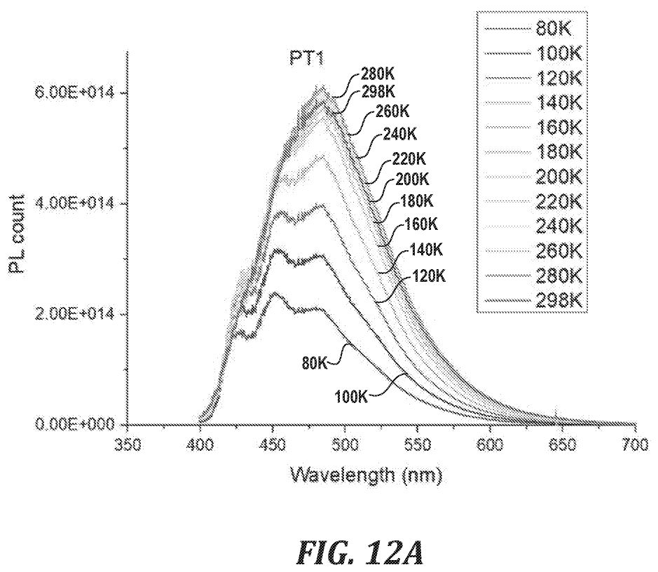

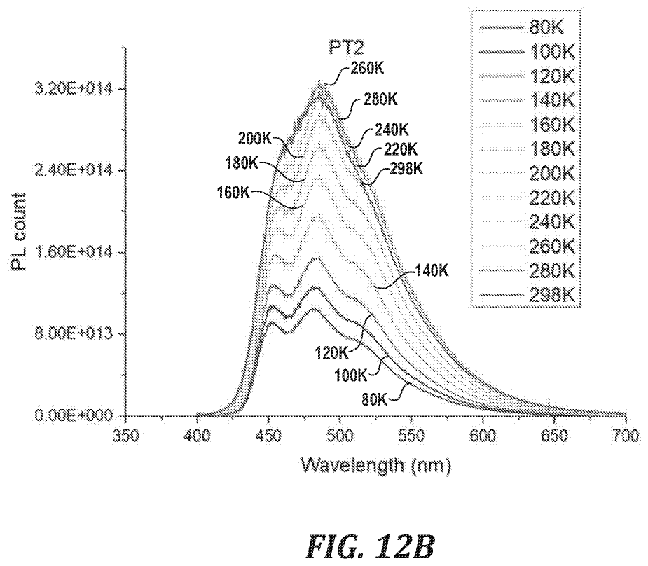

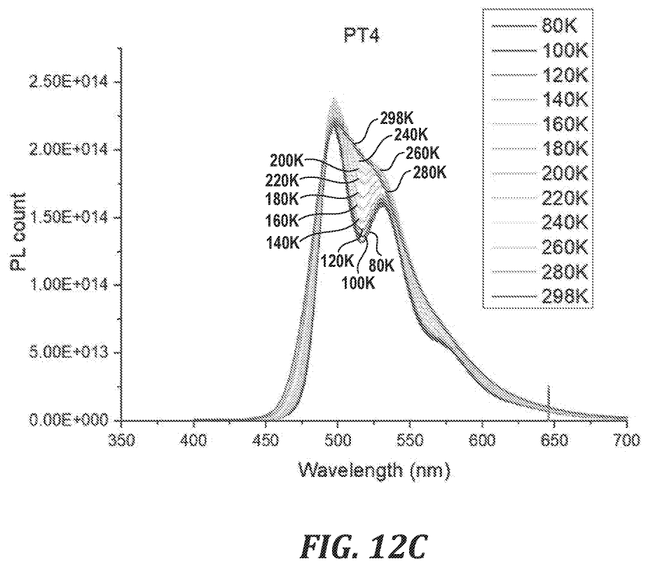

Temperature dependent PL measurement were conducted to find the optimal operation temperature for laser application, see FIGS. 12A-12C. At low temperatures, vibronic progressions were observed for all molecules, with vibronic spacing around 0.15 eV, indicating the coupling between electronic transitions and aromatic C—H bond stretching and bending. As the temperature increased, molecular movements became more active and frequent, which led to a large range of molecular conformations with small energy variations. Thus, vibronic features became less sharp as the temperature increased.

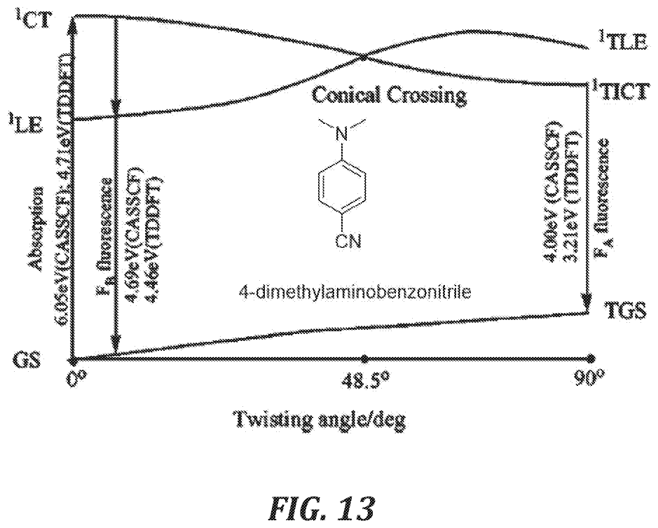

All three molecules had negative thermal quenching effect—PL intensity increased with temperature increases. This is not commonly observed in either organic or inorganic materials. Without wishing to be bound by theory, it is believed that this effect originated from the large dihedral angle between pyrene and caffeine. For example, a model of the energy level of 4-dimethylaminobenzonitrile as a function of the dihedral angle between the dimethylamino group and benzene ring is shown in FIG. 13 (see, e.g., X. Xu, Z. Cao, Q. Zhang, J. Chem. Phys., 2005, 122, 194305). As the dihedral angle increased, the energy of LE state increased while CT state decreased. It is believed that this was because at large dihedral angle MOs were not sufficiently conjugated, and it would be difficult to extend MO across the whole molecule to adopt LE state. On the other hand, CT state was more favored because of this large dihedral angle. At a certain angle, 48.5° for 4-dimethylaminobenzonitrile, the energy curves of CT and LE crossed over. Below 48.5° LE had a lower energy while beyond 48.5° CT has a lower energy.

Following this trend between energy levels and dihedral angle, it is believed that their CT states are lower than LE states, considering all three molecules adopted large dihedral angles. Thus, at low temperature, excited population was trapped in non-radiative CT states because of their lower energy. As temperature increased, more and more population excited from the CT state to the highly emissive LE state by thermal energy, kT. Additionally, the negative thermal quenching effects were more pronounced in the molecules with a large dihedral angle, which had a lower CT state position compared to its LE state. As shown in Table 2, PT2 tripled its PL intensity, which was the integration of the PL curve, from 80 K to 298 K. This was because PT2 has the largest dihedral angle of three, and thus it had the largest offset between LE state and CT state. Therefore, in PT2, a largest number of excited population of all three molecules tended to stay in the non-radiative CT state at low temperature because it was more thermally favored. However, when temperature increased this largest population in CT state would be reactivated onto LE state and thus PT2 had the largest increase in the fluorescent. The dihedral angle for PT1 is 63.12°, which was smaller than PT2, and thus its PL intensity showed a 2.5-fold increase. However, PT4 has only 1.3 times PL increase when the temperature was raised from 80 K to 298 K. This was because it had the smallest dihedral angle of all, 47.44°, and this number was believed to be close to its own conical crossing point of LE and CT. In fact, the energy difference of CT and LE of PT4 is so small that even at 80 K, the population in the CT state could be converted to LE state by thermal energy. PT4 has the least negative thermal quenching effect. All three molecules showed the highest PL intensity around 298 K. This high performance at room temperature made these materials exceptionally promising for lasing gain media, as they do not require active cooling for maximal performance.

TABLE 2
PL count integration of each curve in FIGS. 12A-12C in varied temperature.

| PL count | | | | | | | | | | | | |
|---|---|---|---|---|---|---|---|---|---|---|---|---|
| integration/10¹⁶ | 80K | 100K | 120K | 140K | 160K | 180K | 200K | 220K | 240K | 260K | 280K | 298K |
| PT1 | 6.32 | 8.92 | 11.35 | 13.67 | 15.51 | 15.95 | 16.21 | 16.51 | 16.57 | 16.89 | 17.14 | 16.52 |
| PT2 | 2.61 | 3.17 | 3.92 | 5.02 | 6.07 | 6.95 | 7.81 | 8.36 | 8.51 | 8.78 | 8.70 | 8.46 |
| PT4 | 3.96 | 4.03 | 4.13 | 4.25 | 4.44 | 4.62 | 4.83 | 4.95 | 5.00 | 5.11 | 5.07 | 5.11 |

### Amplified Spontaneous Emission (ASE) Measurement

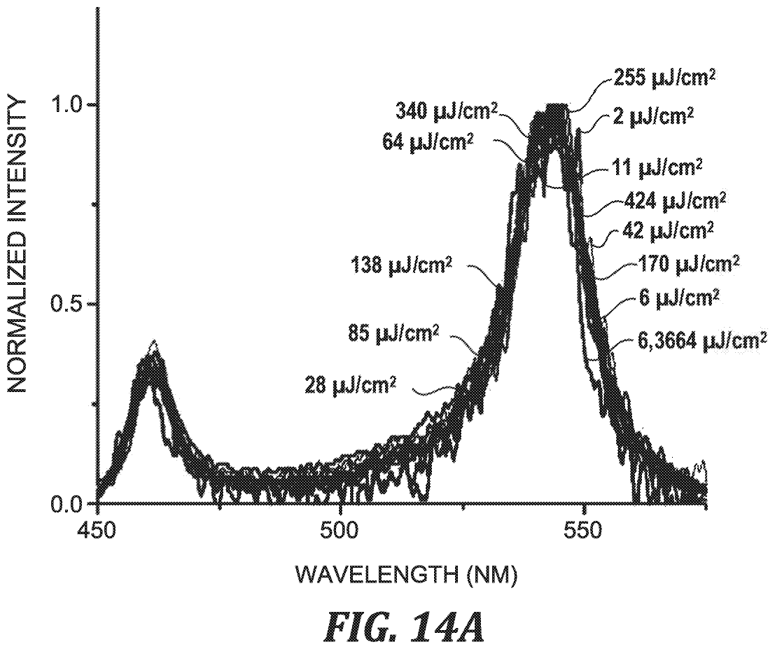

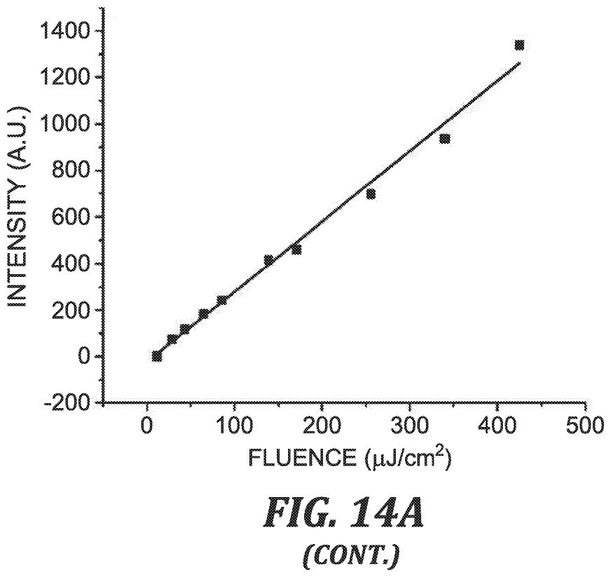

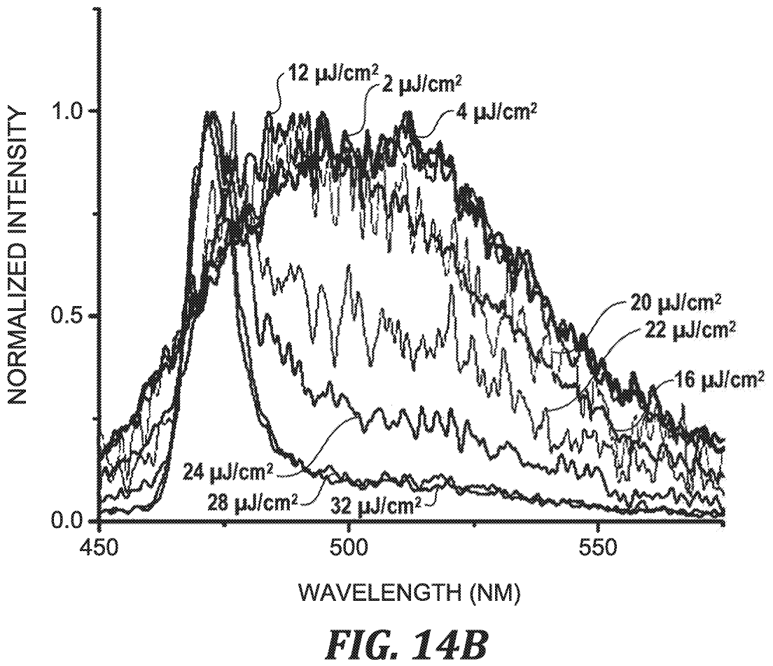

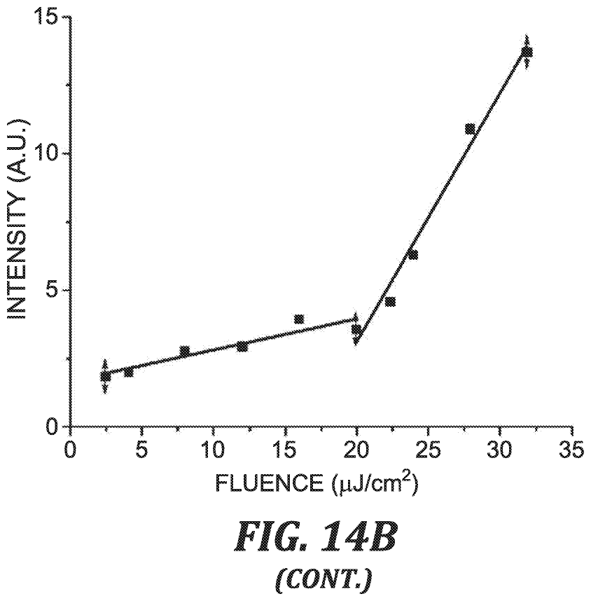

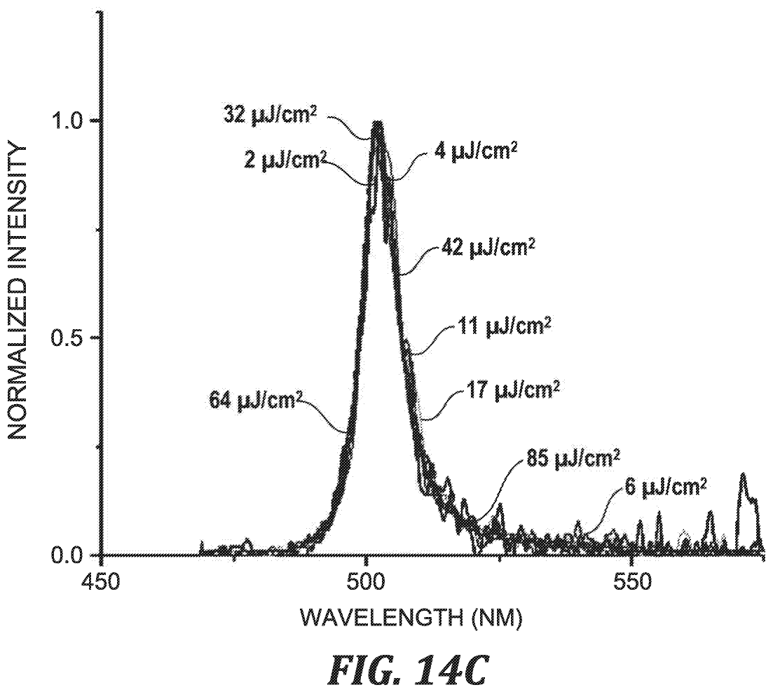

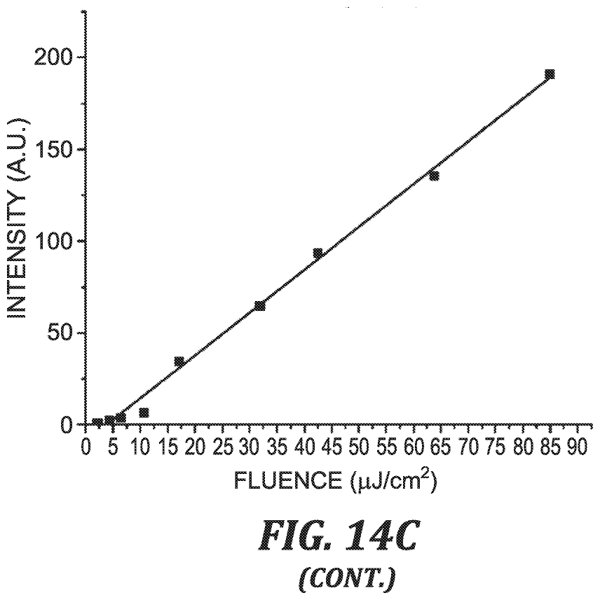

High solid-state PLQYs and large stoke shifts, shown in FIGS. 4A-4C, made these xanthine-pyrene complexes potential candidates for OL application. Considering amplified spontaneous emission (ASE) measurement is facile and appropriate techniques to compare the lasing performance of materials excluding effects of resonant cavity, ASE measurements of PT1, PT2, and PT4 thin films were performed (FIGS. 14A-14C). FIG. 14B (upper) is the ASE spectrum of a 90 nm PT2 thin film on a glass substrate. As excitation energy increased, a narrowing of the edge output spectrum was observed. In the FIG. 14B (lower), a change in slope of the output intensity took place as excitation fluence reached 20 μJ/cm2, and meanwhile the edge emission was abruptly narrowed, as shown in FIG. 14B (upper). This indicated that the ASE threshold for PT2 was 20 μJ/cm². ASE was not observed in PT1 and PT4 films of less than 50 nm, although waveguiding modes were observed in edge emission, where PL emission intensity did not show non-linearity over several orders of magnitude of pump intensity. A thick enough smooth film from PT1 and PT4 was not obtained.

Thus, in the present Example, ACQ in organic luminophores was effectively suppressed by modifying them with xanthine moieties via direct arylation. With this green and low-cost method, three highly fluorescent xanthine-pyrene prototypes were synthesized in two steps. SCXRD results showed the intermolecular t-t interaction between pyrenes were hindered due to the large dihedral angles introduced by xanthine moieties. As a result, ACQ was suppressed in these compounds and they were highly emissive both in solution and in solid form. In addition, large dihedral angles further induced the formation of HLCT states in these molecules, which was verified by DFT simulation and solvatochromic experiments. Their use in OL was investigated via ASE measurements, where PT2 showed an optimal threshold of 20 μJ/cm2. Thus, Example 1 presents a green and low-cost method to suppress ACQ in organic semiconductors and induces HLCT states at the same time, offering a solution for the scalable material production for electroluminescence devices.

### Example 2. Synthesis of Luminescent Molecules Incorporating Xanthines Having Enhanced Stability

A method to simultaneously increase of external quantum efficiency (EQE), processability, and stability of organic dyes is presented herein, where direct arylation is used to attach functionalized xanthine moieties onto luminophores. In the design of organic semiconductors, side chains are often introduced into their structures to enable/enhance their solution processability and thus facilitate fast and low-cost device fabrication. However, the presence of the side chains in organic semiconductors are also shown to accelerate photodegradation of the materials. In this section, a method to enhance the solution processability of organic dyes with minimized stability reduction is presented, by utilizing alkylated theobromines instead of only alkyl chains. The present Example showed that the introduction of alkylated theobromine lowered the energy levels of organic dyes and suppressed the formation of superoxide radicals, thereby slowing a major pathway of photodegradation. In addition, the resultant theobromine dyes formed highly fluorescent complexes with an industrial polymer poly (styrene-butadiene-styrene) (SBS), which possessed external quantum efficiencies (EQEs) around 90%, which were significantly higher than most inorganic phosphors, demonstrating high potential utility as green and red phosphors in phosphor converted light emitting diodes (pc-LEDs).

### Characterization and Instrumentation

Theobromine, 1-bromooctane, R305, DCJTB, 4,7-dibromo-benzo[c][1,2,5]thiadiazole were purchased from TCI. Bis(dibenzylideneacetone)palladium(0) anhydrous xylene and pivalic acid were purchased from Sigma Aldrich. K₂CO₃ and Cs₂CO₃ were ground into a powder and dried at 120° C. overnight before reactions. Reactions were run under N₂ atmosphere using standard Schlenk techniques and detailed synthesis procedures are described below. NMR was taken with Bruker 500 MHz spectrometer.

Ultraviolet photoelectron spectroscopy (UPS) sample preparation: ITO substrates were sonicated in acetone and iso-propanol respectively for 15 mins and then dried by air. They were later cleaned with ozone for 15 min. At ambient conditions (e.g., under air at room temperature of about 21° C.), organic dye chloroform solutions (30 mg/ml) were dynamically spin-coated on the clean ITO substrates at 2000 rpm for 30 s. The obtained samples were then dried overnight under high vacuum. UPS spectra were obtained with Kratos AXIS Ultra DLD with He (I) source and pass energy of 5 eV.

Absorption, PL, and PLQY sample preparation: Under ambient conditions, organic dye in chloroform solutions (20 mg/ml) were dynamically spin-coated onto glass substrates at 1000 rpm for 60 s. The obtained samples were then dried overnight under high vacuum of 10⁻⁷ mbar. Absorption spectra were taken with Perkin Elmer Lambda 950-UV Vis/NIR spectrophotometer; PL were measure with Ocean Optics FLAME spectrometer; PLQY data were obtained using an integrating sphere (Hamamatsu, C9920-12).

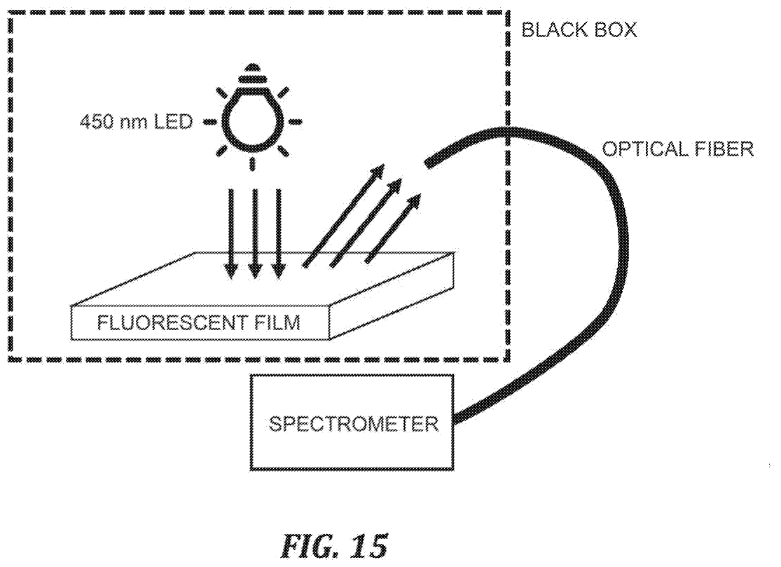

Stability test were performed with the setup shown in FIG. 15. Samples were prepared as follows:

1. Thin film samples: under ambient conditions, organic dye chloroform solutions (20 mg/ml) were dynamically spin-coated on glass substrate at 1000 rpm for 60 s. The obtained samples were then dried overnight under high vacuum of 10⁻⁷ mbar.

2. SBS complex: at ambient air, organic dye and SBS were dissolved in toluene (dye:SBS:toluene=1 mg:100 mg:1 ml). After the SBS was fully dissolved, 300 μl of the clear solution was drop-casted onto a 1.8×1.8 cm² glass substrate and dried under air flow. The obtained samples were then dried overnight under high vacuum of 10⁻⁷ mbar. (SBS were purchased from Sigma Aldrich, styrene 30 wt. %, average Mw ˜140,000 by GPC, contains <0.5 wt. % antioxidant)

Singlet oxygen generation monitoring: an organic dye (0.005 mmol) and a singlet oxygen scavenger (diphenyl anthracene) (0.005 mmol) were weighed and added to a beaker, 50 ml anisole was then added to the beaker, and a clear solution was obtained. The dye concentration and scavenger concentration were both 10⁻⁴ M. The same procedure was repeated for the other organic dyes. The resulted solutions in beakers were radiative in open air when irradiated with a 450 nm LED of ˜90 mW/cm², and solution samples were collected from beaker at 0, 20, 40, 60, 80 and 100 min. The UV-Vis spectra of these collected samples were measured, with 10⁻⁴ M anisole solution of the respective dyes serving as a reference, so that the changes in the diphenyl anthracene absorption could be monitored.

### Material Synthesis

8,8′-(benzo[c][1,2,5]thiadiazole-4,7-diyl)bis(3,7-dimethyl-1-octyl-3,4,5,7-tetrahydro-1H-purine-2,6-dione) (BT2). Theo8 (1,932 mg, 7 mmol) and 4,7-dibromobenzo[c][1,2,5]thiadiazole (873 mg, 3 mmol), pivalic acid (80 mg, 0.8 mmol) and dried Cs₂CO₃ (3 g, 9 mmol) were added to a 50 mL round bottom flask. 25 mL xylene was then added to the flask, followed by degassing with N₂ flow for 10 min. Tris(2-methoxyphenyl)phosphine (122 mg, 0.194 mmol) and bis(dibenzylideneacetone)palladium(0) (80 mg, 0.088 mmol) were added to the solution under N₂ flow, and the solution turned purple. The flask was then sealed with a rubber stopper and heated to 100° C. After reacting for 1 day, the system was cooled and filtered. The organic phase was then concentrated under reduced pressure. The crude product was further purified with column chromatography using dichloromethane/methanol in a ratio of 40/5 as an eluent. 1.31 g yellow solid was obtained in 61% yield. ¹H NMR (500 MHz, Chloroform-d) δ 8.10 (s, 2H), 4.10-4.02 (m, 4H), 4.00 (s, 6H), 3.64 (s, 6H), 1.75-1.63 (m, 4H), 1.47-1.21 (m, 20H), 0.91-0.84 (m, 6H). ¹³C NMR (126 MHz, CDCl₃) δ 155.93, 153.11, 151.88, 148.87, 148.10, 132.60, 124.81, 110.04, 42.15, 34.79, 32.30, 30.26, 29.72, 28.62, 27.52, 23.12, 14.56.

8,8′-(naphtho[2,3-c][1,2,5]thiadiazole-4,9-diyl)bis(3,7-dimethyl-1-octyl-3,4,5,7-tetrahydro-1H-purine-2,6-dione) (NT2). Theo8 (1,932 mg, 7 mmol) and 4,7-dibromobenzo[c][1,2,5]thiadiazole (1,032 mg, 3 mmol), pivalic acid (80 mg, 0.8 mmol) and dried Cs₂CO₃ (3 g, 9 mmol) were added into a 50 mL round bottom flask. 25 mL toluene was then adding to the flask, followed by degassing with N₂ flow for 10 min. Tris(2-methoxyphenyl)phosphine (122 mg, 0.194 mmol) and bis(dibenzylideneacetone)palladium(0) (80 mg, 0.088 mmol) were added to the solution under N₂ flow, and the solution turned purple. The flask was then sealed with a rubber stopper and heated to 100° C. After reacting for 1 day, the system was cooled and filtered. The organic phase was then concentrated under reduced pressure. The crude product was further purified with column chromatography using dichloromethane/methanol in a ratio of 40/5 as an eluent. 1.20 g red solid was obtained in 52% yield. ¹H NMR (500 MHz, Chloroform-d) δ 7.97 (ddd, 2H), 7.72-7.48 (m, 2H), 4.19-3.98 (m, 4H), 3.82 (s, 6H), 3.67 (s, 6H), 1.91-1.63 (m, 4H), 1.50-1.21 (m, 20H), 0.94-0.77 (m, 6H). ¹³C NMR (126 MHz, CDCl₃) δ 155.88, 151.91, 151.46, 148.96, 146.98, 134.70, 129.75, 126.76, 119.65, 109.58, 42.19, 33.95, 32.31, 30.37, 29.73, 28.64, 27.55, 23.12, 14.58.

### Stability of the Luminescent Molecules

As discussed above, while efficiencies of organic semiconductors have continued to increase, the stability of organic semiconductors is yet to be improved. The instability of organic semiconductors is strongly associated with the reactivity towards ground-state oxygen (³O₂) in the atmosphere. Organic semiconductors are believed to degrade via two major degradation pathways. In a first degradation pathway (“Degradation Pathway 1”), when an organic semiconductor molecule is excited by light, an electron will be promoted to the lowest unoccupied molecular orbital (LUMO) from the highest occupied molecule orbital (HOMO). When the LUMO is of higher energy in regard to the electron affinity (EA) of ³O₂ (−3.75 eV), it is energetically favored for the electron on the LUMO to transfer onto the conduction band of ³O₂. See, e.g., Journal of Materials Chemistry A, 2019, 7(41), 23361-23377, incorporated herein by reference in its entirety. This is followed by the formation of a radical cation of the π-conjugated moiety and a superoxide radical (O₂⁻), the latter of which initiates the degradation of the π-conjugated structure. Notably, singlet oxygen (¹O₂) has little effect on the degradation of the organic semiconductors. Therefore, without wishing to be bound by theory, it is believed that lowering the energy levels of organic semiconductors is crucial and effective in enhancing their stability. In a second degradation pathway (“Degradation Pathway 2”), damaging radicals can be generated with the presence of side chains, due to the increased reactivity between oxygen and methylene groups directly bonded to the π-conjugated structures or hetero atoms.

Considering both pathways of organic semiconductor degradation, without wishing to be bound by theory, it is believed lowering the LUMOs of luminescent molecules with electron-withdrawing side chains could increase their solution processability without significant impacts on their stability. As shown in Example 1, N-alkylated theobromine was used to successfully suppress aggregation caused quenching (ACQ) while simultaneously enhancing solution processability of organic semiconductors. After modification by N-alkylating theobromine, the resulting luminescent molecules dyes demonstrated strong fluorescence both in solution and in solid state. It is believed that alkylated theobromine is rich with electron-withdrawing groups, which lowers the LUMOs of the resulting molecules. Therefore, it is believed Degradation Pathway 1 could be suppressed by introducing N-alkylated theobromine onto organic dyes, thereby improving stability while also yielding materials with improvements in fluorescence and increased solution-processability.

To this end, two theobromine dyes, BT2 and NT2, were synthesized. Four other structurally representative organic dyes were included in the experiment to provide a comprehensive understanding of the relationship between structure and photostability.

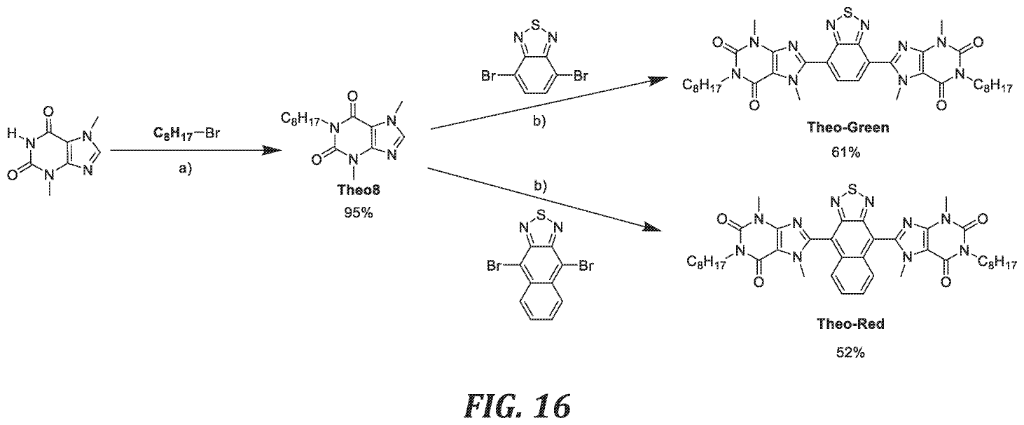

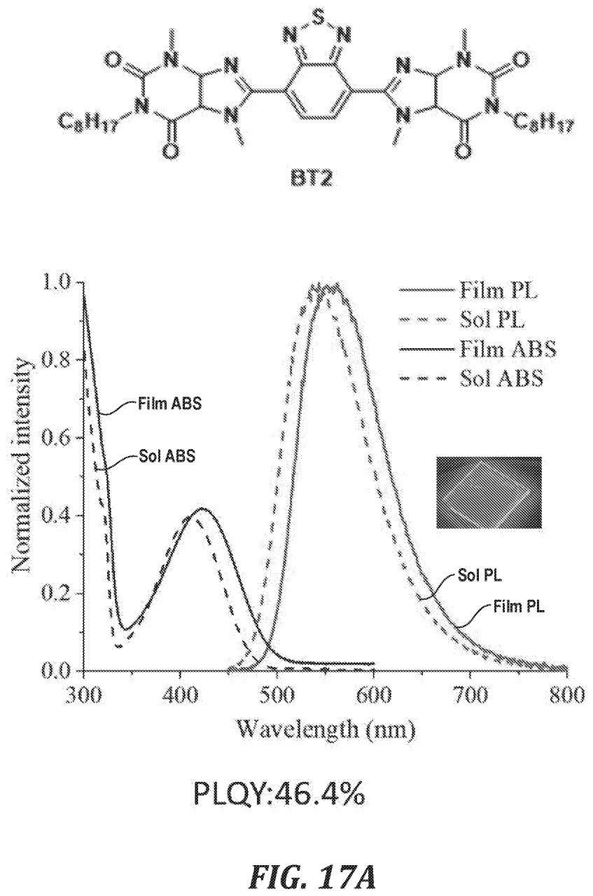

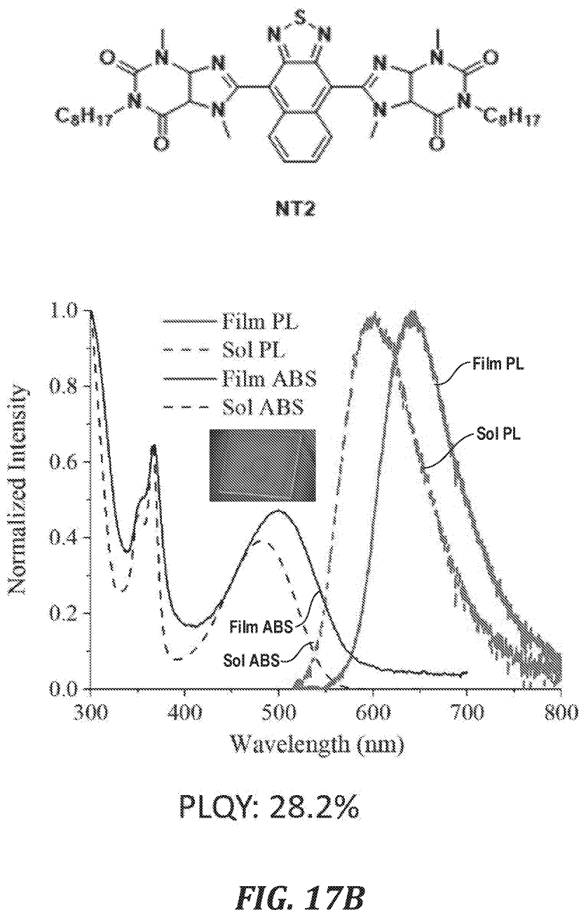

The theobromine dyes BT2 and NT2 were synthesized according to the reaction schemes in FIG. 16. To start, an octyl chain was covalently attached to theobromine to increase the solubility of the final luminescent molecules. Without wishing to be bound by theory, it is believed that the octyl group is chemically and optically inert, which is important to decrease or eliminate the likelihood of interference to the intrinsic stability and optoelectronic properties of the π-conjugated moieties. The reaction yield was quantitative. In addition to octyl group, various functional sidechains can be readily introduced onto theobromine in this step to tune the functionalities of resulting theobromine dyes, by switching to other halogenated reactants from octyl bromide. For example, 2-ethylhexyl bromide can provide higher solubility in organic solvents, brominated poly(ethylene glycol) can offer water solubility, 1-bromo-2,3-epoxypropane can allow for crosslinking and so on. Direct arylation, a green and atom-efficient cross-coupling methodology, was subsequentially performed to cross-couple the octyl theobromine with luminophores of interest to obtain final luminescent molecules with desired properties. Benzo[c][1,2,5]thiadiazole (BT) (see, e.g., Angewandte Chemie International Edition 2014, 53 (8), 2119-2123) and naphtho[2,3-c][1,2,5]thiadiazole (NT) (see, e.g., ACS Applied Materials & Interfaces 2019, 11 (26), 23417-23427) were selected as the luminophores. The cross-coupling reaction between octyl theobromine and BT and NT, respectively, and provided the green-emitting BT2 and the red-emitting NT2 in 61% and 52% yields. FIGS. 17A and 17B show the absorption and photoluminescence spectra of BT2 and NT2. Little overlap was observed between their absorption and emission spectra, which was characteristic for theobromine dyes. The subminimal overlap between absorption and emission, namely subminimal self-absorption, was important in increasing the light-emitting efficiency of final devices, such as lasers and luminescence solar concentrators.

External quantum efficiency (EQE) was used to quantify the light-converting efficiency of the phosphor films, which could be directly measured by an integrating sphere. The EQEs of spin-coated films of BT2 and NT2, were 46.4% and 28.2%. These were substantially higher than the precursors without alkylated theobromine, which were nearly 0% as thin films. External factors could further increase the EQEs and thus the overall efficiency of the final hybrid-LED.

Without wishing to be bound by theory, it is believed that the polarity of surrounding environments of organic dyes have strong influences on their EQEs—generally EQEs decrease with increasing polarity. Therefore, EQEs of BT2 and NT2 in solvents of different polarity were measured and summarized in Table 3. The EQEs of both BT2 and NT2 decreased as the polarity of the solvents increased, which was consistent with the general trend. This trend could be explained by the stabilization by high polarity environments of stabilized intramolecular charge transfer states of the excited molecules, where non-radiative decay is dominant. Taking this into account, the non-polar industrial polymer poly(styrene-butadiene-styrene) (SBS) was blended with the BT2 and NT2 to reduce the polarity of the light converting films. The good solution processability of the theobromine dyes and SBS significantly simplified the modification of the new film formulas. Dyes and SBS were dissolved into toluene in varied ratios and dropcasted onto glass substrates, with little change in the processing procedures used for thin films of neat organic dyes. Table 4 shows the EQE dependency of the dye-SBS phosphor film on the dye:SBS ratio, and slight decrease in EQEs were observed with increasing dye content. This was ascribed to the imide-rich polar structure of the theobromine dyes. The polarity of the dye-SBS composite increased as the dye content of the film increased. The EQEs of the theobromine dyes were sensitive to the polarity of the environments; which was attributed to a decrease in EQEs in higher dye concentration due to an increase of film polarity.

TABLE 3
The EQEs of BT2 and NT2 solution at
the concentration of 10⁻⁵M.

| | Relative polarity | BT2 | NT2 |
|---|---|---|---|
| Solvent | to water | [%] | [%] |
| Hexane | 0.009 | 96.7 | 91.3 |
| Toluene | 0.099 | 90.9 | 77.4 |
| Ether | 0.117 | 92.0 | 63.2 |
| Anisole | 0.198 | 80.5 | 43 |
| Chloroform | 0.259 | 77.5 | 45.1 |
| Dichloromethane | 0.309 | 71.8 | 62.6 |
| Acetonitrile | 0.460 | 2.4 | 0 |

TABLE 4
The EQEs of BT2 and NT2 SBS complex
at various blending ratio.

| Film composition | BT2 | NT2 |
|---|---|---|
| Dye:SBS weight ratio | [%] | [%] |
| 0.1:100 | 95 | 90 |
| 1:100 | 94 | 87 |
| 10:100 | 71 | 61 |
| 20:100 | 49 | 56 |

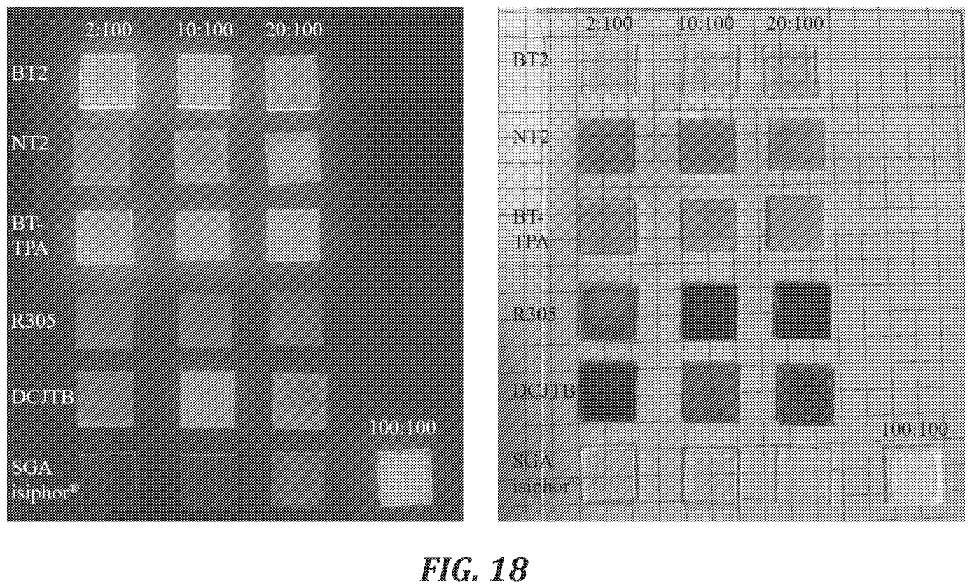

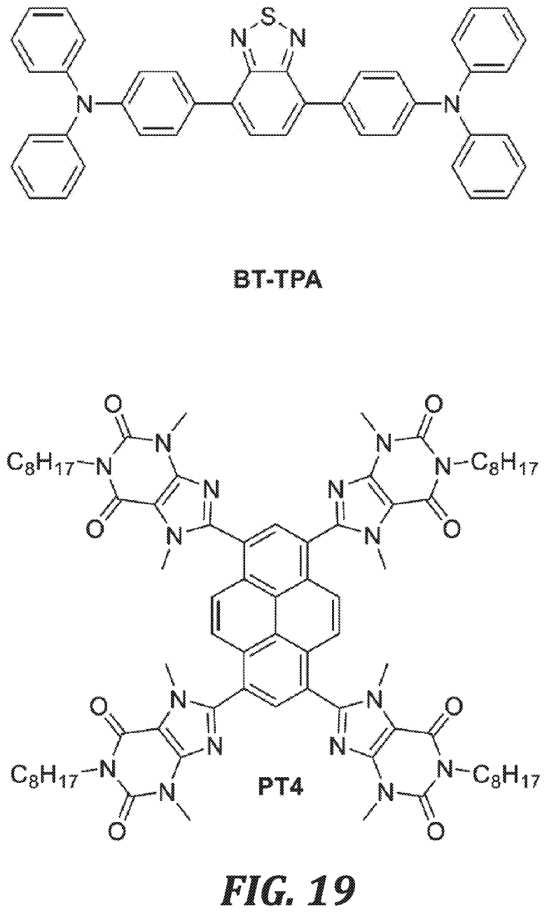

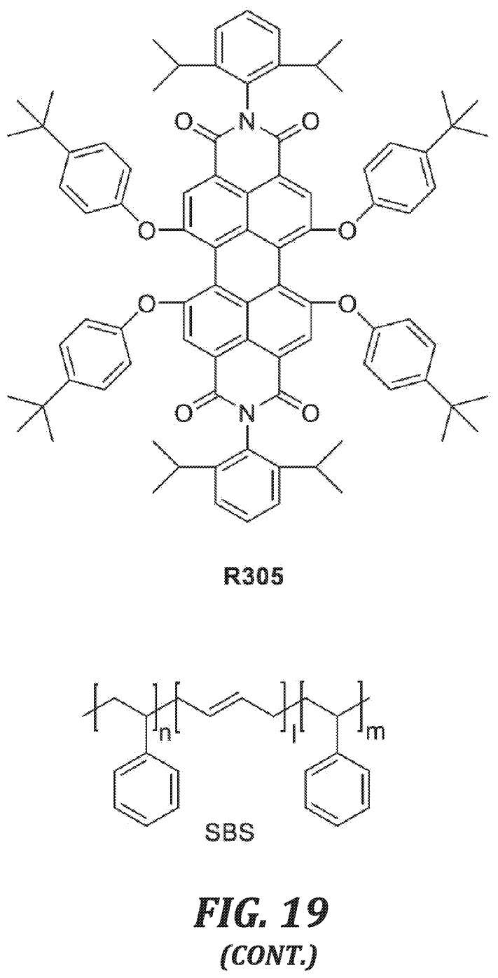

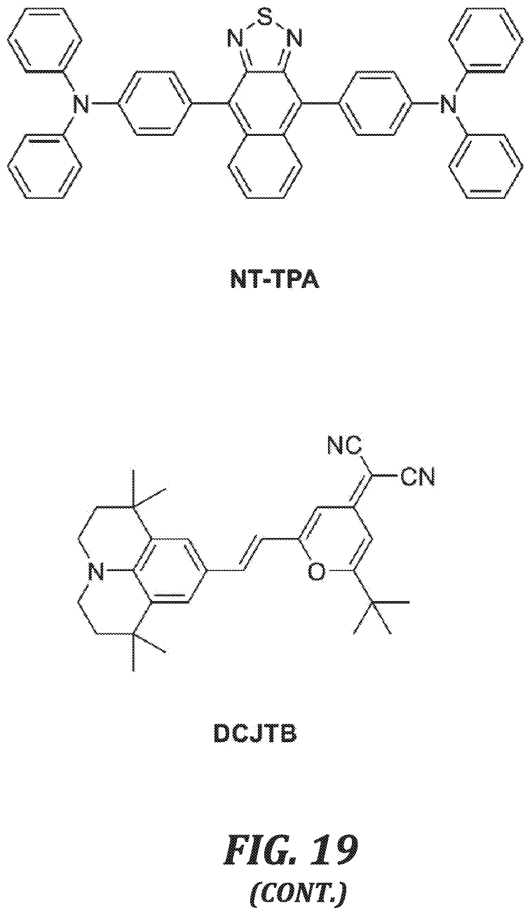

Even though the blending ratio of BT2 and NT2 was maintained at around 1% to maintain high EQEs around 90%, the low blending ratio was sufficient for hybrid-LED applications. FIG. 18 compares the efficacy of the theobromine dyes, other organic dyes (see, e.g., structures in FIG. 19) and inorganic phosphors in light-converting applications. The organic dye SBS samples were sufficient in converting light when the dye:SBS ratio was merely 1:100. However, the inorganic phosphor SGA Isiphor® from Sigma Aldrich required ˜100 times more materials to achieve comparable light intensity. This highlights the advantage of the theobromine dyes in light-converting applications regarding material consumption. Among organic dyes, the SBS blends of R305 and DCJTB had limited film transparency even at low doping ratio of 1:100. These two dyes had very planar π conjugated structures, and their intermolecular π-π interactions were facilitated the formation of large aggregates that scattered lights. SGA Isiphor®-SBS composite had low transparency as well. For both organic dyes and inorganic phosphors, low film transparency was disadvantageous in hybrid-LEDs because it allowed for significant scattering/reflection loss, which was responsible for reduced EQEs of the phosphor film and overall light output of hybrid-LEDs. Notably, because the size of traditional inorganic phosphor was usually of several microns, their scattering/reflection loss was inevitable and accountable for loss around 20% in light output. This was consistent with the fact that the state-of-art micron-size inorganic phosphors have EQEs around 70%, which was significantly lower than the theobromine dyes SBS composites. BT2, NT2 and BT-TPA had no scattering/reflecting loss under low blending ratios. However, only the BT2 maintained transparency at high dye:SBS ratio of 20:100; NT2 and BT-TPA became murky around 10:100. The role of side chains in solution processing organic dyes was underlined by these results. BT2 and BT-TPA had the same center aromatic unit (BT) and thus the difference in solubility/processability stemmed from their modifiers—alkylated theobromine was superior to non-alkylated TPA moieties. Compared with BT2, there was a decrease in solubility/processability in the NT2 sample. This was because the larger center aromatic unit (NT) enhanced π-π interactions that facilitated the formation of large aggregates, which could be circumvented by changing its octyl sidechain to a longer or branched side chain, thereby increasing its solubility in the SBS matrix and recovering the transparency of the dye-SBS composite.

The photostability of these organic dyes were compared and the relationship between structure and stability was investigated. 1) BT-TPA and BT2 both contained a BT core, however the triphenylamine (TPA) moieties in BT-TPA were electron-donating with no side chain. This was in sharp contrast to the electron-withdrawing alkylated theobromine, allowing comparisons of the two degradation pathways mentioned above. The TPA counterpart for NT2, NT-TPA, absorbed little light around 450 nm, which was the excitation wavelength used in the photostability experiment, and was therefore excluded in the study. 3) PT4 was chosen to better illustrate the impact of the LUMO's position on stability. Compared to BT2 and NT2, PT4 possessed a shallower LUMO as pyrene was electron-neutral while BT and NT were both electron-withdrawing. 4) R305 (Journal of Polymer Science Part A: Polymer Chemistry 2019, 57 (3), 201-215) and DCJTB (Scientific Reports 2015, 5 (1), 10697) were comprehensively studied organic dyes that were commercially available. While R305 had deep LUMO levels, the LUMO of DCJTB was significantly shallower due to the presence of the aryl amine moiety and therefore they had distinctly different photostability parameters. Notably, R305 was one of the most stable fluorescent dyes so far reported, with its EQE unchanged over 6-year outdoor exposure when embedded in poly(methyl methacrylate) PMMA matrix. See, e.g., physica status solidi (a) 2014, 211 (5), 1150-1154.

The energy levels of the organic dyes were subsequentially determined with UV-Vis absorption spectroscopy and ultraviolet photoelectron spectroscopy (UPS) in tandem. The absolute positions of their HOMOs (E_{HOMO}) were measured by UPS, while their bandgaps (E_{g}) were calculated from their thin-film absorption onset. The positions of their LUMOs can be estimated with E_{LUMO}=E_{HOMO}+E_{g}. Cyclic voltammetry (CV) was not considered in this study because the energy levels of organic semiconductors shift when in contact with polar solvents, which were necessary to prevent the dissolution of their thin films during CV measurements.

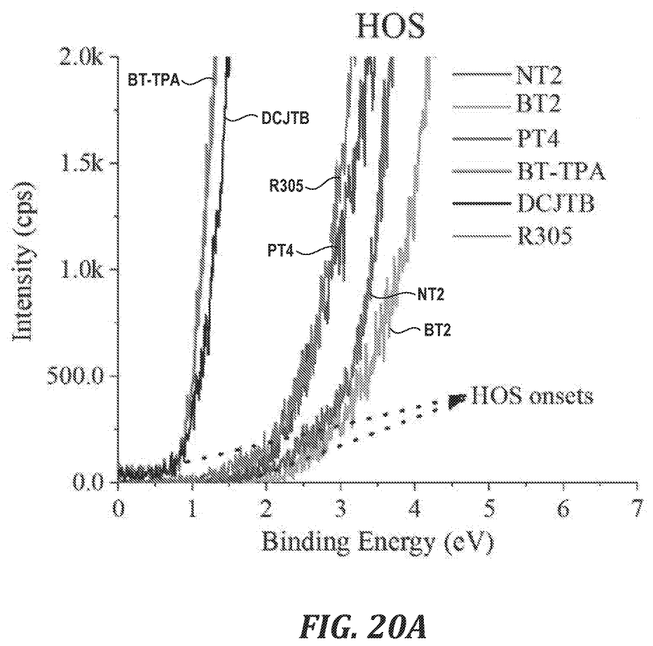

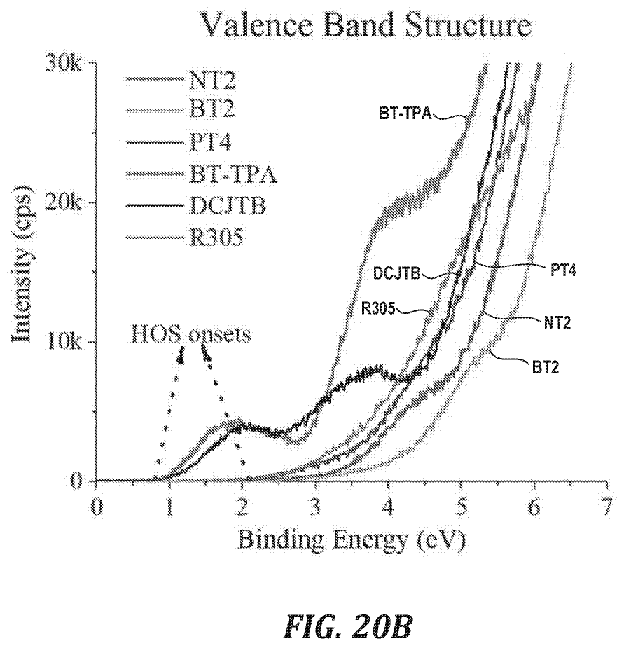

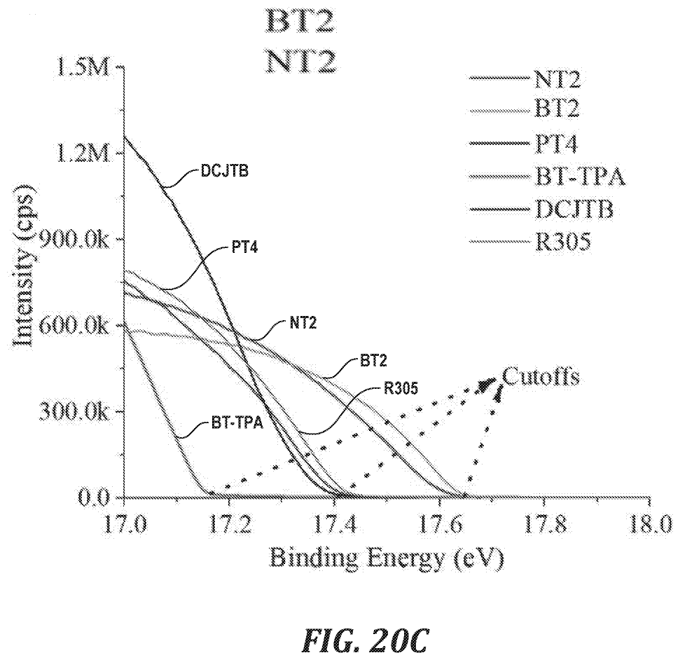

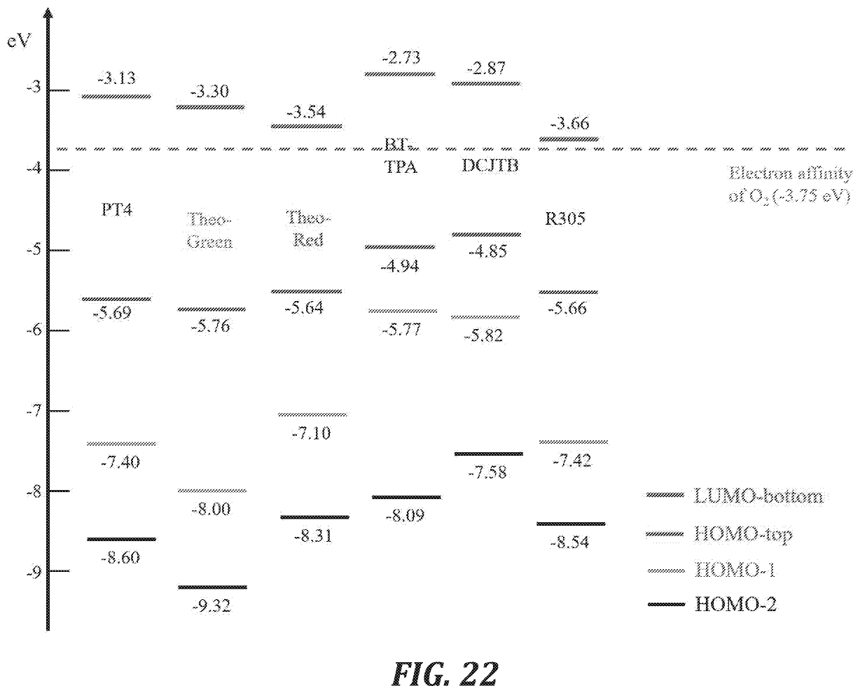

Shown in FIGS. 20A-20C are UPS He (I) spectra of the thin films made from BT2, NT2, BT-TPA, PT4, R305, and DCJTB. The energetic position of the top of their HOMO could be obtained with their highest occupied state (HOS) and cutoffs shown in FIGS. 20A-20C with this equation: E_{Top,HOMO}=21.2 eV−(E_{Cutoff}−E_{HOS}). Table 5 summarized these calculations. The top of HOMOs of BT-TPA and DCJTB were −4.94 eV and −4.85 eV regarding to vacuum level respectively, distinctly shallower than the other compounds. This could be ascribed to their extra valence bands centered at low binding energies as shown in FIG. 22, 1.75 eV and 2.00 eV respectively for BT-TPA and DCJTB. They corresponded to −5.77 eV (BT-TPA) and −5.82 eV (DCJTB), with respect to vacuum level. Considering that the lone pair electrons of trialkyl amines have oxidation potentials around −5.1 eV, the formation of these shallow band in BT-TPA and DCJTB could be attributed to the solitary tertiary amines in their conjugated systems. Although there were also tertiary amines present in the other organic dyes, their lone pair electrons were closely interacting with electron-withdrawing groups (carbonyl and imine), which led to the formation of 3-center 4-electron (3c-4e) π bond and the loss of their lone pair features. Therefore the other compounds do not have these extra band at lower binding energies.

TABLE 5
Energetic parameters of the six organic dyes.

| | | Band | Band | | HOMO- | HOMO- | HOMO- | Abs | | LUMO- |
|---|---|---|---|---|---|---|---|---|---|---|
| | HOS | 1 | 2 | Cutoff | top | 1 | 2 | λ_{cutoff} | Eg | bottom |
| | [eV]^{a)} | [eV]^{a)} | [eV]^{a)} | [eV]^{a)} | [eV]^{b)} | [eV]^{b)} | [eV]^{b)} | [nm]^{c)} | [eV]^{d)} | [eV]^{e)} |
| Theo- | 2.2 | 4.44 | 5.76 | 17.64 | −5.76 | −8.00 | −9.32 | 505 | 2.46 | −3.30 |
| Green | | | | | | | | | | |
| Theo- | 2.05 | 3.51 | 4.72 | 17.61 | −5.64 | −7.10 | −8.31 | 590 | 2.10 | −3.54 |
| Red | | | | | | | | | | |
| BT- | 0.92 | 1.75 | 4.07 | 17.18 | −4.94 | −5.77 | −8.09 | 561 | 2.21 | −2.73 |
| TPA | | | | | | | | | | |
| DCJTB | 1.03 | 2.00 | 3.76 | 17.38 | −4.85 | −5.82 | −7.58 | 627 | 1.98 | −2.87 |
| PT4 | 1.90 | 3.61 | 4.81 | 17.41 | −5.69 | −7.40 | −8.60 | 485 | 2.56 | −3.13 |
| R305 | 1.90 | 3.66 | 4.78 | 17.44 | −5.66 | −7.42 | −8.54 | 620 | 2.00 | −3.66 |

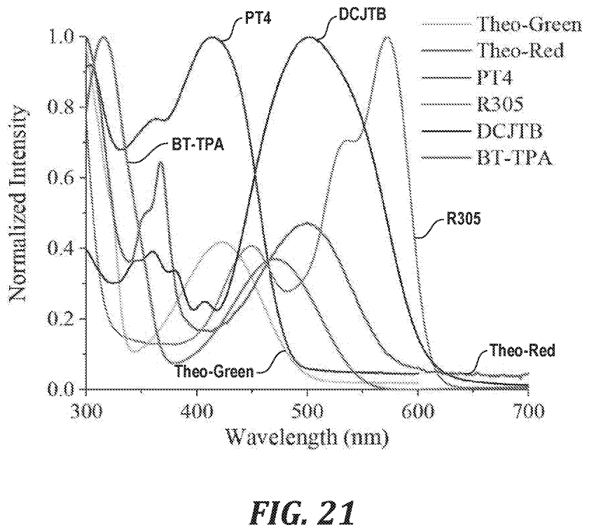

^{a)}Obtained from UPS results in FIGS. 20A-20C
^{b)}Calculated from the UPS results based on E_{Top,HOMO} = −21.2 eV + (E_{Cutoff} − E_{HOS});
^{c)}Obtained from UV-vis spectra in FIG. 21
${d )}^{} Calculated based on {E}_{g} = \frac{1240}{{λ}_{cutoff}} ;$ ^{e)}Calculated based on E_{Bottom,LUMO} = E_{Top,HOMO} + E_{g}

The position of the bottom of their LUMOs could be calculated with E_{Bottom,LUMO}=E_{Top,HOMO}+E_{g}), and the bandgaps of these six compounds were estimated by UV-vis absorption of their thin films according to

$${E}_{g} = \frac{1240}{{λ}_{cutoff}}$$

(see FIG. 21). Their LUMO positions were summarized in Table 5 and their partial band structures were visualized in FIG. 22. Two LUMO position trends could be observed in this data: 1) molecules had uplifted HOMOs, BT-TPA and DCJTB, and possessed shallow LUMO levels. 2) For molecules with similar HOMO positions—PT4, NT2 and R305—their LUMO positions deepened as their bandgaps decreased.

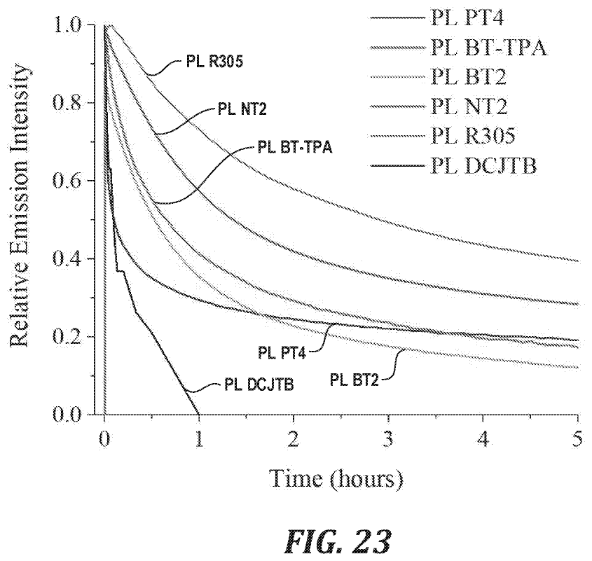

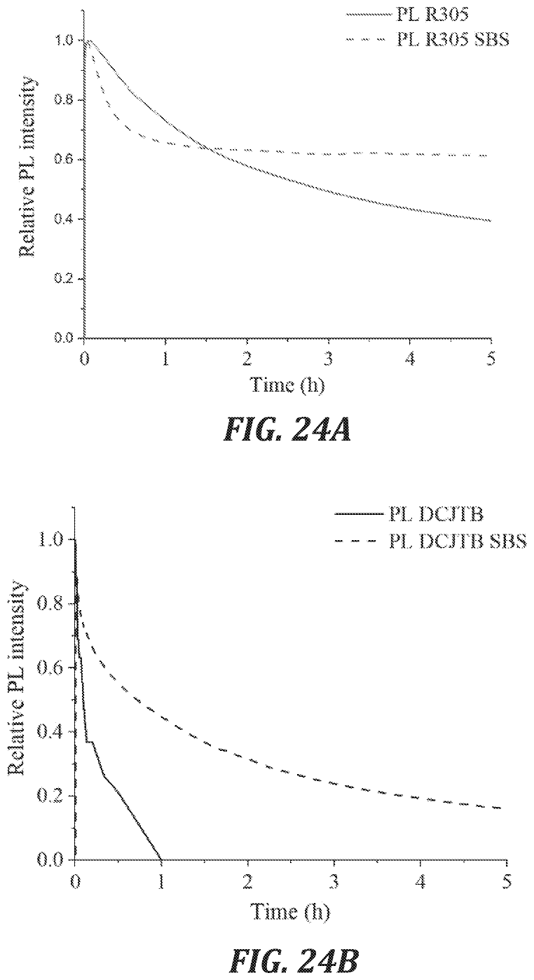

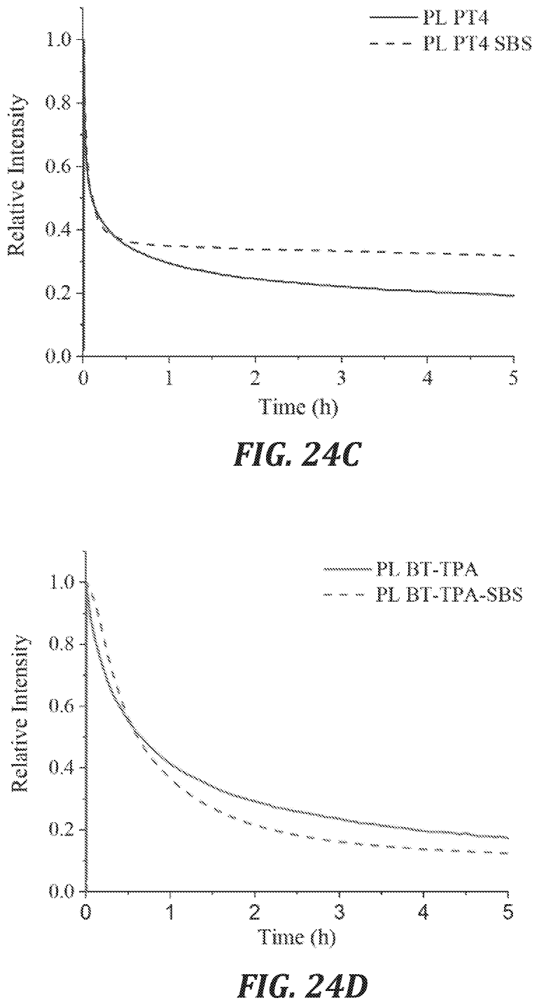

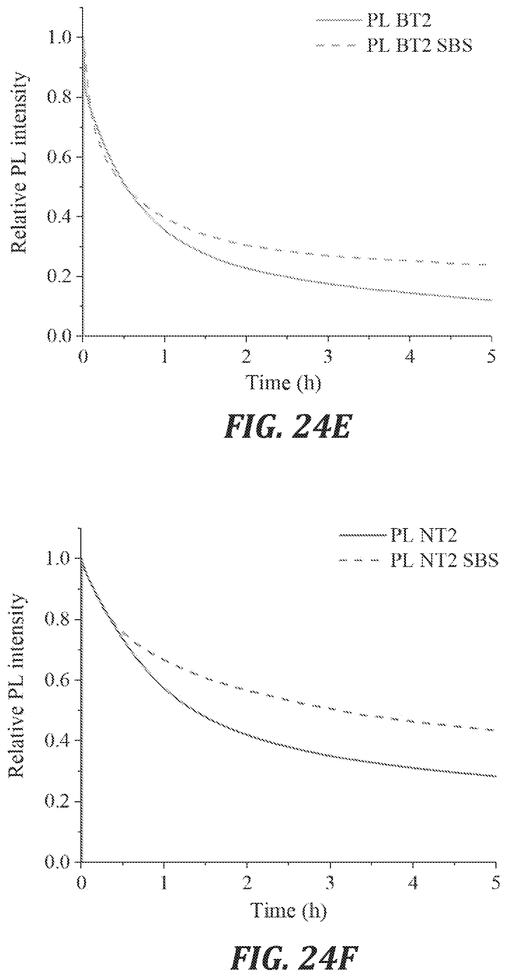

Subsequentially, photostability measurements were performed to investigate the LUMO-photostability relationship. The organic dyes were spin-coated into thin films under ambient condition. Subsequently, photostability measurements were carried out under ambient condition under the constant excitation of a 450 nm blue LED while the PL intensity of the organic dyes were monitored over time. A strong light intensity of ˜90 mW/cm² was applied in this experiment to accelerate the degradation experiments, to ˜100 mW/cm² of the grand total of AM1.5 sunlight of all wavelengths. FIG. 23 shows the evolution of PL intensity of the six dyes in a stability measurement. Two regions were observed in each PL decay curve for the six organic dyes, a fast decay region at approximately from 0 to 1 hour and a slow decay region at beyond 1 hour of radiation. The fast decay region was ascribed to the degradation of the molecules at the solid-air interface, while the slow decay region belonged to the degradation of the molecules that were further away from the solid air interface, where their degradation was limited by the diffusion of molecular oxygen. This was supported by our degradation experiment of dye-SBS composites, where the 0-1 hour decay speeds changed little compared to the neat films, while the speeds beyond 1 hour were mitigated (see FIGS. 24A-24F).

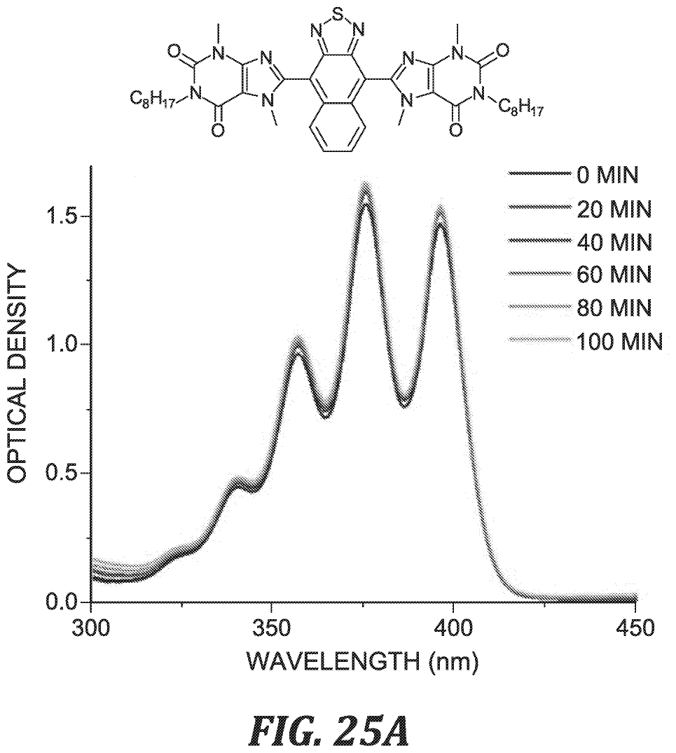

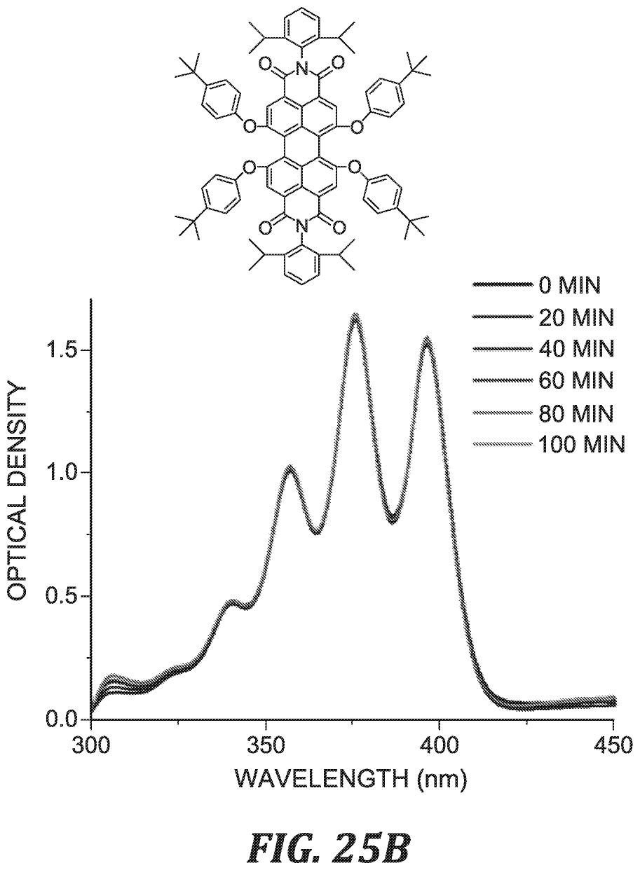

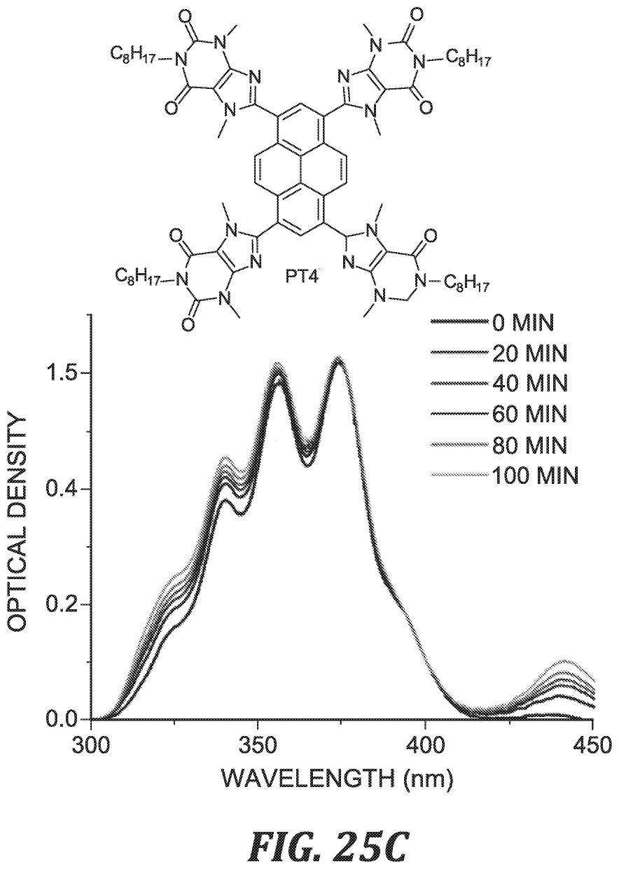

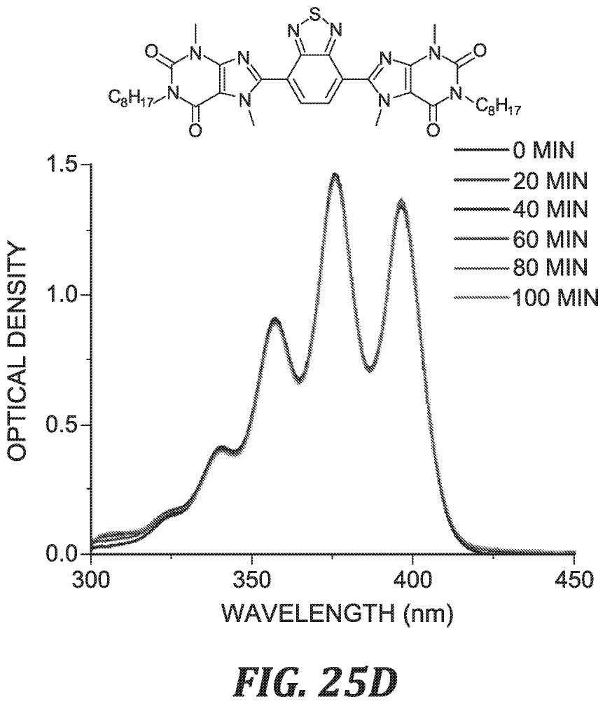

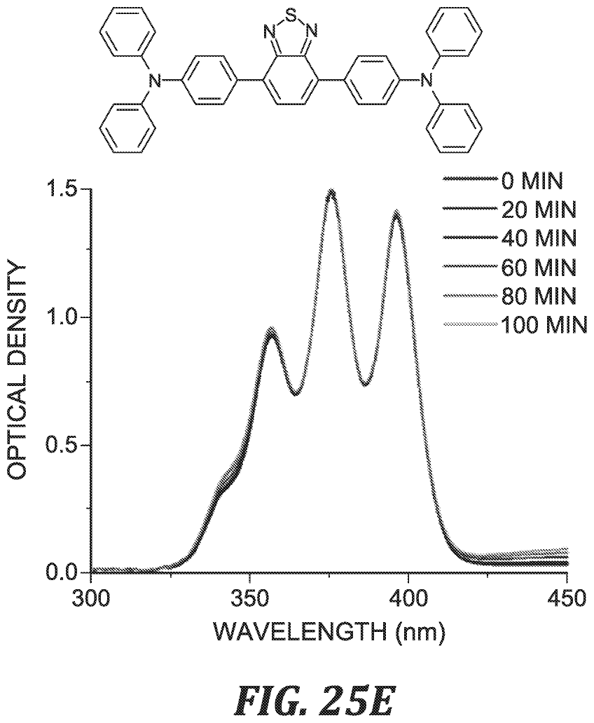

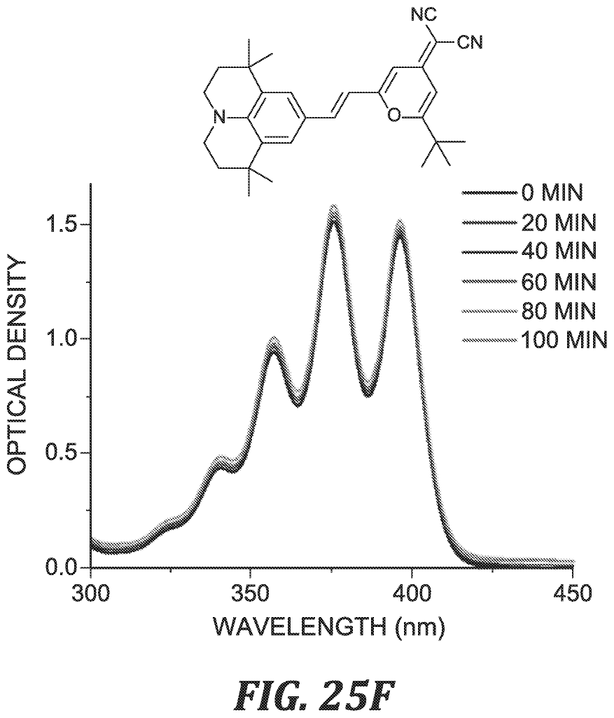

PT4, BT2, NT2 and R305 had similar structural features (amide groups and alkyl side chains) but different LUMO levels. R305 was observed to have the best stability among them, followed by NT2 and then BT2, and PT4 is the least stable, which was consistent with Degradation Pathway 1, discussed above—the deeper the LUMO, the better stability of the organic dyes. Among these structurally similar dyes, lower bandgaps resulted in enhanced stability—R305 had the lowest bandgap due to its largest center c moiety, which lowered its LUMO and enhanced photostability. However, solely lowering bandgaps cannot be applied in all situation because PL of the final luminescent molecule can change. On the other hand, DCJTB, BT-TPA and PT4 had shallow LUMOs around −3 eV and BT-TPA showed significantly strengthened stability with respect to DCJTB and PT4. This agreed with Degradation Pathway 2, where the presence of alkyl chain accelerated the photodegradation of organic dyes. In contrast, the stability of BT2 was comparable to BT-TPA, even with the six additional alkyl chains per molecules which should significantly facilitate Degradation Pathway 2. This indicated that Degradation Pathway 1 in BT2 was successfully suppressed with the modification of the electron-withdrawing theobromine to lower the LUMOs, and alkylated theobromine was a successful strategy to increase the solubility of organic dyes without undermining their photostability. Notably, the dye degradation mechanism from singlet oxygen formation was excluded because the formation of singlet oxygen was not observed from these six organic dyes under 450 nm radiation, as shown in FIGS. 25A-25F. Thus, in the present Example, a method to synthesize highly fluorescent luminescent molecules with enhanced photostability and tunable solution processability by introducing alkylated theobromine onto organic luminophores was demonstrated to be effective. The enhanced stability of these theobromine-functionalized dyes originated from their relatively deeper LUMO levels, which suppressed the formation of superoxide radicals under irradiation, because of the electron-withdrawing nature of theobromine. The solution processability of the final products was readily modified by adjusting the side chains attached onto theobromine, which played important roles in the fabrication and performance of the final devices. The resulting solution-processed light converting films demonstrated high EQEs of around 90% and wide spectral emissions, showing huge potentials for use in devices such as hybrid-LEDs with enhanced energy efficiency.

While illustrative embodiments have been illustrated and described, it will be appreciated that various changes can be made therein without departing from the spirit and scope of the invention.

1. A luminescent molecule, comprising:
a luminophore; and
a moiety comprising a purine core covalently and directly bonded to the luminophore, the purine core optionally substituted with 1, 2, 3, or 4 substituents independently selected from C═O, amino, C₁₋₂₄ alkyl, C₂₋₂₄ alkenyl, C₂₋₂₄ alkynyl, poly(alkylene oxide), aryl, heteroaryl, a polysiloxane, and any combination thereof, wherein each substituent is optionally substituted with 1, 2, 3, or 4 substituents independently selected from C₁₋₂₄ alkyl, C₂₋₂₄ alkenyl, C₂₋₂₄ alkynyl, halo, SH, cyclic ether, OH, CN, N₃, NCO, C(O)H, COOH, C(O)OR^{a}, C(O)NR^{a}, N═NR^{a}, aryl, heteroaryl, SO₃⁻, C—PO(OR^{a})(OR^{b}), NH₂, NHR^{a}, NR^{a}R^{b}, and (NR^{a}R^{b}R^{c})⁺, wherein each of R^{a}, R^{b}, and R^{c} is independently H, alkyl, aryl, arylalkyl, or a heterocycle,
wherein the luminescent molecule has an increased photoluminescence quantum yield relative to an analogous luminophore dye without a covalently bonded moiety comprising the purine core.

2. The luminescent molecule of claim 1, wherein the luminophore is selected from benzene, naphthalene, anthracene, phenanthrene, tetracene, fluoranthene, pyrene, pentacene, perylene, fluorene, carbazole, dibenzo[b,d]thiophene, dibenzo[b,d]furan, 1,10-phenanthroline, dibenzo[b,d]thiophene5,5-dioxide, [1,2,5]oxadiazolo[3,4-c]pyridine, [1,2,3]triazolo[4,5-c]pyridine, [1,2,5]selenadiazolo[3,4-c]pyridine, [1,2,5]thiadiazolo[3,4-c]pyridine, benzo[c][1,2,5]thiadiazole, benzo[c][1,2,5]oxadiazole, benzo[c][1,2,5]selenadiazole, naphtho[2,3-c][1,2,5]thiadiazole, naphtho[2,3-c][1,2,5]oxadiazole, naphtho[2,3-c][1,2,5]selenadiazole, benzo[d][1,2,3]triazole, naphtho[2,3-d][1,2,3]triazole, benzo[1,2-c:4,5-c′]bis[1,2,5]thiadiazole, benzo[1,2-c:4,5-c′]bis[1,2,5]oxadiazole, benzo[1,2-c:4,5-c′]bis[1,2,5]selenadiazole, benzo[1,2-c:4,5-c′] bis[1,2,5]triadiazole, triphenylamine, 5,5-difluoro-5H-4l4,5l4-dipyrrolo[1,2-c:2′,1′-f][1,3,2]diazaborinine, [1,2,5]thiadiazolo[3,4-f][1,10]phenanthroline, phenanthro[9,10-c][1,2,5]thiadiazole, dithieno[3′,2′:3,4;2″,3″:5,6]benzo[1,2-c][1,2,5]thiadiazole, 6,7-dihydropyrrolo[3,2-g][1,2,5]thiadiazolo[3,4-e]indole, furo[3′,2′:6,7]benzofuro[4,5-c][1,2,5]thiadiazole, [1,2,5]oxadiazolo[3,4-f][1,10]phenanthroline, phenanthro[9,10-c][1,2,5]oxadiazole, dithieno[3′,2′:3,4;2″,3″:5,6]benzo[1,2-c][1,2,5]oxadiazole, 6,7-dihydropyrrolo[3,2-g][1,2,5]oxadiazolo[3,4-e]indole, furo[3′,2′:6,7]benzofuro[4,5-c][1,2,5]oxadiazole, [1,2,5]selenadiazolo[3,4-f][1,10]phenanthroline, phenanthro[9,10-c][1,2,5] selenadiazole, dithieno[3′,2′:3,4;2″,3″:5,6]benzo[1,2-c][1,2,5]selenadiazole, 6,7-dihydropyrrolo[3,2-g][1,2,5]selenadiazolo[3,4-e]indole, furo[3′,2′:6,7]benzofuro[4,5-c][1,2,5]selenadiazole, 5,5′-bis(benzo[c][1,2,5]thiadiazol-4-yl)-2,2′-bithiazole, 5,5′-bis(benzo[c][1,2,5]thiadiazol-4-yl)-2,2′-bithiophene, benzo[de]isoquinoline-1,3(2H)-dione, lbenzo[lmn][3,8]phenanthroline-1,3,6,8(2H,7H)-tetraone, 2-methyl-1H-benzo[10,5]anthra[2,1,9-def]isoquinoline-1,3(2H)-dione, 2,9-dimethylanthra[2,1,9-def:6,5,10-d′e′f]diisoquinoline-1,3,8,10(2H,9H)-tetraone, naphtho[7,8,1,2,3-nopqr]tetraphene, and any combination thereof.

3. The luminescent molecule of claim 1, wherein the moiety comprising the purine core is a natural product or a derivative thereof.

4. The luminescent molecule of claim 1, wherein the moiety comprising the purine core is selected from a purine moiety, a theobromine moiety, a caffeine moiety, a xanthine moiety, a guanine moiety, an isoguanine moiety, a paraxanthine moiety, an adenine moiety, a theophylline moiety, a hypoxanthine moiety, and any combination thereof.

5. The luminescent molecule of claim 1, selected from:

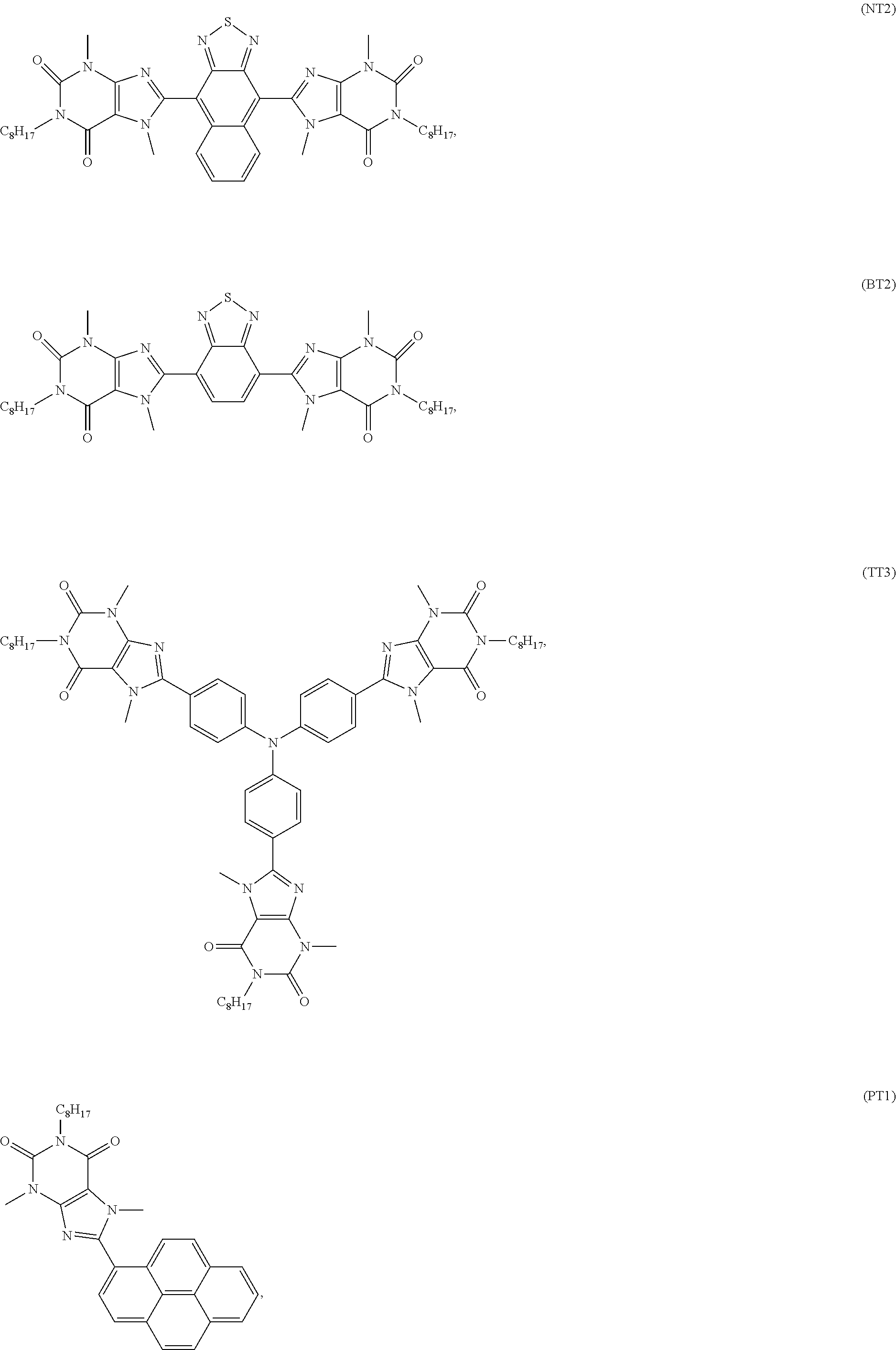

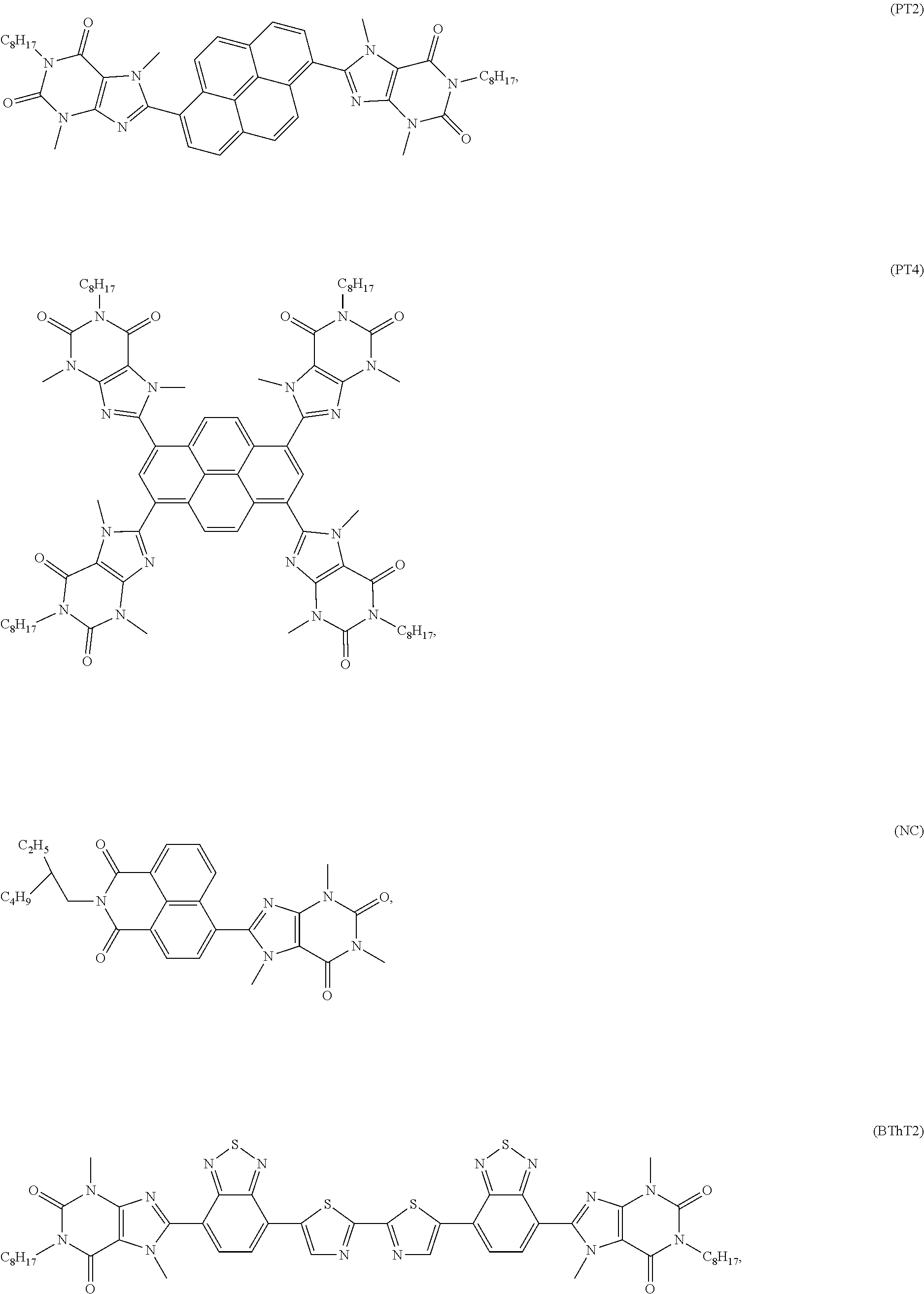

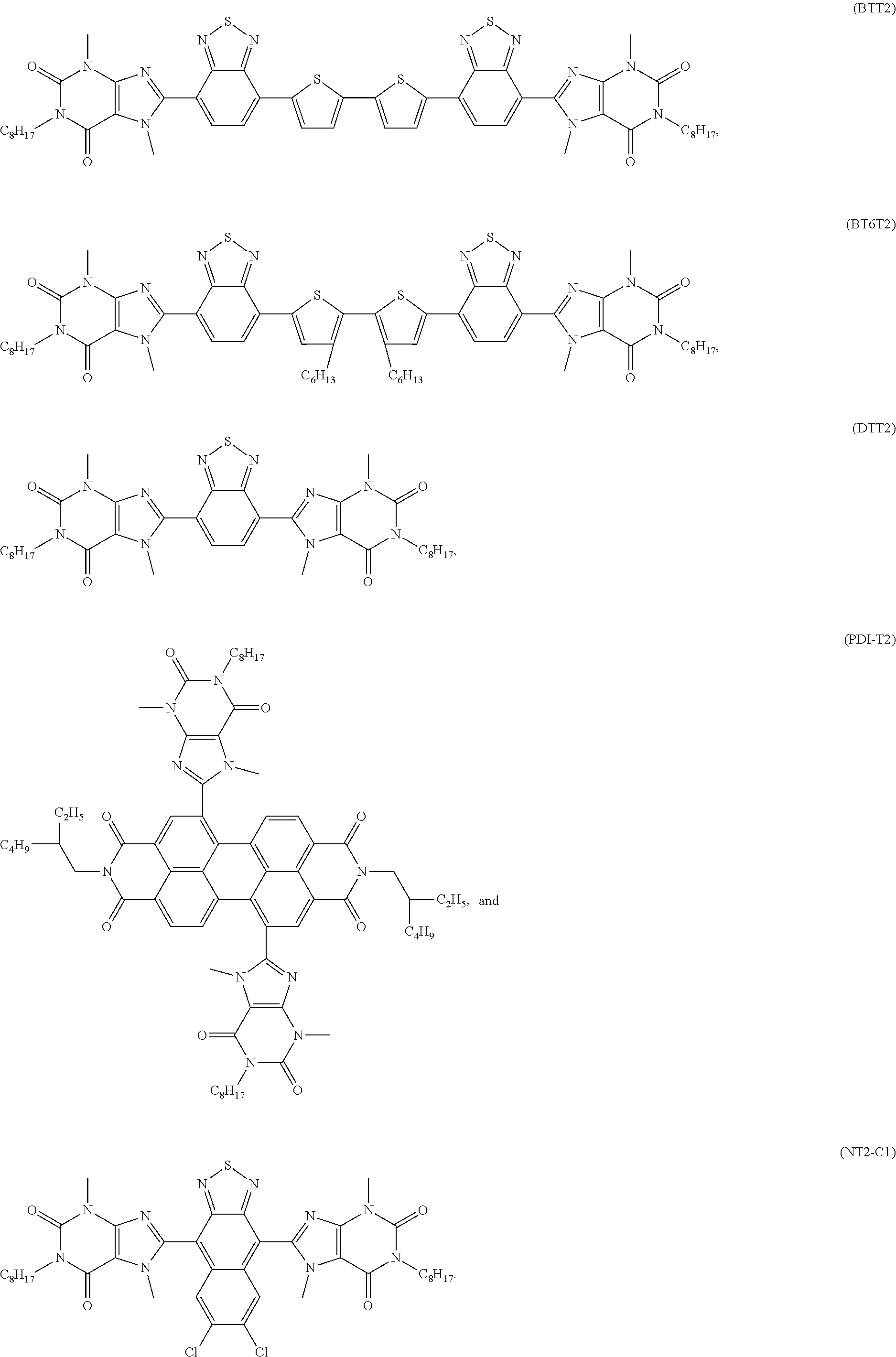

6. The luminescent molecule of claim 1 wherein the luminescent molecule has an increased photoluminescence quantum yield of at least 20% relative to an analogous luminophore without a covalently bonded moiety comprising a purine core in a solid state

7. The luminescent molecule of claim 1 wherein the luminescent molecule has an increased photoluminescence quantum yield of at least 20% relative to an analogous luminophore without a covalently bonded moiety comprising a purine core when the luminescent molecule is in a concentration of at least 10⁻³ M in solution.

8. The luminescent molecule of claim 1, wherein the luminescent molecule has an increased photoluminescence quantum yield of at least 20% relative to an analogous luminophore without a covalently bonded moiety comprising a purine core when the luminescent molecule is in a concentration of at least 10⁻³ M in a gel.

9. The luminescent molecule of claim 1, wherein the luminescent molecule has decreased aggregation quenching relative to an analogous luminophore without the covalently bonded moiety comprising a purine core.

10. A method of making a luminescent molecule of claim 1, comprising:
providing a reaction mixture comprising a luminophore comprising a leaving group covalently bound thereto, a compound comprising a purine core having an activatable C—H bond, and a palladium or copper catalyst;
activating the activatable C—H bond of the compound comprising the purine core with the palladium or copper catalyst to provide an activated compound comprising the purine core, and
reacting the luminophore comprising a leaving group covalently bound thereto and the activated compound comprising the purine core to provide a luminescent molecule of any one of claims 1 to 9.

11. The method of claim 10, wherein the luminophore comprising a leaving group covalently bound thereto comprises 1, 2, 3, or 4 leaving groups.

12. The method of claim 10, wherein the luminophore comprising a leaving group covalently bound thereto comprises 1 or 2 leaving groups.

13. The method of any one claim 10, wherein the leaving group covalently bound to the luminophore comprises a reactive halo substituent selected from chloro, bromo, and iodo.

14. The method of claim 10, wherein the palladium catalyst is present in an amount of 0.01M to 0.2M relative to the luminophore comprising a leaving group covalently bound thereto.

15. The method of claim 10, wherein the copper catalyst is selected from copper(I) acetate, copper(I) iodide, copper(I) bromide, copper(I) chloride, copper(I) bromide dimethyl sulfide complex, tetrakis(acetonitrile)copper(I) hexafluorophosphate, copper(I) oxide, tetrakis(acetonitrile)copper(I) tetrafluoroborate, copper(I) trifluoromethanesulfonate toluene complex, copper(I) trifluoromethanesulfonate benzene complex, copper(I) thiophene-2-carboxylate, copper(I) chloride-bis(lithium chloride) complex, iodo(triethyl phosphite)copper(I), copper (I) diphenylphosphinate, tetrakisacetonitrile copper(I) triflate, bromotris(triphenylphosphine)copper(I), (1,10-phenanthroline)(trifluoromethyl)copper(I), bis[(tetrabutylammonium iodide)copper(I) iodide], chloro(1,5-cyclooctadiene)copper(I) dimer, chloro[1,3-bis(2,6-diisopropylphenyl)imidazol-2-ylidene]copper(I), chloro[1,3-Bis(2,4,6-trimethylphenyl)imidazol-2-ylidene]copper(I), bis(1,3-bis(2,6-diisopropylphenyl)imidazol-2-ylidene)copper(I) tetrafluoroborate, iodo[4,5-bis(diphenylphosphino)-9,9-dimethylxanthene]copper(I), DABCO®-CuCl complex, and any combination thereof, in an amount of 0.01 to 0.5 M relative to the luminophore comprising a leaving group covalently bound thereto.

16-18. (canceled)

19. A device, comprising a luminescent molecule of claim 1.

20. The device of claim 19, selected from a light emitting diode, a laser, a solar concentrator, green house coverage, and a down-converting material.

21. A fluorescent biomolecule label, comprising a luminescent molecule of claim 1.

22. The fluorescent biomolecule label of claim 21, wherein the luminescent molecule is further functionalized with a reactive group suitable for bioconjugation.

23. The fluorescent biomolecule label of claim 21, wherein the reactive group suitable for bioconjugation is selected from NHS-ester, isocyanate, isothiocyanate, benzoyl fluoride, maleimide, iodoacetamide, 2-thiopyridine, 3-arylpropiolonitrile, carboxylate, phosphonate, sulfonate, and any combination thereof.
